SEQUENCE LISTING (1 sequence)

SEQ ID NO: 1 - DNA, 30 bp, Artificial Sequence
  Synthetic- Probe for visualization of rAAV genome
  misc_feature (30)..(30): Atto-565 fluorescent moiety
cttgaagaagtcgtgctgcttcatgtggtc

(19) United States
(12) Patent Application Publication
(10) Pub. No.: US 2022/0017978 A1
(43) Pub. Date: Jan. 20, 2022

(54) VIROFIND: A NOVEL PLATFORM FOR DETECTION AND DISCOVERY OF THE ENTIRE VIROGENOME IN CLINICAL SAMPLES

(71) Applicant: Northwestern University, Evanston, IL (US)

(72) Inventors: Igor J. Koralnik, Chicago, IL (US); Barbara A. Hanson, Chicago, IL (US); Xin Dang, Burr Ridge, IL (US)

(21) Appl. No.: 17/305,796

(22) Filed: Jul. 14, 2021

Related U.S. Application Data
(60) Provisional application No. 63/051,812, filed on Jul. 14, 2020.

(51) Int. Cl. C12Q 1/70

(52) U.S. Cl. CPC C12Q 1/701

(57) ABSTRACT

The invention relates to methods, systems, and components thereof for detecting and discovering viruses in a clinical sample. In particular, the invention relates to methods, systems, and components thereof for detecting and discovering a plurality of viruses in a clinical sample.

### CROSS REFERENCE TO RELATED APPLICATION

This application claims the benefit of priority under 35 U.S.C. § 119(e) of U.S. Provisional Application No. 63/051,812, filed Jul. 14, 2020, the contents of which is incorporated herein by reference in its entirety.

### STATEMENT REGARDING FEDERALLY SPONSORED RESEARCH OR DEVELOPMENT

This invention was made with government support under DA028493 and AG010161 awarded by the National Institutes of Health. The government has certain rights in the invention.

### SEQUENCE LISTING

A Sequence Listing accompanies this application and is submitted as an ASCII text file of the sequence listing named “702581_1981_ST25.txt” which is 688 bytes in size and was created on Jul. 14, 2021. The sequence listing is electronically submitted via EFS-Web with the application and is incorporated herein by reference in its entirety.

### FIELD OF THE INVENTION

The invention relates to methods, systems, and components thereof for detecting and discovering viruses in a clinical sample. In particular, the invention relates to methods, systems, and components thereof for detecting and discovering a plurality of viruses in a clinical sample.

### BACKGROUND

Viral infections, encompassing both acute and chronic viral infections, are a major public health issue. Often, viral infections are diagnosed without confirmatory knowledge of the specific pathogen causing the infection. In part, this is due to the current approaches to confirming viral infections, chiefly the use of PCR amplification of patient samples to detect specific viral nucleic acids that are indicative of the virus. However, such approaches require some a priori knowledge as to the suspected cause of the infection.

### SUMMARY

Disclosed herein are methods, systems, and components for detecting a plurality of RNA and DNA viruses in a human biological sample comprising RNA and DNA. In some embodiments, the method comprises: (i) performing reverse transcription of the RNA in the sample using a plurality of DNA primers to prepare double-stranded cDNA of the RNA; (ii) fragmenting the cDNA and DNA in the sample to prepare DNA fragments; (iii) treating the DNA fragments with enzymes that repair overhangs to obtain blunt-ended DNA fragments; (iv) treating the blunt-ended DNA fragments with an enzyme that adds a 3′ adenine overhang to the blunt-ended DNA fragments to obtain 3′-adenine extended DNA fragments; (v) ligating an adapter comprising an index sequence and a primer target sequence to the 3′-adenine extended DNA fragments to obtain adapter-ligated DNA fragments; (vi) amplifying the adapter-ligated DNA fragments with a plurality of DNA primer pairs that hybridize to the primer target sequence to obtain an amplified DNA sample; (vii) contacting the amplified DNA sample with a plurality of tagged RNA probes that hybridize to the amplified DNA sample to provide tagged RNA:DNA hybrid molecules; (viii) capturing the tagged RNA:DNA hybrid molecules using a molecule that binds to the tag of the tagged RNA:DNA hybrid molecules; (ix) amplifying the captured, tagged RNA:DNA hybrid molecules using a plurality of DNA primer pairs to obtain a further amplified DNA sample; (x) and analyzing the further amplified DNA sample based on the index sequence to detect the plurality of RNA and DNA viruses in the human biological sample.

In some embodiments of the disclosed methods, the DNA is fragmented by sonication. In some embodiments of the method, the fragmented DNA is on average between 50 and 300 base pairs in length.

In some embodiments of the disclosed methods, the enzymes of step (iii) have 5′-3′ polymerase activity and 3′-5′ exonuclease activity. In some embodiments of the disclosed methods, the enzyme of step (iv) is a polymerase. In some embodiments of the disclosed methods, the enzyme of step (iv) is Taq polymerase.

In some embodiments of the disclosed methods, the adapter that is ligated comprises an index sequence. In some embodiments of the disclosed methods, the index sequence is 5 to 15 nucleobases in length.

In some embodiments of the disclosed methods, the number of cycles of amplification of step (vi) is tuned based on the concentration of the adapter ligated fragments. In some embodiments of the disclosed methods, the number of cycles of amplification of step (vi) is tuned such that the adapter ligated fragments are amplified to an appropriate concentration.

In some embodiments of the disclosed methods, the amplified DNA sample is concentrated. In some embodiments of the disclosed methods, the amplified DNA sample is concentrated to at least about 215 ng/μl.

In some embodiments of the disclosed methods, the tagged RNA probes of step (vii) are designed to bind to a genomic segment of multi-partite viral genomes. In some embodiments of the disclosed methods, the tagged RNA probes of step (vii) are designed to bind to a genomic segment which encodes the viral capsid.

In some embodiments of the disclosed methods, the tagged RNA probes of step (vii) are designed to bind to the viral genome of a coronavirus. In some embodiments of the disclosed methods, the tagged RNA probes of step (vii) are designed to bind to the viral genome of SARS-CoV-2.

In some embodiments of the disclosed methods, the tagged RNA probes of step (vii) are designed to bind to the L1 gene sequence of a papilloma virus. In some embodiments of the disclosed methods, the tagged RNA probes of step (vii) are designed to bind to the L1 gene sequence for every known human papilloma virus.

In some embodiments of the disclosed methods, the tagged RNA probes of step (vii) are tagged with one member of a cognate pair of binding molecules. In some embodiments of the disclosed methods, the tagged RNA probes of step (vii) are tagged with biotin. In some embodiments of the disclosed methods, the tagged RNA probes of step (vii) are tagged with digoxigenin (DIG).

In some embodiments of the disclosed methods, the hybridization of step (vii) occurs at a temperature of about 55-75 degrees Celsius. In some embodiments of the disclosed methods, the hybridization of step (vii) occurs at a temperature of about 60-70 degrees Celsius.

In some embodiments of the disclosed methods, the hybridization of step (vii) is incubated for at least about 6 hours. In some embodiments of the disclosed methods, the hybridization of step (vii) is incubated for at least about 12 hours, 18 hours, or 24 hours.

In some embodiments of the disclosed methods, the RNA probes of step (viii) are tagged with biotin and streptavidin is utilized as a binding partner to bind to the biotin-tagged RNA:DNA hybrid molecules. In some embodiments of the disclosed methods, the RNA probes of step (viii) are tagged with biotin and streptavidin is utilized as a binding partner to capture the biotin-tagged RNA:DNA hybrid molecules

In some embodiments of the disclosed methods, the RNA probes of step (viii) are tagged with digoxigenin and anti-digoxigenin is utilized as a binding partner to bind to the digoxigenin-tagged RNA:DNA hybrid molecules. In some embodiments of the disclosed methods, the RNA probes of step (viii) are tagged with digoxigenin and anti-digoxigenin is utilized as a binding partner to capture the digoxigenin-tagged RNA:DNA hybrid molecules.

In some embodiments of the disclosed methods, the molecule that binds to the tag of the tagged RNA:DNA hybrid molecules is linked (e.g., covalently) to a solid substrate such as a bead. In some embodiments of the method, the beads are magnetic.

In some embodiments of the disclosed methods, step (x) comprises next-generation DNA sequencing. In some embodiments of the disclosed methods, the methods include a step of sequencing the further amplified DNA sample wherein DNA sequencing comprises paired-end sequencing.

### BRIEF DESCRIPTION OF THE FIGURES

FIG. 1. Non-limiting example ViroFind work-flow: Exemplified is an in-solution target-enrichment platform for virus detection and discovery in clinical samples. Sample DNA and cDNA are sonicated to a target fragment size of 150-200 bp, ligated with indexing adapters, and minimally amplified, prior to in-solution hybridization with biotinylated RNA probes. DNA:RNA hybrids are isolated through streptavidin-coated magnetic bead selection. Viral sequences are amplified post-capture prior to paired-end NextSeq sequencing analysis and characterization via the ViroFind bioinformatics analysis pipeline.

FIG. 2. Shows an example pipeline for bioinformatics analysis of samples using the Virofind 2.0 method.

FIG. 3A-C. AAV Genome, A) Wild-type AAV genome is just under 4.7 kb and contains two genes, rep and cap, which are flanked by inverted terminal repeat sequences (ITRs). rep and cap can be supplied in trans along with adenovirus helper genes during production and replaced with a transgene of interest. B) the 145 bp AAV ITR is a single-stranded palindromic T-shaped DNA molecule. Putative tau-binding site is shown. C) Top 10 tau-binding sites within the mouse genome are shown (113), with the top site and deviations in bold and the ITR binding site shown.

FIG. 4A-C. AAV is enriched in supramarginal gyrus of 10 AD subjects as compared to 10 controls. (A) Heatmap depicting all viral taxa identified by ViroFind analysis in this assay. Log 2 gradient scale indicates number of viral reads. (B) Frequency for each viral species for AD subjects and controls are shown. (C) Mean read count for each viral species for AD subjects and controls are shown. Mean AAV2 read count was 342, with average genome coverage of 90.3%, at mean sequencing depth of 14.6 (9.1-20.2).

FIG. 5A-D. Tbr2 detection in flash frozen paraffin embedded (FFPE) sections of human brain. A,B) Tbr2 expression in the OSVZ and ISVZ of fetal neocortex was detected by immunofluorescence (A) and IHC (B). C,D) TBR2 was detected in the DG, mainly the hilus, by immunohistochemistry (IHC) (and by immunofluorescence (IF), not shown). The boxed region in (C) is shown at higher magnification in (D). Some Tbr2+ nuclei appeared to form doublets, consistent with intermediate progenitor amplification. Scale bars: (A) 100 μm; (B) 200 μm; (C) 100 μm for (C), 300 μm for (D).

FIG. 6. Ablation of adult neurogenesis is dose dependent. Left: Experimental time course Middle: Representative images showing Prox1 and BrdU labeling 4 weeks post-injection. Right: Near complete ablation of BrdU+ cells is observed in the dentate gyrus (DG) injected with 1 μL 3 E12 GC/mL rAAV, with increased observed cell loss correlated with increasing viral dose administered.

FIG. 7A-G. Post-mitotic age of adult-born DGCs effects rAAV toxicity. (A) Experimental time course (B) Representative images showing Prox1 and BrdU labeling at different pre-injection intervals. (C) BrdU given for 3 days immediately (0 wk) preceding viral injection shows near complete elimination following rAAV injection; cells born 1 week before viral injection are reduced by ˜50%. Cells born >2 weeks after rAAV show no reduction. (D) TEM image of empty AAV capsids E) injected immediately after BrdU show no loss of BrdU+ cells when sacrificed 1 week later. F) BrdU+ cells show variable decline 12 hours after rAAV injection and significant decline at 18 hours relative to saline. G) Caspase-3+ apoptotic cells were increased in number relative to saline-injected controls at 12 hours.

FIG. 8A-G. NPC developmental stage determines susceptibility to rAAV-induced cell loss. (A) Experimental design. Following labeling with BrdU, mice are injected unilaterally with 1 μL 3 E12 gc/mL rAAV and sacrificed 2 days, 1 week, or 4 weeks later. (B) Representative images of Sox2 (upper panels), DCX (upper panels), and Tbr2 (lower panels) following rAAV injection. (C) Sox2+ population within the SGZ is reduced by ˜20% 2 days and 1 week following rAAV injection, but not at 4 weeks post-injection. (D) The majority of Tbr2+ cells are lost within 2 days of rAAV injection while (F) the late marker DCX shows progressive decline until complete loss at 4 weeks post-injection. (E, G) In contrast, Tbr2+ intermediate progenitors & DCX+ cells are preserved following injection of empty viral capsid.

FIG. 9A-C. rAAV toxicity in vitro. (A) rAAV at MOI of 1 E7 virus/cell arrests NPC proliferation by 24 h. MOI of 1 E6 results in slower proliferation relative to H₂O. (B) MOI 1 E7 and 1 E6 result in increased proportion of propidium iodide (PI) positive NPCs. (C) Representative images showing confluence (brightfield) and PI penetration (red) into NPCs 12 and 48 hours post-transduction for MOI of 107, 106, and for H₂O.

FIG. 10A-E. AAV ITR induces toxicity. (A) Experimental design for ITR electroporation. Mouse NPCs are electroporated with 5 E6 or 1 E6 copies of 145 bp ssDNA AAV2 ITR or scrambled ITR DNA per cell and plated for time lapse imaging or FACS. (B) 5 E6 ITR is causes cell loss within hours arrests growth by 40 h. 5 E6 scrambled ITR shows slight decrease in confluence relative to 1 E6 scrambled ITR, which is indistinguishable from H₂O (C) Electroporation of ITR results increased cell death at 5E6 copies/cell. (D) FACS demonstrates dose-dependent toxicity of 5 E6 ITR in replicating NPCs, where cells in S- and G2-phase are dying and are UVZombie+ at 12 hours. (E) NPCs in G1 represent the vast majority of cells (data not shown) and are not undergoing cell death.

FIG. 11. rAAV infection induces p-tau. AAV1-CAG-flex-eGFP injected into DG on the right results in significant increase in p-tau (AT8) 4 weeks post injection compared to contralateral side.

### DETAILED DESCRIPTION

Viruses are often suspected but rarely detected in patients presenting with inflammation of the brain (encephalitis), meninges (meningitis), or spinal cord (myelitis). In addition, viruses have been implicated in the pathogenesis of degenerative or inflammatory diseases of the nervous system including: Alzheimer's, Parkinson's, Amyotrophic Lateral Sclerosis, and Multiple Sclerosis.

The limiting factor of the current detection method by polymerase chain reaction (PCR) is the need to target viruses separately, i.e. one virus/one test, which is costly and inefficient. Metagenomic sequencing is limited due to the tremendous imbalance between the size of human cellular genomic DNA compared to that of viral genomes, precluding detection of low level viral infection.

To fulfill this unmet need, the inventors have developed a target-enhanced Next Gen sequencing-based platform and bioinformatics analysis pipeline. This method, in some embodiments, can detect up to 561 species of viruses that can infect humans, or cause zoonosis, in clinical samples. In addition, the method can, in some embodiments, detect viral variants/mutants and, potentially, novel viruses associated with human disease. Finally, the method, in some embodiments, can identify the site of integration of viruses in the human genome. Viral integration can lead to disruption of the host DNA sequence. This is caused by the insertion of exogenous viral DNA and can lead to alterations in coding and regulatory sequences that may potentially cause human disease.

Applications of the disclosed technology include, but are not limited to: (i) detection of 561 species of viruses known to infect humans or cause zoonosis, in clinical samples; (ii) early detection and characterization of virus outbreak for prevention of epidemics/pandemics; (iii) characterization of viral variants/mutants and their association with human diseases; (iv) discovery of novel viruses and their association with human diseases; (v) characterization of site of integrations of viruses in the human genome, which could disrupt normal metabolic pathways and cause diseases; (vi) exploratory tool to identify viruses as causal agents, co-factors or biomarkers of degenerative or inflammatory diseases of the nervous system like Alzheimer's Parkinson' Amyotrophic Lateral Sclerosis, Multiple Sclerosis etc.; the disclosed technology could also be used as a research tool to detect viral infection in conditions including autism, schizophrenia, rheumatoid arthritis, Crohn's disease, as well as various types of cancers; and (vii) characterization of viruses carried in mosquitoes to predict mosquito-borne viral diseases in human populations living in certain geographic areas.

Advantages of the disclosed technology include, but are not limited to: (i) unbiased detection of all viruses known to infect humans in a single clinical sample; (ii) enrichment of viral sequences present in clinical samples using custom-made biotinylated viral RNA probes; (iii) analysis of the entire viral genome rather than short fragments obtained by PCR; (iv) characterization of viral variants/mutants and potentially, novel viruses; (v) applicable to wide variety of clinical samples: Plasma, serum, white blood cells, sputum, spinal fluid, urine, and any organ tissue, either fresh or frozen; and (vi) amplification of viral signal >100 times compared to metagenomic sequencing.

In some embodiments, ViroFind may be, for example, an in-solution target-enrichment platform for virus detection and discovery in clinical samples. In one embodiment, ViroFind comprises 131,706 viral probes (8.415 Mbp) with mean genome coverage of 89.39% of 561 selected DNA and RNA viruses. These comprise all viruses known to infect humans or cause zoonosis. In some embodiments, sample DNA and cDNA are sonicated to a target fragment size of 150-200 bp, ligated with indexing adapters, and minimally amplified, prior to in-solution hybridization with biotinylated ViroFind RNA probes.

In some cases, fragmentation is performed after reverse transcription. Suitable methods for fragmenting DNA include physical methods (e.g., using sonication, acoustics, nebulization, centrifugal force, needles, or hydrodynamics), enzymatic methods (e.g., using NEBNext dsDNA Fragmentase from New England BioLabs), and tagmentation (e.g., using the Nextera™ system from Illumina).

A size selection step may subsequently be performed to enrich the library for fragments of an optimal length or range of lengths. Traditionally, size selection was accomplished by separating differentially sized fragments using agarose gel electrophoresis, cutting out the fragments of the desired sizes, and performing a gel extraction (e.g., using a MinElute Gel Extraction Kit™ from Qiagen). However, size selection is now commonly accomplished using magnetic bead-based systems (e.g., AMPure XP™ from Beckman Coulter, ProNex® Size-Selective Purification System from Promega).

In some embodiments, DNA:RNA hybrids are isolated through streptavidin-coated magnetic bead selection. In some embodiments, Viral sequences are post capture amplified prior to paired-end NextSeq sequencing analysis and characterization. By way of example but not by way of limitation, sequences are then analyzed through three bioinformatics pipelines: 1) Detection pipeline for identification of known viruses. 2) Discovery pipeline for identification of viral variants/mutants or potentially, novel viruses. 3) Integration pipeline for identification of site of integration of viruses in the human genome.

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “metagenomics” is the study of the genomes of multiple organisms that make up a community. Therefore by extension, “metagenomic sequencing” refers to the sequencing of the genomes that comprise a community of organisms. By necessity, metagenomic sequencing requires assembly of the nucleic acid sequences acquired by sequencing into discrete genomes to determine the identity of constituent members.

As used herein, “index” or “index sequence” refers to a nucleotide sequence that has a known identity, that is unique compared to other known sequences, and that corresponds to a particular sample. In other words, an index sequence should be designed such that the identity of the sample from which the nucleotides to which it is attached can be determined after sequencing occurs. Index sequences facilitate the multiplexing of samples into a single sequencing reaction, conserving time and reagents.

As used herein, “primer” refers to a single stranded DNA or RNA oligonucleotide that is designed, in some cases, to bind specifically to a single complementary DNA or RNA sequence and be used as a first template by DNA polymerase or reverse transcriptase enzymes to extend the nucleic acid sequence. For example, primers may be used to initiate polymerization of a single nucleotide during polymerase chain reaction (PCR). In some embodiments, primers may comprise an index sequence.

As used herein, “probe” refers to oligonucleotides that are tagged with a detectable moiety, that contain a region complementary to nucleic acid sequences of interest sufficient to bind (hybridize to) the nucleic acid sequences of interest and provide a means for their enrichment through the use of a capture reagent that specifically binds to the detectable moiety linked to the probe. In one example, the detectable moiety is biotin and the capture moiety is streptavidin (biotin:streptavidin). Other examples of similar capture reagent pairs include but are not limited to: antigen:antibody, digoxigenin:anti-digoxigenin antibody, various chemical pairs that comprise the class of affinity reagents known as covalent click chemistry.

As used herein, “ligation” refers to the process of allowing substantially complementary nucleotide sequences to associate and bind, often at a defined temperature for a defined time period. In some embodiments, for example, ligation may take place at for example, 60, 61, 62, 63, 64, 65, 66, 67, 68, 69, or 70 degrees C. In some embodiments, for example, ligation may take place over 18 hours. In some embodiment, for example, ligation may take place overnight. In some embodiments, for example, ligation may take place over 10, 11, 12, 13, 14, 15, 16, 17, 18, 19, 20, 21, 22, 23, 24, 25, or 26 hours. In some embodiments, for example, ligation may take place at 65 degrees C. for 18 hours.

As used herein, “capture”, also referred to as “affinity capture”, refers to the enrichment of a particular molecule of interest that is linked to a detectable moiety by reversible binding to a capture moiety that binds specifically to the detectable moiety with high affinity. Examples of detectable moiety:capture moiety include but are not limited to biotin:streptavidin, antigen:antibody, digoxigenin: anti-digoxigenin antibody, various chemical pairs that comprise the class of affinity reagents known as covalent click chemistry.

As used herein, “multi-partite” refers to viruses that are “segmented” with each segment of the viral genome present in a different viral particle. Therefore, “multi-partite viral genome” refers to the genome encoding a multi-partite virus.

As used herein, “capsid” or “viral capsid” refers to the protein shell that encloses the genetic material encoding a virus. Capsids consist of repeating structural units that arrange to form the final capsid. The capsid has at least three functions: 1) it protects the nucleic acid from digestion by enzymes, 2) contains special sites on its surface that allow the virion to attach to a host cell, and 3) provides proteins that enable the virion to penetrate the host cell membrane and, in some cases, to inject the infectious nucleic acid into the cell's cytoplasm.

As used herein, “L1 gene sequence” refers to the nucleic acid, DNA, cDNA, or RNA, encoding human papilloma virus major capsid protein L1. During virus trafficking, protein L1 dissociates from the viral DNA and the genomic DNA is released to the host nucleus. The papilloma virion assembly takes place within the cell nucleus. Protein L1 encapsulates the genomic DNA together with protein L2.

As used herein, “tag” refers to a unique molecule that is capable of being specifically recognized by another molecule that binds to the tag. By extension, the term “tagged”, as used herein, refers to the property of a molecule of interest being chemically linked to a tag.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The phrases “% sequence identity,” “percent identity,” or “% identity” refer to the percentage of amino acid residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain.

Nucleic acids, proteins, and/or other compositions described herein may be purified. As used herein, “purified” means separate from the majority of other compounds or entities, and encompasses partially purified or substantially purified. Purity may be denoted by a weight by weight measure and may be determined using a variety of analytical techniques such as but not limited to mass spectrometry, HPLC, etc.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, N.Y.; Wetmur, 1991, Critical Review in Biochem. and Mol. Biol. 26(3/4):227-259; and Owczarzy et al., 2008, Biochemistry, 47: 5336-5353, which are incorporated herein by reference).

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

### EXAMPLES

The following Examples are illustrative and should not be interpreted to limit the scope of the claimed subject matter.

### Example 1

ViroFind workflow comprises, in one example, 131,706 viral probes (8.415 Mbp) with mean coverage of 89.39% of the genome of 561 selected DNA and RNA viruses which infect humans or could cause zoonosis. In one embodiment, DNA and cDNA libraries from clinical samples are sonicated to 150 base pair (bp) fragments, tagged with index adapters and amplified with adapter-specific primers. Libraries are hybridized with the biotinylated viral probes and viral sequences captured using streptavidin coated magnetic beads. The enriched viral DNA and cDNA is amplified by PCR again prior to Next-Gen sequencing with an Illumina NextSeq instrument.

Deep sequencing for virus enriched human samples was performed using the Illumina NextSeq sequencer at Northwestern University NUSeq. Each sample had about 20 million paired-end (PE) 150 bp reads. Next, the results of sequencing were analyzed using the following bioinformatics workflow. Raw de-multiplexed reads from the samples were processed through ViroFind analysis pipeline v2.0. First, reads with overall quality <20 and length <50 bp were discarded using Skewer (‘-q 20-150’) [1]. Reads were further processed to remove low complexity reads and duplicate reads using PRINSEQ++(‘-lc_entropy 90-derep’) [2]. Reads passing these filters were then mapped to human genome reference hg38 using STAR allowing a maximum of 1000 alignments per read (--outFilterMultimapMax 1000) [3].

Paired-end reads that did not map to the human genome were mapped against the set of viral reference genomes downloaded from NCBI using STAR with the filter specified above. Reads mapping to multiple viruses were not used for viral identification. For all identified viruses, breadth and depth of coverage were evaluated using BEDTOOLS genomecov [4]. Sequence Alignment Map (SAM) and its binary version (BAM) files were generated for visualization of the virus-aligned regions [5]. Identified viral regions were matched with gene descriptions from General Feature Format (GFF) files corresponding to the viral references downloaded from NCBI using BEDOPS suite and in-house scripts [6]. Tab-delimited summary files were generated on a per-sample basis to summarize the identified viruses along with their breadth and depth of coverage, coverage normalized for 1M reads and 1000 bp of genome, viral regions and corresponding genes.

PICARD tools was used to mark and remove PCR-derived duplicate reads to generate a set of unique viral reads for variant calling [7]. V-phaser 2 was used to identify viral variants from the virus-aligned reads [8]. Whole genome coverage plots showing per-base coverage across the whole genome for each identified virus were generated using in-house R scripts.

Finally, the reads from identified viruses were assembled into larger contiguous sequences (contigs) using SPAdes de novo assembler [9]. FASTA and FASTQ files were generated for reads mapping to the viruses using SAMTOOLS [5]. Results for multiple samples were visualized using complex heat maps generated with in-house R scripts [10].

### REFERENCES

1. Jiang H, Lei R, Ding S W, Zhu S. Skewer: a fast and accurate adapter trimmer for next-generation sequencing paired-end reads. BMC Bioinformatics. 2014; 15:182. Published 2014 Jun. 12. doi:10.1186/1471-2105-15-182.
2. Cantu V A, Sadural J, Edwards R. PRINSEQ++, a multi-threaded tool for fast and efficient quality control and preprocessing of sequencing datasets. PeerJ. 2019. Preprints 7: e27553v1. doi: 10.7287/peerj.preprints.27553v1.
3. Dobin A, Davis C A, Schlesinger F, et al. STAR: ultrafast universal RNA-seq aligner. Bioinformatics. 2013; 29(1):15-21. doi:10.1093/bioinformatics/bts635.
4. Quinlan A R, Hall I M. BEDTools: a flexible suite of utilities for comparing genomic features. Bioinformatics. 2010; 26(6):841-842. doi:10.1093/bioinformatics/btq033.
5. Li H, Handsaker B, Wysoker A, et al. The Sequence Alignment/Map format and SAMtools. Bioinformatics. 2009; 25(16):2078-2079. doi:10.1093/bioinformatics/btp352.
6. Shane Neph, M. Scott Kuehn, Alex P. Reynolds, et al. BEDOPS: high-performance genomic feature operations. Bioinformatics. 2012; 28(14):1919-1920. doi: 10.1093/bioinformatics/bts277.
7. Picard Tools. http://broadinstitute.github.io/picard.
8. Yang X, Charlebois P, Macalalad A, Henn M R, Zody M C. V-Phaser 2: variant inference for viral populations. BMC Genomics. 2013; 14:674. Published 2013 Oct. 3. doi:10.1186/1471-2164-14-674.
9. Bankevich A, Nurk S, Antipov D, et al. SPAdes: a new genome assembly algorithm and its applications to single-cell sequencing. J Comput Biol. 2012; 19(5):455-477. doi:10.1089/cmb.2012.0021.
10. Gu Z, Eils R, Schlesner M. Complex heatmaps reveal patterns and correlations in multidimensional genomic data. Bioinformatics. 2016; 32(18):2847-2849. doi:10.1093/bioinformatics/btw313.

### Example 2

Aim: Characterize the entire Virome in the putamen, amygdala, cortex and cerebrospinal fluid (CSF) of Parkinson's disease (PD) patients and control subjects using ViroFind.

The inventors will identify any DNA or RNA virus known to infect humans in fresh frozen samples at a location classically affected in PD brains, as well as in their cerebrospinal fluid (CSF) using ViroFind. The inventors will analyze the entire viral genome, characterize viral variants and, potentially, discover novel viruses. Control brain and CSF samples will be from age-matched subjects

Impact on Diagnosis/Treatment of Parkinson's disease: With respect to expected outcomes, the proposed example will allow determination of a possible association between neurotropic viruses and PD and will provide innovative and impactful insight into PD pathogenesis. In addition, these studies will positively impact the management of PD patients by providing potential targets for disease modifying and symptomatic therapeutic interventions.

Next Steps for Development: Data obtained from this example will open new lines of investigations and be used for application for external grants to support scaled-up virological and immunological studies in PD and integrate viromics with genomics and metabolomics.

### Example 3

Alzheimer's disease (AD) is a rampant age-related dementia of unknown etiology characterized by neuronal loss, atrophy, and aggregation of beta amyloid (neuritic plaques) and microtubule associated tau proteins (neurofibrillary tangles) in the brain. While deposition of these proteins is thought to play an important role in the pathogenesis of AD, the presence of these aggregates is not sufficient to cause AD. Both humans and experimental animal models can exhibit one or both of these neuropathological changes without cognitive impairment. Thus, there has been increasing effort to identify other risk factors, including infectious agents that, together with protein aggregation, could fully explain the etiology of this multifactorial disease. In particular, there is evidence that infection by viral pathogens such as herpes simplex (HSV-1 and -2) and human herpesvirus (HHV-6 and -7) could be risk factors for developing AD. However, these viruses are also found in a significant number of healthy individuals and are not consistently enriched in the brains of AD patients (1, 2). Adequately powered and unbiased studies testing for viral genetic material in AD patients and carefully selected control subjects are needed to establish viral infection as a genuine risk factor. In addition, mechanistic experiments investigating the role of these agents in the pathogenesis of AD are needed in order to reconcile infectious etiologies with more established risk factors such as aging and pathological protein aggregation.

The inventors have developed an unbiased target-enrichment deep-sequencing platform for identifying all viruses known to infect humans in clinical tissue samples, including post mortem brains (3). Preliminary data indicate that adeno-associated virus (AAV), and not HSV, HHV, or other known viruses are enriched in the cerebral cortex of AD patients. Unlike viruses previously implicated in AD, AAV is not known to cause human disease. However, the inventors recently reported that the widely used recombinant AAV (rAAV) ablates murine hippocampal adult neurogenesis, which is also markedly diminished in patients with AD (4). In the current example, the inventors will propose to test whether AAV or other viral infections are correlated with the loss of adult neurogenesis and enriched in patients with AD versus control subjects. Secondly, the inventors will investigate the mechanisms by which AAV interacts with tau to attenuate neurogenesis and causes protein aggregation in AD. Specifically the inventors aim to determine whether viral infection correlates with the loss of adult neurogenesis in the human hippocampus and whether these factors are associated with the development of AD.

Recent work demonstrates that hippocampal adult neurogenesis is markedly decreased in the post-mortem brains of AD patients (4), while subjects without dementia demonstrate intact neurogenesis late into adulthood (4-6). In experimental animal models, a number of viruses have been shown to attenuate adult neurogenesis, indicating that this process is sensitive to viral infection (7-9). The inventors' recent experiments indicate that infection with rAAV, which is replication defective and retains only a small portion of the wild-type AAV genome, results in dense ablation of adult neurogenesis in mice (10). The inventors will combine ViroFind with immunolabeling and in situ hybridization (ISH) experiments to test for all 561 viruses known to infect humans and to correlate the inventors' findings to the loss of hippocampal neurogenesis in post-mortem brains from AD patients and aged-matched controls. The inventors hypothesize that AAV infection will correlate with the loss of adult neurogenesis and predict the presence and severity (Braak staging, plaque burden) of AD.

Determine whether tau binds viral genomes and is necessary for rAAV-induced ablation of neurogenesis.

The tau protein has a dozen different isoforms that are purported to have different functions. These include isoforms that translocate to the nucleus to bind DNA during heat-shock-induced damage and other stressors (11, 12). In the mouse dentate gyrus, tau is involved in the regulation of adult neurogenesis (13-16). In addition, the rAAV genome appears to be necessary and sufficient for rAAV-induced toxicity in adult neurogenesis (10) and activates DNA damage response machinery (17). rAAV's 145-base-pair viral genome also contains a putative tau-binding site. The inventors will perform affinity pull-down assays and immunoFISH to test whether nuclear tau binds and colocalizes with rAAV genomic DNA in vitro and in vivo. The inventors will also test if knock out of tau rescues rAAV-induced ablation of neurogenesis. The inventors hypothesize that tau binds the rAAV genome within the nucleus, mediating viral-induced ablation of neurogenesis.

Determine whether rAAV infection results in the production and spread of pathological tau species that contribute to the pathogenesis of AD.

A number of environmental stressors in neurons, including HSV infection, trigger the formation of hyper-phosphorylated tau, a pathological tau species that aggregates and is the major component of neurofibrillary tangles in AD. The inventors propose to determine if rAAV infection can induce the formation of pathological tau species in human IPS-cell derived and mouse models of AD and controls. The inventors hypothesize that rAAV infection will generate hyper-phosphorylated tau in neurons from both AD models and controls to different degrees, and upon seeding with tau fibrils will lead to enhanced tau deposition and spreading to adjacent brain regions compared to seeding alone.

### Example 4

Alzheimer's disease (AD) is a progressive age-related dementia, accounting for 60% to 80% of all dementia diagnoses. AD is the 6th leading cause of death and the number one cause of morbidity in the United States. The incidence of AD is expected to climb dramatically as the population ages, affecting 14M Americans by 2050. Despite more than a century of investigation, the etiology of AD is unknown and there exist no disease modifying therapies for this rampant and devastating illness.

AD pathology is characterized by the presence of extracellular amyloid beta (Aβ) plaques and intracellular neurofibrillary tangles (NFTs) consisting of hyper-phosphorylated tau. Triggers that induce formation of Aβ and pathological tau species and how this process leads to neuronal loss in AD are not understood. Moreover, deposits of aggregated Aβ and tau can be found in cognitively normal subjects (18-23) and multiple clinical trials testing treatments that substantially reduce the accumulation of these proteins show no effect on the progression or symptoms of AD (24-27). Questions about the causative role of Aβ and tau have motivated investigation of “multi-hit” hypotheses (28) and alternative risk factors for developing AD.

Adult Neurogenesis is Attenuated in Alzheimer's disease (AD). Whether adult neurogenesis is present in the hippocampal dentate gyrus (DG) of aged humans and could be affected in neurodegenerative diseases remains a matter of debate (29-31). Studies suggests that the severity and perhaps even the development of AD is related to the loss of adult neurogenesis (4, 32-34). Indeed, adult neurogenesis is markedly decreased in the post-mortem brains of AD patients, even before NFTs can be identified in the hippocampus (4). In contrast, people without dementia demonstrate intact neurogenesis late into adulthood (4-6, 35), including healthy subjects who are non-demented but exhibit AD neuropathology (NDAN) at autopsy (36). Consistent with this idea, memantine improves cognition in patients with AD and has been shown to sharply enhance adult neurogenesis in animals (37). In addition, attenuation of adult neurogenesis has been identified in animal models of AD (33, 38, 39). Moreover, amyloid beta precursor protein (APP), tau, and presenilins are expressed in developing dentate granule cells (DGCs) and regulate neurogenesis in animals (33, 40-46). Whether newborn DGCs are sentinel neurons (“canaries in the coal mine”) for degenerative changes in AD or whether there is a causal link between attenuation of neurogenesis and the development of AD is not known.

Viral Infections Play a Role in Adult Neurogenesis and AD. The ability of numerous viruses to kill neural progenitor cells (NPCs), attenuate neurogenesis, and cause microcephaly and other neurodevelopmental disorders is well-known (47-56). Likewise, human immunodeficiency virus (HIV), herpes simplex virus (HSV), Zika virus, and recombinant adeno-associated virus (rAAV) attenuate adult hippocampal neurogenesis, indicating that this process is also sensitive to viral infection (8-10, 54, 57). Chronic HIV infection causes an age-related dementia with amyloid plaque and NFT pathology similar to AD (58, 59). In addition, HSV-1 infection results in Aβ accumulation and reduces hippocampal NPC proliferation in vitro, which is blocked by β- and γ-secretase inhibitors (57). Also, reactivated HSV-1 infection decreases proliferation of newborn dentate granule cells (DGCs) in the mouse dentate gyrus (DG), which was not observed in amyloid-β precursor protein (APP) knockout mice. Lastly, the inventors recently discovered that rAAV eliminates dividing NPCs in both the neurogenic niche of the adult mouse DG in vivo and mouse NPCs in vitro (10).

Viral infections have also been implicated in the pathogenesis of AD. The idea originated in the 1980s (60), with subsequent studies confirming 1) the presence of HSV DNA in brains from AD patients (61-63), particularly in patients carrying apolipoprotein 4E (APOE4) variant (2, 64, 65), and 2) the enrichment of viral DNA in amyloid plaques (66). Also, HSV and other herpesviruses (cytomegalovirus, humanherpes virus [HHV]) can induce the formation of Aβ by neurons and other cells (67-69), which is suspected to be a defense mechanism against viral infection (68). Despite over 3 decades of investigating herpesviruses in the pathogenesis of AD (70-72), the clinical significance of these findings remain unclear due to the equally high incidence of these viral infections in the healthy general population (73).

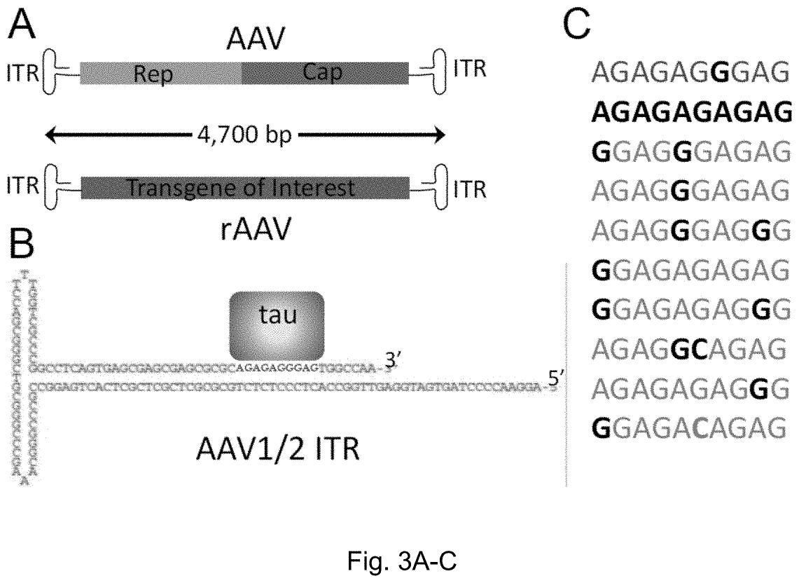

AAV as a potential link for impaired neurogenesis and the development of AD. Wild-type adeno-associated virus (AAV) is a replication defective, non-enveloped single stranded DNA parvovirus with no known pathogenicity (74-76). AAV was originally isolated in the 1960s from adenovirus stocks, and was thought be a precursor or contaminant. It was later identified as a distinct virus that requires co-infection from a helper virus, such as adenovirus, to enter the lytic phase. AAV contains a 4.7 kilobase (kb) genome that includes the rep and cap genes, and a pair of palindromic 145 base pair (bp) inverted terminal repeat (ITR) DNA segments (FIG. 3A-C). The rep gene codes for 4 multifunctional proteins (Rep78, Rep68, Rep52, and Rep40), which are necessary for viral DNA replication, integration into the host chromosome, and packaging of the ITR-flanked viral genome into the capsid. Co-infection with adenovirus, herpesvirus, or human papilloma virus (HPV) is required for the replication of AAV. In the absence of a helper virus, AAV can produce a latent infection in which the viral genome is maintained in an episomal form, or inserts into the host genome and persists in infected cells. AAV's primary route of transmission is via respiratory infection. However, AAV has been identified in a variety of tissues, including isolated occurrences in the brain (77). Despite 30-80% of individuals testing positive for antibodies to AAV, it has not been identified to cause any known human disease.

During viral production, the rep and cap genes can be supplied in trans to create additional space for transgenes, resulting in the widely used recombinant AAV (rAAV) vector. The only remnant of the AAV genome within rAAV are the ITRs, which are essential for packaging the transgene into the capsid and provide the initiation site for the host DNA polymerase to complete second strand synthesis (78). While there are 12 known human serotypes of AAV, the majority of rAAVs manufactured for experimental and clinical applications utilize the AAV2 ITR. This ITR can be packaged into most capsid serotypes during production, including a number of engineered designer capsids (79), expanding the utility of the recombinant vector. rAAV's minimal genome and limited immunogenicity and toxicity have made it the human gene therapy vehicle of choice and has been tested in over 100 clinical trials, including the two FDA approved rAAV therapies (80-82). Despite this safety profile, the inventors demonstrate that rAAV induces toxicity in the neurogenic niche of the adult hippocampus and could be important for understanding the relationship between impaired adult neurogenesis and the development of AD.

In order to investigate the role of viral infection in the pathogenesis of AD, the current example relies on 3 areas of innovation. Perform the 1st unbiased search for all known human viruses in brain tissue of AD patients. Although there exists compelling evidence for the role of viral infections in the pathogenesis of AD, targeted searches for HSV, HHV, and other viruses have not identified an increased incidence of viral infection in patients with AD compared to aged-matched controls. The inventors have developed an unbiased target-enrichment deep-sequencing platform, ViroFind, for identifying all viruses known to infect humans in clinical tissue samples, including post mortem brains (3). By implementing the ViroFind pipeline in the hippocampus (HC) and entorhinal cortex (EC) of AD patients and aged-matched controls, the inventors aim to investigate whether viral infection is a genuine risk factor for AD.

Investigate the novel hypothesis that AAV is a causative agent for AD. While there exist isolated reports of toxicity resulting from infection by AAV and its recombinant form, AAV has never been conclusively demonstrated to cause disease in humans. The inventors' preliminary data suggests that AAV is enriched in the brains of AD patients, which if substantiated would establish AAV infections as a novel risk factor for the development of AD. The inventors will also perform the first mechanistic studies investigating how AAV infection results in AD pathology.

Investigate the novel hypothesis that viral infection is responsible for impaired adult neurogenesis in AD patients. Studies demonstrate that hippocampal adult neurogenesis is markedly decreased in the post-mortem brains of AD patients compared to controls (4). The mechanism underlying this phenomenon is unknown. The inventors propose to perform the first studies in humans correlating the presence of virus with attenuation of adult hippocampal neurogenesis.

Experimental Approach

Determine whether viral infection correlates with the loss of adult neurogenesis in the human hippocampus and whether these factors are associated with the development of AD.

Hypothesis: The extent of AAV infection in the human HC and EC will correlate with the loss of adult neurogenesis and predict the presence and severity (Braak staging, plaque burden) of AD.

Rationale: The rationale for this example is built upon two important observations: 1) Hippocampal adult neurogenesis is markedly attenuated in the post-mortem brains of AD patients (4, 36, 83), and is already evident at Braak stage I, before NFTs can be identified in the DG, and diminishes with advancing AD pathology (4). 2) Adult neurogenesis is exquisitely sensitive to viral infections (8-10, 54, 57). The inventors' preliminary experiments provide the first clues that AAV infection may be the common factor underlying these observations and an important risk factor for the development of AD. Specifically, the inventors found evidence of AAV infection in the supramarginal cortex of 3/10 AD patients versus 0/10 control subjects (see Preliminary Data below). In addition, the inventors discovered that recombinant AAV ablates adult neurogenesis in the murine dentate gyrus in a dose dependent fashion. If it is true that AAV infection is a risk factor for both of these processes, then the presence of AAV infection should predict both the loss of adult hippocampal neurogenesis and the existence and severity of AD pathology in the human brain. In this example, the inventors will use ViroFind in frozen postmortem tissue from the HC and EC to test for genetic material from all 561 viruses known to infect humans. Findings of viral genomic material will be verified by performing in situ hybridization (ISH) in these brain regions. Immunohistochemistry in paraffin embedded formalin fixed tissue from the contralateral HC and EC in the same subjects will be performed to quantify the extent of hippocampal neurogenesis. The extent of viral genetic material and hippocampal neurogenesis will be correlated to each other and to the presence and severity of NFT and plaque pathology in AD patients and aged-matched controls, establishing whether viral infection is a genuine risk factor for loss of neurogenesis and the development of AD.

Preliminary Data

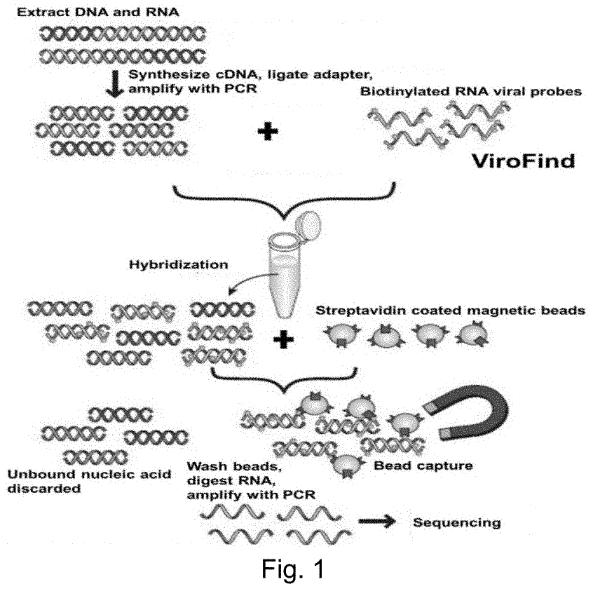

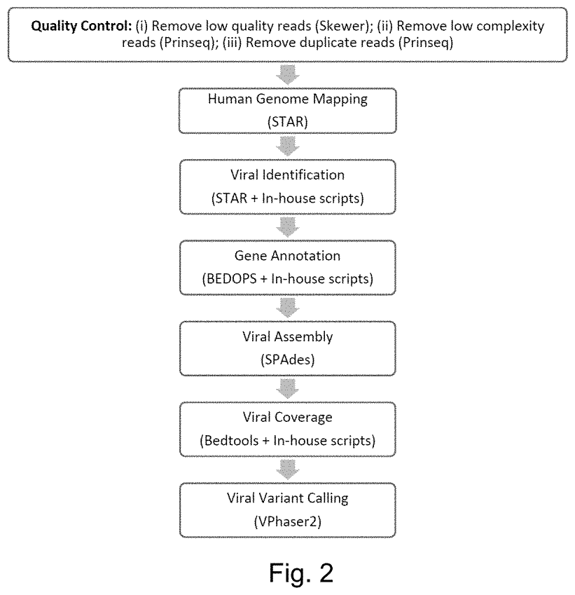

ViroFind identifies AAV as a potential risk factor for AD. The Koralnik Lab has developed a target-enrichment platform for virus detection and discovery in clinical samples (FIG. 1) including postmortem brain (3) and heart (84) tissue. ViroFind has been used to detect and analyze all viral populations in the brain of 5 patients with progressive multifocal leukoencephalopathy (PML) and of 18 control subjects with no known neurological disease (3). These studies demonstrate that by using pull-down techniques to isolate viral DNA and RNA from human samples prior to sequencing, ViroFind can enrich viral sequences present in clinical samples up to 127-fold and increase signal to noise compared to deep sequencing alone. Using this approach, the inventors discovered complex polyoma virus JC populations that exhibited a high degree of genetic divergence in the brain samples with PML. The inventors also detected sparse human herpes virus 6B (HHV6B) sequences in 11 brain samples out of the 18 (61.1%) control subjects (3).

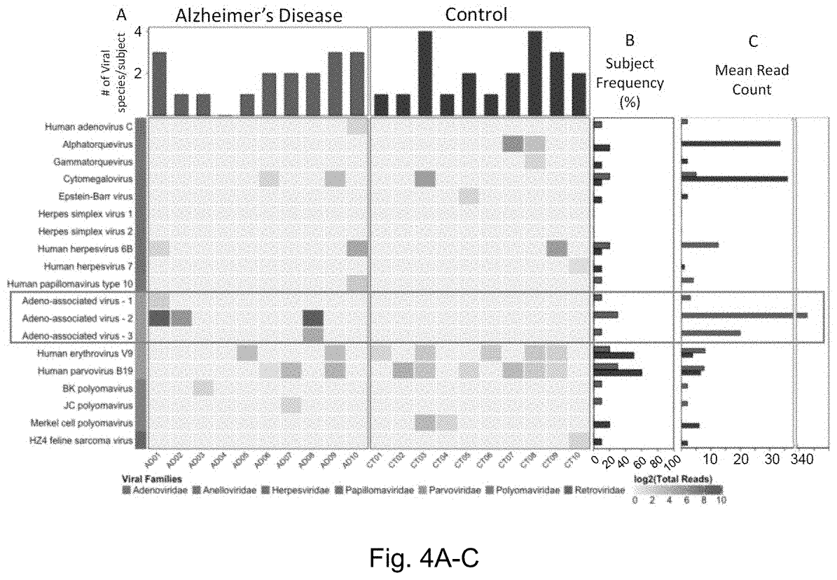

The inventors have used the ViroFind platform to test the supramarginal gyms from 10 AD patients and 10 sex and age matched control subjects for viruses. The results from 17 different viruses from 7 viral families are shown in FIG. 4A-C. Among the 561 viruses tested, only AAV was enriched in the brains of AD patients, with 3/10 subjects exhibiting AAV in the cortex versus 0/10 control subjects. Importantly, AAV infections were associated with a high unique read count that is at least 10-fold higher than all other viruses tested. Because ViroFind measures both DNA and cDNA, high read counts generally reflect either 1) viral production of mRNA transcripts during expression of viral genes or 2) multiple viral genomes present (in multiple cells) throughout the tissue. The inventors performed polymerase chain reaction (PCR) for the AAV rep gene in the same samples, which confirmed the presence of AAV DNA. Reverse transcriptase (RT) PCR for the AAV rep gene showed no active transcription (data not shown). In addition, using the methods of the current disclosure biases the mean unique read count toward viruses with larger genomes that produce more unique DNA/cDNA fragments than smaller genome viruses such as AAV. To account for this, the inventors normalized the mean read count to the length of the viral genome, which for AAV2 yields a normalized mean read count >49 times larger than all other viruses tested. Taken together, these experiments indicate a pervasive latent AAV infection within this region of cortex in these AD patients. These findings warrant further investigation of AAV infection in the HC and EC, where AD pathology is observed earliest.

An intriguing aspect of the data that highlights the power of the ViroFind approach, is that of the known AAV helper viruses, HHV6B was identified in only one of the three patients positive for AAV. The inventors are currently developing explanations for this finding, including identifying possible helper viruses for AAV that have not been previously reported. There are also reports of helper-virus-free AAV replication in the setting of cellular genomic stress and DNA damage (85), which also have been implicated in the pathophysiology of AD. These findings of AAV infection in AD patients without evidence of obligatory co-infection by a helper virus provides additional support that a replication defective rAAV is a valid model system for studying the role of AAV toxicity in the pathogenesis of AD.

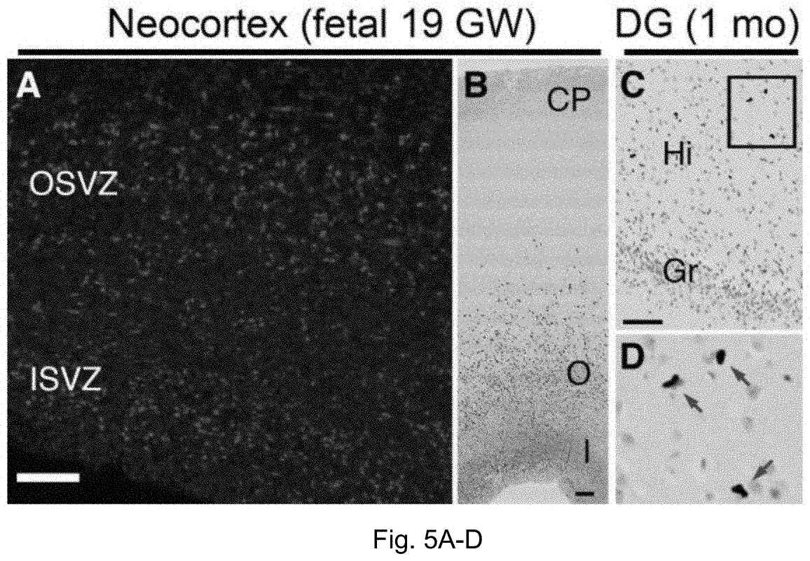

Tbr2 as a marker of adult hippocampal neurogenesis in humans. Studies in adult rodents demonstrate that Tbr2 is specifically expressed in transiently amplifying cells in the subgranular zone (SGZ) of the dentate gyrus (DG) (86-88) and throughout the developing cortex of mammals, including humans (89). For this project, sections of formalin-fixed, paraffin-embedded (FFPE) sections of human DG will be studied by immunohistochemistry (IHC) to detect Tbr2. To confirm that the inventors can detect Tbr2 in FFPE human brain, the inventors first studied sections of fetal human neocortex, which is known to express Tbr2 in the outer subventricular zone (OSVZ) and inner subventricular zone (ISVZ), as shown previously (85). Tbr2 was detected in the appropriate pattern by immunofluorescence (FIG. 5A) and by IHC using a color reaction (FIG. 5B) on the Ventana processor (see Experimental Design and Methods), confirming the sensitivity and specificity of the inventors' methods. Next, the inventors studied DG from early postnatal humans (up to 2 years old), where Tbr2 was detected predominantly in the hilus (Hi), without any obvious compartmentalization in the SGZ (FIGS. 5C&D). Interestingly, new neurons have also been described mainly in the hilus, without SGZ formation, in a previous study of human DG neurogenesis (29). Having verified the inventors' ability to detect Tbr2 in FFPE sections of postnatal human brain, the inventors' next step will be to study a series of older humans and patients with AD.

Experimental Design and Methods:

Perform ViroFind in the HC and EC of patients with AD with different Braak staging and aged-matched controls. The ViroFind pipeline (3) will be performed on brain tissue (HC and EC) from 35 AD patients (Braak I-VI) and 17 age and sex matched controls without history of dementia or other brain disease (104 total locations). DNA and RNA will be extracted from fresh frozen brain tissue using a spin-column method (90) and used to create complementary DNA (cDNA) libraries. DNA and cDNA will be sonicated to 150-200 bp fragments, followed by ligation of the 3′ ends to adapter molecules. Adapters contain known sequences that allow index tagging for each sample. In addition, adapters contain primers that are necessary for PCR amplification. Subsequently, the DNA and cDNA fragments with adapters are incubated with the biotinylated viral RNA probes to allow hybridization. Streptavidin coated magnetic beads are used to pull-down and isolate hybridized sequences away from nucleic acids that are not complimentary to the RNA probes. Nucleic acids which do not bind to RNA probes are removed by 7 wash cycles. The remaining enriched DNA/cDNA is amplified with primers to the adapters, followed by paired-end sequencing with NextSeq (Illumina).

The inventors will perform a quality check on the raw sequencing data and discard reads that do not pass standard quality filters (90). The unique sequence of each index tag allows each read to be assigned to a single clinical sample (de-multiplexing). Lastly, the inventors will computationally “trim” the adaptor sequences from all reads. The inventors will align the inventors' reads against the human genome and the inventors will discard any reads that align to the human genome. The inventors then align the remaining reads against the NCBI dataset of all viral genomes and detect and analyze viral sequences in the sequencing data using previously published methods (91). Additional analysis will be performed to determine viral integration within the host genome.

Determine if the loss of immature neuronal markers, Tbr2 and DCX, is correlated with the presence of virus and pathological markers of Alzheimer's disease (AD). All brain tissue will be processed by the UCSD Pathology Histologic Biomarkers Core. Tissue sections will be cut from blocks of formalin-fixed paraffin embedded HC and EC. Four micron tissue sections will be stained with antibodies for neurogenesis markers Tbr2 (Hevner Lab, (86)) and DCX (Atlas HPA036121, Santa Cruz sc-271390, Millipore AB2253) and for dividing cells positive for PCNA (Genetex GTX100539, Santa Cruz sc-25280). All IHC and ISH probes will be optimized by extensively testing various conditions (antibody dilution, antigen retrieval protocol, staining time), which is rapidly accomplished using the high-throughput Ventana Discovery Ultra (Roche Diagnostics). Antigen retrieval will be performed using cell conditioning solution (CC1, Roche Diagnostics) for 24-40 minutes at 95 C (or Protease 2 (Roche) for 12 min). The primary antibodies will be incubated on sections for 1 h at 37 C and visualized with 3,3′ diaminobenzidine (DAB) using the UltraMap system (Roche Diagnostics) followed by hematoxylin counterstain. Slides are rinsed, dehydrated with alcohol and xylene, cover-slipped, and analyzed by conventional light microscopy.

All brains at the UCSD ADRC are assessed according to the National Alzheimer's Coordination Center (NACC) Neuropathology Data Form. This includes staining for beta amyloid (AB69 antibody courtesy of Edward Koo) and hyper-phosphorylated tau (PHF1 antibody, courtesy of Peter Davies) using the Ventana system. The amount of plaques and tangles will be quantified using the Thal Phase (92) and Braak (NFT) Staging (93), respectively, and correlated to the presence or absence of neurogenesis markers (number of positive cells per mm3) and viral burden (unnormalized and normalized read count) described above.

Confirm the presence of the viral genomes identified by ViroFind and the presence or loss of immunohistological neurogenesis markers using ISH.

The presence and location of viral DNA/RNA, and Tbr2 and DCX mRNA within the human HC will detected using the RNAscope and DNAscope system (Advanced Cell Diagnostics), an in situ hybridization method that permits signal amplification of cellular and viral mRNA transcripts as well as viral DNA (94, 95). Tissue will be drop-fixed in neutral-buffered formalin and processed and embedded in paraffin. Five 5 μm tissue sections will be collected in RNase-fee manner and dried at room temperature overnight. Again, using the Ventana processor, slides will be baked for ˜30 min at 60 degrees, de-paraffinized, and subjected to antigen retrieval. Slides will be treated with protease with two sequential incubations at 65 and 75 degrees for 12 min each to enhance probe penetration. Custom nucleic acid probe sets are provided for each target by the manufacturer based on >1 kb of the target sequence. Following amplification steps resulting in a large number of horseradish peroxidase molecules per mRNA or DNA molecule, the probe will be visualized by incubation with 3,3′-Diaminobenzidine (DAB). Sections will be counterstained with hematoxylin and analyzed by light microscopy.

Anticipated results, potential pitfalls, and alternative approaches: The inventors expect to identify all of the DNA or RNA viruses present in AD and control brain samples. These could be known viruses, variants of known viruses harboring deletions or mutations, or potentially yet unknown viruses that contain homologous genomic regions. The inventors expect that the virome in AD brains will be qualitatively and quantitatively different than from control brain samples. Based on the inventors' preliminary ViroFind studies, the inventors expect that AAV will be enriched in the brains of AD patients. The inventors also predict that the amount of AAV present in the HC will correlate to the extent of loss of adult neurogenesis observed in this brain region, where the inventors' preliminary experiments in rodents suggest that the loss of the Tbr2 marker will more sensitive than Doublecortin (DCX) for mild cases. Based on these predictions and studies in the literature (4), both metrics of viral involvement and neurogenesis should correlate with the severity of AD pathology, namely Braak staging and amyloid plaque burden (see caveats below).

The subgranular zone (SGZ) of the dentate gyrus (DG) is highly vascularized, and the blood brain barrier in this region is leaky during postnatal development and in adult mice after experiencing systemic release of cytokines and growth factors resulting from contralateral cerebral vascular occlusion (96, 97). Therefore, it is possible that the neurogenic niche is particularly susceptible to infection from systemic AAV, which is only 22 nm in diameter. This implies that testing of the HC and adjacent EC tissue could reveal a higher rate of AAV infection than estimated by the inventors' preliminary data obtained in the superior marginal gyms. Alternatively, it is possible that by the time AD pathology evident, the disease is too advanced and infected neurons in these regions have already been destroyed. In this case, the inventors will repeat ViroFind experiments using other areas of the cortex with lower AD burden.

Although the inventors did not find evidence of infection of helper viruses in the inventors' preliminary experiments, it is possible that infection from no single viral species can account for the development of AD. Instead, it might be the interaction between two or more viruses that is predictive of AD in the inventors' samples. Because ViroFind allows for an unbiased search of the entire virome, the inventors are in an excellent position to observe such interactions as well as the existence of multiple independent viral infections that serve as risk factors for the disease using the methods disclosed herein.

Determine whether tau binds viral genomes and is necessary for rAAV-induced ablation of neurogenesis. Hypothesis: Tau binds the AAV genome within the nucleus and mediates AAV-induced ablation of adult neurogenesis. Rationale: Numerous studies show that tau is expressed in NPCs and developing neurons (15, 98-101) and is involved in the regulation of neurogenesis during development and adulthood (14, 16, 43-46, 102). Also, chromosomal abnormalities at 17q21.3, which contains the MAPT gene, is associated with microcephaly (103, 104). Various mouse models, each expressing different human tau variants, show either enhanced (45)(16) or attenuated (46) proliferation in the adult mouse DG. In addition, tau promotes survival of newborn DGCs in response to enrichment (13) and mediates cell-death during stress (13, 14), where it translocates to the nucleus (105).

Nuclear tau was first identified over 30 years ago in the brains of AD patients (106) and later in the rodent brain (105, 107, 108). Experiments indicate that tau strongly binds DNA and other nucleic acids within the nucleus of neurons (105, 109, 110). While the role of DNA-binding of tau is unknown, studies suggest that nuclear tau stabilizes DNA in the presence of cellular stress and is involved in DNA damage response (DDR) (11, 108, 111, 112). A systematic study isolating tau DNA binding sites in mouse neurons identified a family of 10 bp repetitive AG-rich motifs (113). Remarkably, the inventors identified a putative 10 bp AG-rich tau-binding motif on the stem region of the AAV1/2 ITR upstream of the P5 promoter (FIG. 3A-C). Tau has been shown to bind ribosomal DNA (rDNA) repeats near promotor regions and recruit upstream binding factor (UBTF) to increase rDNA transcription (12). Therefore, it is possible that AAV evolved to recruit tau and other DDR machinery to enhance transcription of the viral genome (17, 114-116). However, during neurogenesis, activation of DDR can induce apoptosis and mutations in DDR pathways, including in the tau gene (103, 104), are associated with microcephaly (117-120).

Preliminary data indicate that rAAV induces apoptosis in hippocampal NPCs within 12-18 hours of viral injection and that the ITR DNA is sufficient and necessary to induce this toxicity. These experiments also indicate that this toxicity is sequence specific, where ITR DNA is significantly more toxic than scrambled ITR DNA. Previous studies indicate that it takes approximately 6 to 8 hours for AAV virus to enter the nucleus (66). This limited time window suggests that the proteins that bind the rAAV genome and induce cell death are normally present within the cell and are not newly transcribed or translated. The inventors hypothesize that in response to AAV infection, tau binds the stem region of the ITR within the nucleus, and mediates viral toxicity in NPCs and ablation of adult neurogenesis. In this aim, the inventors will perform affinity pull-down assays to test if AAV ITR DNA binds to tau within NPCs from mouse and human AD models. The inventors will then use immune fluorescence in situ hybridization (immunoFISH) to confirm that tau and the AAV genome co-localize within NPCs. Finally, the inventors will test if tau is necessary for AAV-induced ablation of neurogenesis, by testing the effects of the virus on neurogenesis in tau knockout (ko) mice.

Preliminary Data:

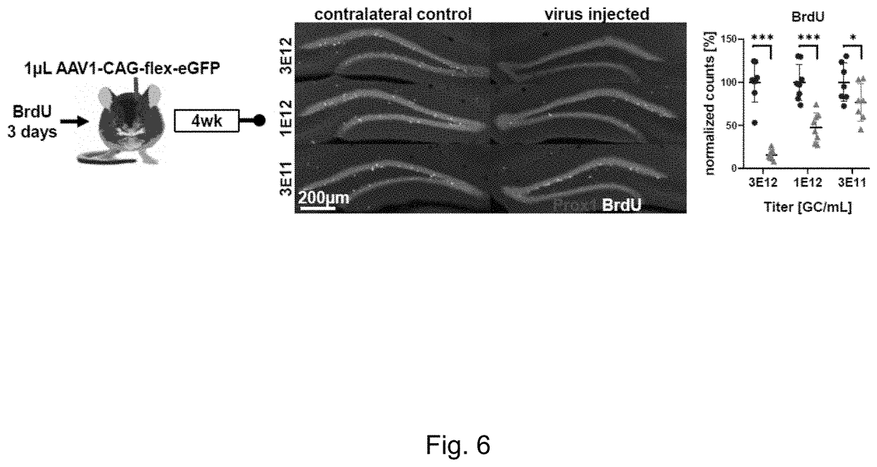

rAAV eliminates adult-born DGCs in a dose-dependent manner. Motivated by the inventors' own efforts to study the function of adult neurogenesis and the DG in learning and memory, the inventors found that delivery of fluorescent proteins using rAAV resulted in ablation of adult neurogenesis (10). This effect was robust regardless of purification method (iodixanol, CsCl), capsid serotype (AAV1, AAV8, AAV9), promoter (CAG, Syn, CaMKIIa), and protein expression (GFP, jRGECO1a, mCherry, data not shown, (10)). To quantify this effect, the inventors injected a minimally expressing cre-recombinase-dependent virus (AAV1-CAG-flex-eGFP, Addgene #51502) in non-cre-expressing wild type C57BL/6J mice to mitigate any contributions from toxicity that might be attributed to protein expression. The inventors first measured the effect of viral titer on rAAV-induced cell loss. The inventors labeled dividing adult-born DGCs for 3 days with BrdU and then injected 1 μL of either 3 E12 gc/mL, 1 E12 gc/mL, or 3 E11 gc/mL rAAV (FIG. 6). Cell loss increased with increasing viral titer. A nearly complete ablation of BrdU+ cells was seen with 3 E12 gc/mL rAAV (−84.3%±6.7%, p<0.001), whereas partial ablation of BrdU+ cells resulted from the injection of 1 E12 gc/mL rAAV (−52.1%±6.7%, p<0.001), and a small reduction of adult neurogenesis resulted from injection of 3E11 gc/mL rAAV (−23.4%±7.2%, p<0.05); all results reported as change relative to non-injected contralateral DG+/−standard error of the mean difference, significance reported as: * p<0.05, ** p<0.01, *** p<0.001, unless stated otherwise.

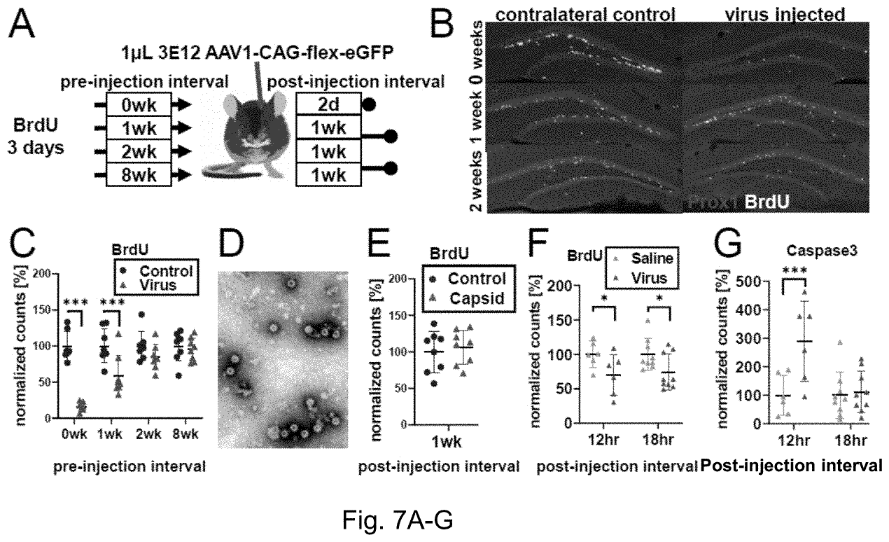

Next, the inventors investigated whether cell survival depended on the age of the cells at the time of injection (Ftreatment×time(3,27)=29.0, p<0.001; FIG. 7A-G). Cells that were 2 days old and younger were almost completely eliminated within 48 hours (−83.9%±6.7%, p<0.001). Cells that were 7-9 days old were partially protected (−41.3%±6.3%, p<0.001), whereas cells that were 14-16 days old were largely protected (−15.4%±6.3%, n.s.).

Mature DGCs approximately 8 weeks old also did not demonstrate significant loss (4.5%±6.3%, n.s.; FIG. 7C). To determine the effect of removing the viral genome, the inventors injected empty AAV viral capsid (3.7 E13 capsids/mL) into the DG (FIG. 7E), which has been shown to penetrate the cell, similar to rAAV with intact genome (121). At 1 week post-injection, empty capsid had no effect on 2-day old BrdU+ cells (6.2%±3.2%, n.s.). Given the rapid ablation of neurogenesis, the inventors designed an acute time-course experiment to visualize cells in the process of dying. Following labeling with BrdU, animals were injected with 1 μL of 3E12 gc/ml rAAV into one dorsal DG and 1 μL saline into the contralateral DG to control for the acute effect of surgery- and injection-induced inflammation and tissue damage. rAAV-injected DGs had a modest decrease in BrdU+ cells at 12 and 18 hours relative to saline-injected control (Ftreatment(1,13)=13.9, p<0.01; interaction with time n.s. FIG. 7F). Cell loss was accompanied by an increased number of Caspase-3+ apoptotic cells at 12 hours (Ftreatment×time(1,13)=21.2, p<0.001; 12 h treatment: +188.6%±29.8%, p<0.001; 18 h treatment: 11.7±24.3%, n.s.; FIG. 7G).

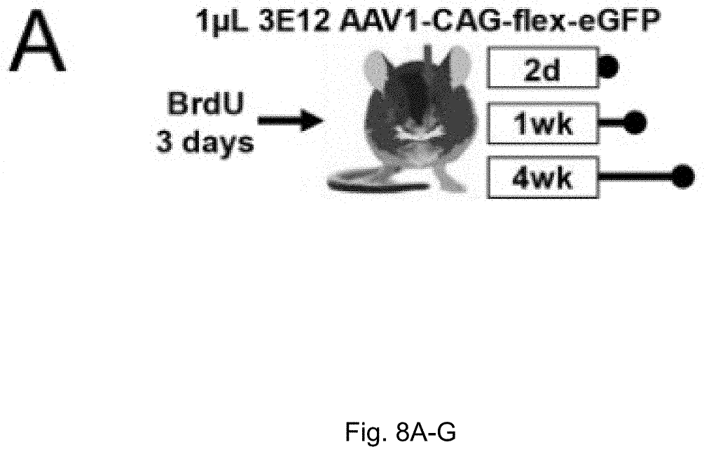

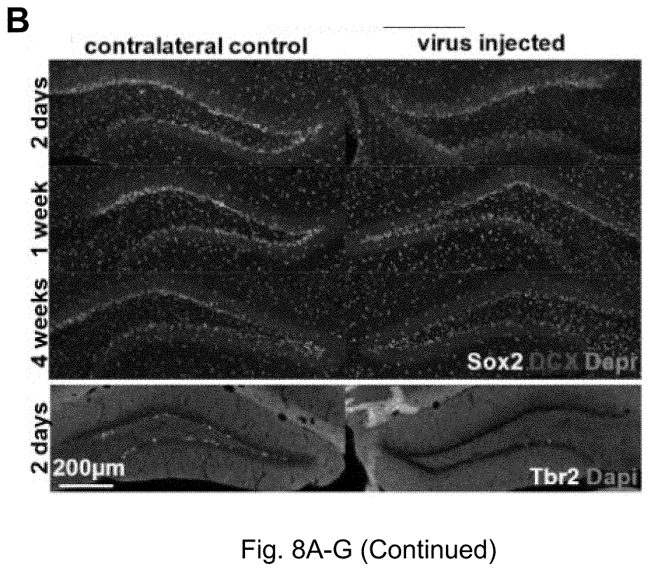

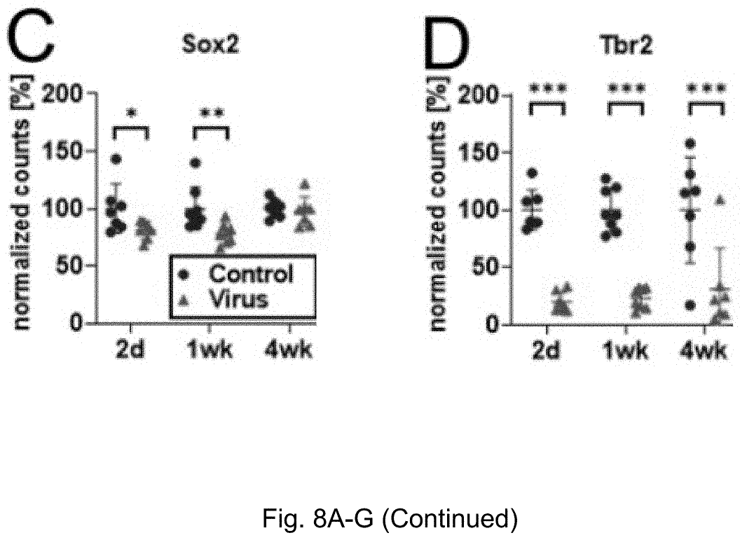

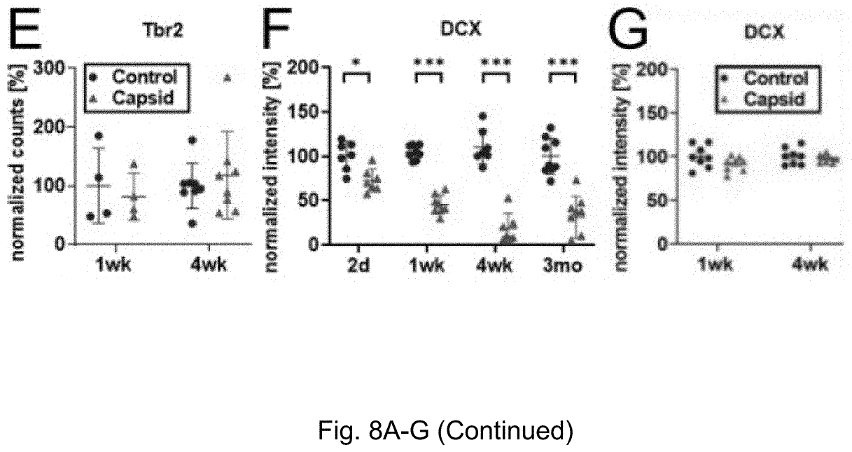

NPC developmental stage determines susceptibility to rAAV-induced cell loss. After determining the response of newborn DGCs to rAAV based on post-mitotic age, the inventors determined which population of NPCs was susceptible to rAAV toxicity. To accomplish this, the inventors varied the post-injection interval and measured canonical early (Sox2), middle (Tbr2), and late (DCX) histological markers associated with adult-born DGC development. Mice were unilaterally injected with 1 μL of 3 E12 gc/mL rAAV and sacrificed at 2 days, 1 week, or 4 weeks post-injection (FIG. 8A-G). The number of Sox2+ cells within the SGZ was modestly decreased (Ftreatment(1,19)=15.5, p<0.001; interaction with time n.s.). In contrast, Tbr2+ intermediate progenitor cells were almost entirely lost and did not show signs of recovery by 4 weeks post-injection (Ftreatment(1,19)=129.2, p<0.001; interaction with time n.s.). Expression of the late premitotic and immature neuronal marker DCX showed a progressive decline until near complete loss at 4 weeks post-injection (Ftreatment×time(3,27)=12.8, p<0.001; 2 days: −27.7%±9.0%, p<0.01; 1 week: −58.7%±8.4%, p<0.001; 4 weeks: −92.0%±9.0%, p<0.001;) and did not show signs of recovery 3 months post-injection (−68.7%±8.0%, p<0.001; FIG. 7F). These findings suggest that the largely quiescent Sox2+ pool remains mostly intact. Instead, the rapid loss of proliferating Tbr2+ NPCs drives much of the rAAV-induced toxicity, including the progressive loss of the DCX+ population that is observed as they differentiate into mature neurons and decline in number over time.

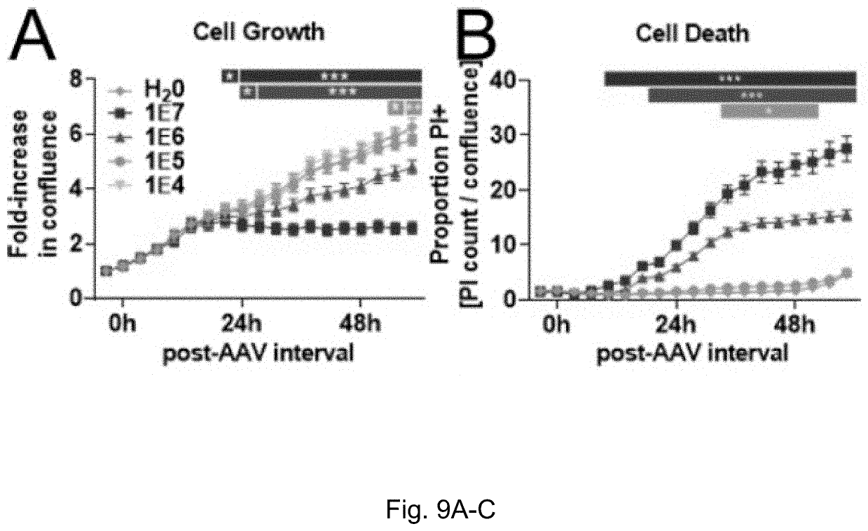

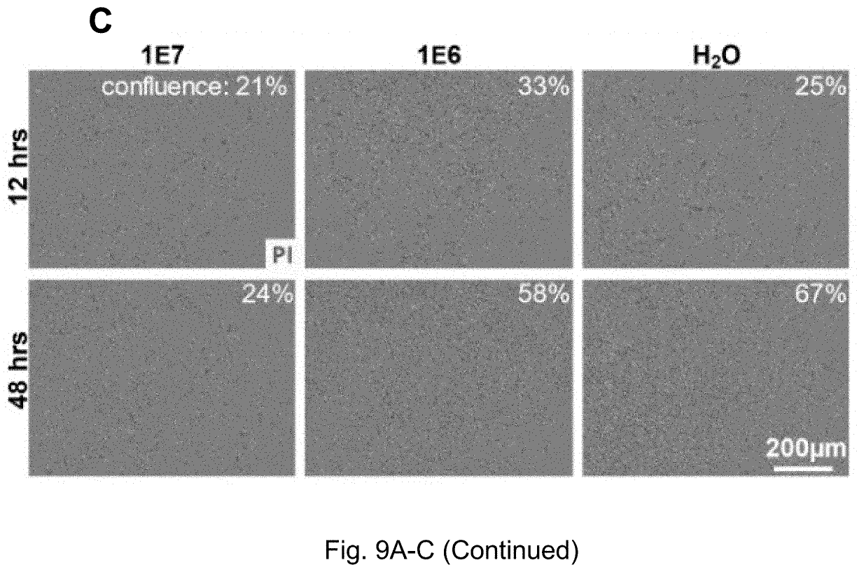

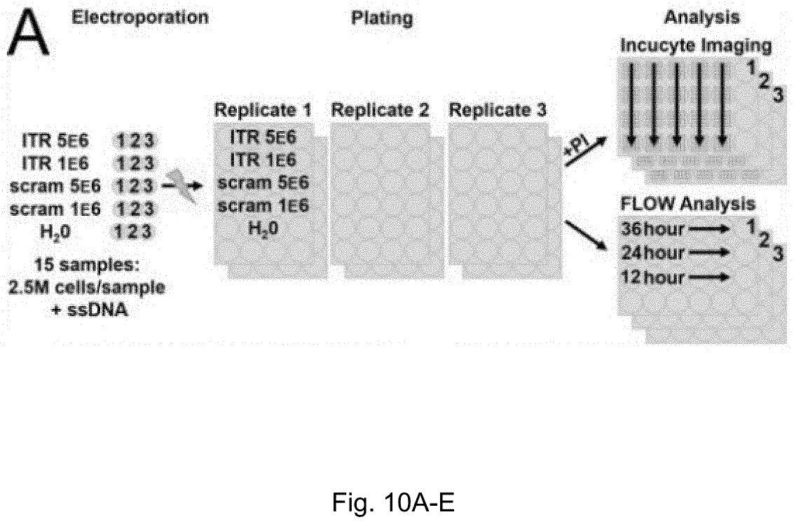

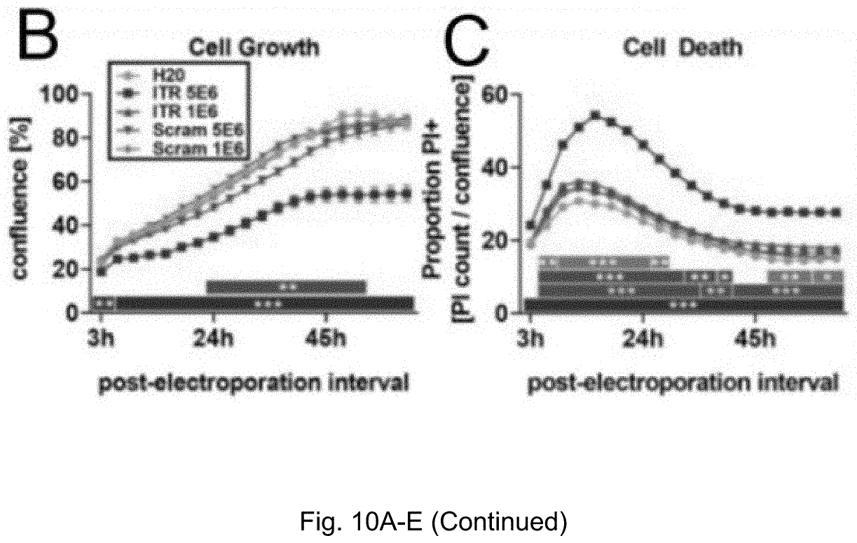

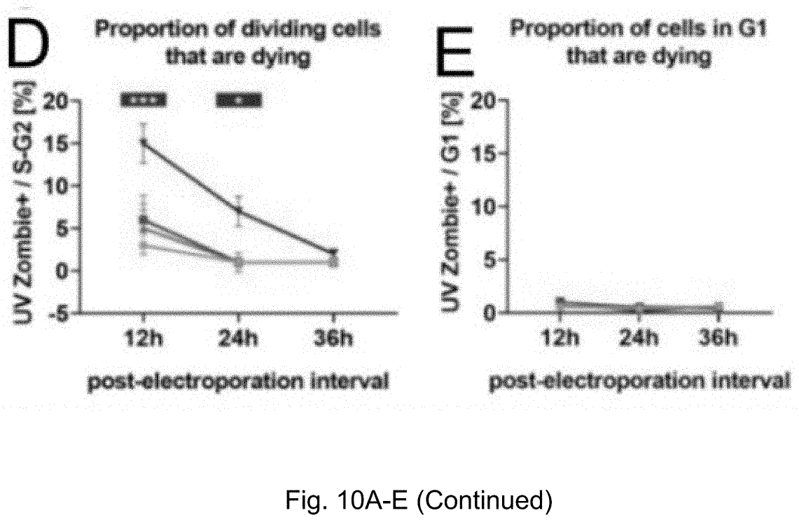

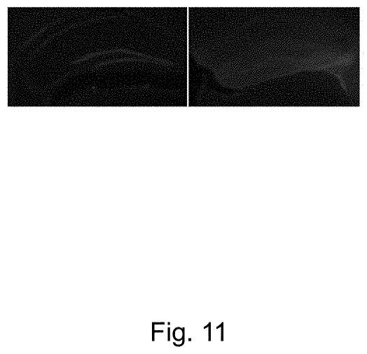

rAAV-induced toxicity is cell-autonomous and can be reproduced in vitro. The inventors investigated whether rAAV-induced cell loss could be explained by inflammation. In contrast to the rapid loss of NPCs (FIG. 7A-G), expression of the microglial marker, Iba1, and the astrocyte marker GFAP was unchanged in the SGZ and hilus at 2 days post-injection and did not peak until 4 weeks post-injection ((10), data not shown). To further explore whether rAAV-mediated toxicity is cell-autonomous, the inventors developed an in vitro assay to study rAAV-induced elimination of NPCs (FIG. 9A-C). Primary mouse NPCs (122) were administered rAAV with a multiplicity of infection (MOI) of 1 E4 TO 1 E7 or H₂O control, and chronically imaged to measure cell survival and proliferation. Dose-dependent inhibition of NPC proliferation was most profound with an MOI of 1 E7 and decreased with reduction in MOI, where infections with 1 E5 and 1 E4 MOI were nearly indistinguishable from H₂O control (FIG. 9A). Cell death, visualized by permeability to propidium iodide (PI), also showed a similar dose-dependent increase (FIG. 9B). The inventors then examined whether the AAV ITRs were sufficient to induce cell death as previously reported in embryonic stem cells (13). NPCs were electroporated with “high” (5 E6 copies/cell) and “low” (1 E6 copies/cell) doses of 145 bp AAV2 ITR ssDNA, scrambled ITR sequence, or water and plated for imaging as above or for FACS analysis (FIG. 10A-E). In the high-dose ITR condition, NPCs were significantly decreased by 6 hours post-electroporation (6-hour ITR 5 E6 vs H20: −8.1%±2.5, p<0.05) and had ceased expansion by 40 hours. Low-dose ITR and low-dose scramble groups were indistinguishable from H₂O. A transient decrease in the high-dose scrambled condition relative to H₂O was observed, but was minimal compared to high-dose ITR (FIG. 11). The proportion of dying PI+ cells was substantially greater in the high ITR condition relative to H₂O. Both low- and high-dose scrambled groups had a slight increase in cell death relative to H₂O that was minimal compared to the effect of high-dose ITR. FACS analysis at 12 and 24 hours showed the proportion of cells in S/G2 phase that were dying (UVZombie+) was greatly increased in the high ITR condition, but not in the other experimental groups relative to H₂O control (12 h ITR 5E6 vs H20+12.0%±2.0%, p<0.001, FIG. 10D). The proportion of non-replicating cells that were dying was <1% in all groups (FIG. 10E). Taken together with the empty capsid experiments (FIG. 10E), these experiments provide strong evidence that the AAV ITRs are sufficient and necessary to kill proliferating NPCs.

Experimental Design and Methods:

Perform pull-down experiments in primary mouse NPCs and human IPS-derived NPCs from AD patients and aged matched controls to determine if tau binds the rAAV genome. Mouse hippocampal NPCs: NPCs will be harvested from embryonic C57BL/6 mouse hippocampi and cultured onto polyornithine/laminin-coated (Sigma) plastic plates, grown in NPC media containing Dulbecco's modified Eagle's medium (DMEM)/F-12 (Invitrogen) supplemented with N2 and B27 (N2B27 medium, Invitrogen) in the presence of FGF2 (20 ng/ml), EGF (20 ng/ml), laminin (1 μg/ml), and heparin (5 μg/ml) and passaged with Accutase (Chemicon) as described previously (122, 123).

Human hippocampus patterned NPCs (hpNPCs): Human IPS cells derived from fibroblasts from sporadic AD patients and age-matched subjects (UCSD ADRC, courtesy of Gage Lab) will be cultured on Cultrex Matrix (Trevigen) coated 6-well plates under feeder-free conditions in mTeSR1 medium (Stem Cells Technologies) and passed using Collagenase IV (Life Science, 1 mg/ml) as described previously (124). For generation of hpNPCs, IPS cells will be plated onto low-adherence dishes without FGF2 in mTeSR1 medium with Rock inhibitor (Enzo Life Sciences, 10 μM) to generate floating embryoid bodies (EBs). EBs are treated with DKK1 (0.5 mg/ml), SB431542 (10 mM), Noggin (0.5 mg/ml) and cyclopamine (1 mM) in DMEM)/F12 plus N2 and B27 supplements (Invitrogen) as described previously (125, 126). EBs are treated for 20 days and then plated onto polyornithine/laminin (Sigma)-coated dishes in DMEM/F12 plus N2B27 medium and laminin (1 μg/ml) to facilitate attachment. Within days, rosettes are manually collected and dissociated with Accutase and plated onto polyornithine/laminin-coated dishes with NPC media (DMEM/F12, N2, B27, and 20 ng/ml FGF2).

Nuclear Pull-down Assay: The ITR pull-down assay was developed in the Shtrahman lab, adapted from (127) with guidance from the Rosenfeld lab at UCSD (128-130) and described below: 1) Isolation of nuclei: 2 E7 NPCs are washed 3× with ice cold PBS and then swelled in 10 ml of swelling buffer (10 mM Tris-HCl pH7.5, 2 mM MgCl2, 3 mM CaCl2) for 5 min on ice, harvested with a cell scraper, and centrifuged at 400 g for 10 min. Cells are then resuspended in 1 ml of lysis buffer (swelling buffer plus 10% glycerol), vortexed gently, and placed on ice for 5 min. Additional lysis buffer is added to achieve a total of 10 mL and centrifuged at 600 g for 6 min. The supernatant may be saved for cytosolic pull-down assays, if needed. The resulting nuclei are washed with 10 ml lysis buffer and centrifuged 2×. 2) Nuclear Protein Extract: Pellet is lysed with 200 μL of NP40 lysis buffer (50 mM Tris pH 8.0, 150 mM NaCl, 1% Nonidet P-40, protease inhibitor freshly added). Sonication (Bioruptor, Diagenode) is performed 2× on ice before rotating at 4 C for 20 min, and centrifuging at >15,000 g at 4 C for 15-30 min before supernatant is transferred to a fresh tube. 3) Pre-clear Nuclear Proteins: 60 μL of Dynabeads M-280 Streptavidin (Thermo Fisher) are separated or “pulled down” from their storage buffer with a magnet and washed 2× in 500 μL of 1:1 solution of PBS and lysis buffer. The washed beads are added to the nuclear protein extract with a final volume 500 μL and incubated at 4 C with rotation for 30 min. The mixture is pulled down and supernatant is collected. 25 μL of the pre-cleared extract it set aside for “input control” for western analysis.

Pull-Down Nuclear Proteins with Biotinylated ITRs: Biotinylated ITR or scrambled ITR DNA is dissolved in deionized H20 at 1 ug/ul. 10 μL are aliquoted into a PCR tube and heated to 95 C for 1 h, and allowed to cool to room temperature. DNA is added to 500 μg of pre-cleared extract and rotated for 1 hour at room temperature, allowing proteins to bind DNA. 60 μL of washed streptavidin beads and lysis buffer are added to reach 600 μL and rotated for 2 h. Mixture is magnetically separated and supernatant is removed. Beads are washed 3× for 8 min with rotation in 1 mL wash buffer (PBS plus proteinase and phosphatase inhibitor) and then supernatant is discarded. Protein loading buffer is added to the beads and protein mixture and heated to 95 C for 10 min, denaturing proteins. The protein mixture with beads is magnetically separated, the supernatant is collected, and protein concentration is determined by the Bradford assay (Bio-Rad). Supernatant is run on a western blot for total tau (tau-5, AHB0042, Life Technologies) and phosphorylated tau (AT8 against S202 and T205 phosphorylation sites, MN1020, Thermo Scientific). Visualization using enhanced chemiluminescence (Pierce) will be performed and quantified by densitometry (131-133).

Perform ImmunoFISH experiments in mouse and human NPCs in vitro and mouse NPCs in vivo to confirm whether tau colocalizes with rAAV genome. ImmunoFISH: The immunoFISH assay was adapted with guidance from Rosenfeld lab (134) and will be used to confirm the colocalization of tau with the rAAV ITR in the cell. NPCs (10K/well) will be plated on 16-micro-well plates with removable coverglass (CultureWell Grace Bio-Labs) and coated with polyornithine/laminin. NPC will be infected with AAV-CMV.EGFP (Addgene 105530-AAV1) at a range of MOIs similar to experiments described in FIG. 9. A 30 bp DNA probe (Integrated DNA Technologies) antisense to the coding strand of

GFP (5′-CTTGAAGAAGTCGTGCTGCTTCATGTGGTC-3′-Atto-565) (SEQ ID NO: 1), conjugated to Atto-565, will be used to localize the rAAV genome within the nucleus. A validated random negative control DNA probe of identical length will also be synthesized. NPCs will be fixed 12 h and 18 h post-infection with 4% paraformaldehyde in PBS for 8 min and then quenched with 0.1 M Tris-HCl (pH 7.4) for 5 min. Cells are washed with PBS and stored at 4° C. until used. Before hybridization, cells are washed with 2× saline sodium citrate (SSC) buffer (BioPioneer) for 3 min on shaker, incubated in 0.1 M HCl for 10 min, at washed with PBS 3×. Cells are permeabilized in 0.5% TX-100 in 1×PBS for 30 min and washed in PBS 3×. Cells are then incubated in 5% BSA in PBS containing 100 μg/L RNAse A for 1 h at 37° C., followed by equilibration in 50% formamide and 2×SSC for 1 h. The coverglass is removed prior to hybridization. 125 ng (1 μL) of probe plus 4 μL of 2× hybridization buffer (4×SSC w/ 40% dextran sulfate) is added to glass slide for each well, and the coverslip is placed cell-surface down onto the slide such that probe is contacting each well. Slides are heated for 7 min on a hotplate 80° C., allowed to cool gradually to 37 C, and placed in a humidified dark chamber at 37 C for 18-24 h. Next, each coverslip is washed 3× in 50% formamide and 2×SSC for 10 min and then in 2×SSC for 5 min 2× at 37 C. Cells are incubated first with PBS containing 0.1% Triton X-100 (PBST) and 5% BSA for 5 min and then primary antibodies for tau (˜1:100 in 2.5% BSA in PBST) are added for 1 h at 37° C. Cells are washed 3× in PBST for 8 min and incubated with fluorescent conjugated secondary antibody (˜1:500 dilution in 2.5% BSA in PBST) for 30 min at 37 C and washed again 2× in PBST for 8 min and 1 time in PBS for 5 min. Finally, cells are rinsed in distilled H₂O and mounted with DAPI.

A similar protocol will be adapted for immunoFISH in hippocampal brain slices obtained from animals sacrificed 12-18 hours after injection with AAV-CMV.EGFP into the DG. Extra procedures are often required for tissue pretreatment to increase permeability of FISH probes. In some cases, standard proteinase (PK, Roche Diagnostic) can be used without degrading protein epitopes for immunolabeling (95, 135). In other cases, pretreatment with 2×SCC is adequate without proteinase treatment to obtain adequate probe penetration and hybridization. For refractory cases, the inventors will perform antigen retrieval via tissue heating (135-137). These factors will be optimization for each pair of tau antibody and FISH probe to be tested.

Determine whether tau KO rescues rAAV-induced ablation of neurogenesis. Similar to experiments described in FIG. 7A-G, tau ko (JAX: 007251) and littermate controls will be given BrdU for 3 days and then 1 week later injected unilaterally with 3 E12 gc/mL AAV1-CAG-flex-eGFP and sacrificed 2 days or 1-week post injection. These pre-injection and post-injection time points are chosen due their incomplete loss of BrdU and neurogenesis markers (FIG. 7A-G, 8A-G) providing adequate dynamic range to observe either accentuation or rescue of rAAV-induced toxicity. The loss of BrdU+, Tbr2+, and DCX+ cells, relative to the contralateral HC, will be compared between tau ko and control mice.

Anticipated results, potential pitfalls, and alternative approaches: The inventors predict that affinity assays probing for ITR-binding proteins will isolate tau to a greater extent than assays using scrambled ITR DNA. If tau truly binds ITR DNA within the cell, then FISH experiments should confirm that tau and the rAAV genome colocalize within NPCs in culture and in the SGZ in vivo. The inventors also predict that injecting rAAV into the DG of tau ko mice will result in greater survival of NPCs compared to wildtype mice.

If the predictions above are true, future pull-down experiments testing ITRs with a mutated tau binding motif will be performed to verify that this site in required for binding. Although the AAV ITR is highly conserved, it may be possible to mutate the tau binding region without effecting the packaging and expression of rAAV. This engineered AAV will have significant implications for gene therapies for AD (138) and other CNS diseases, where the use of standard rAAV could ablate adult neurogenesis and potentially exacerbate AD or counteract treatments that promote neurogenesis such as memantine (37).

While the proposed experiments are focused on tau, studies also indicate that Aβ also binds nuclear DNA and is involved in the detection of foreign microbes, including viruses (67, 69, 139). High quality antibodies exist for Aβ, and knockout mice lacking APP or BACE1 are commercially available. Therefore, probing Aβ's binding affinity to AAV ITR and its role in AAV-induced ablation of neurogenesis using the strategies outlined above would be straight forward and practical. Finally, depending on the outcome of these targeted studies, the affinity pull-down experiments can be coupled with mass spectrometry to search of ITR-binding proteins and their downstream pathways in an unbiased fashion that may lead to novel therapeutic targets for AD.

Determine whether rAAV infection results in the production and spread of pathological tau species that contribute to the pathogenesis of AD. Hypothesis: rAAV infection will induce pathological tau and other signs of tau-related toxicity. Rationale: A number of environmental stressors in neurons, including HSV infection, trigger the formation of hyper-phosphorylated tau, a pathological tau species that aggregates and is the major component of neurofibrillary tangles in AD. In this example, the inventors will determine if rAAV infection can induce the formation and spread of pathological tau species in human IPS-cell derived and mouse models of AD. The inventors will also quantify the density of pre- and post-synaptic proteins as markers of synaptic dysfunction and toxicity. The inventors hypothesize that rAAV infection will: 1) induce the production hyper-phosphorylated tau in neurons 2) upon seeding with tau aggregates will lead to enhanced tau deposition and spreading to adjacent brain regions compared to seeding alone, and 3) will induce synaptic toxicity and decrease the number of synaptic contacts. The inventors predict that these virus-induced changes will be more prominent in AD models compared to controls.

Preliminary Data: In order to investigate the effect of rAAV on the production of hyperphosphorylated tau, the inventors injected n=3 mice with AAV1-CAG-flex-eGFPin to the DG and sacrificed animals and performed IHC for p-tau (AT8) 4 weeks post injection. All animals exhibited marked increase in p-tau in the injected compared to uninfected HC, extending beyond the DG (FIG. 11). Thus, AAV induces pathological tau species in wildtype mice.

Experimental Design and Methods:

Determine if rAAV infection induces production and spread of pathological tau species in HC neurons from 5×FAD and wildtype mice in vitro Primary neuronal culture: Primary neurons from the cortices of postnatal day 0-1 5×FAD mice on C57/Bl6 background from MMRRC (34848-JAX) and wild type littermate mouse pups will be cultured onto poly-ornithine-coated glass coverslips or glass bottom micro-well-plates using established protocols in the Chen lab (131, 132).

After 11-14 days in vitro (div), high titer AAV will be added at varying MOIs from 0 to 1 E 7 similar to FIG. 8. Cells will be fixed in paraformaldehyde as above and immunocytochemistry (ICC) will be performed to quantify total tau (tau-5), phosphorylated tau (AT8, courtesy Chen lab), and density of synaptic markers (PSD95 and synaptophysin, Abcam) at 2 days, 1 week, and 1 month post infection. For western analysis, cells will be homogenized at identical time points as ICC experiments above in radioimmunoprecipitation assay (RIPA) buffer (Sigma) containing protease inhibitor cocktail (Sigma), 1 mM phenylmethyl sulfonyl fluoride, phosphatase inhibitor cocktail (Sigma), 5 mM nicotinamide (Sigma), and 1 μM trichostatin A (Sigma). After sonication, lysates are centrifuged at 170,000 g at 4° C. for 15 min and 18,000 g at 4° C. for 15 min. Supernatants will be collected, quantified, and undergo western analysis and quantified by densitometry for total tau (tau-5), phosphorylated tau (AT8), and total synaptic markers (PSD95 and synaptophysin). In separate experiments, neurons from 5×FAD and wildtype mouse pups will be cultured onto 96-well plates and virus will be added as above at 11-14 days dvi. Cell death and viability will be monitored by time lapse imaging analogous to experiments in FIG. 8A-G (10).

For experiments investigating the effect of AAV on fibril-induced tau spreading (131, 132), neurons are plated in a custom (124, 140) or commercially available (XonaChip) microfluidic culture chamber system. These chambers contain a microgroove that allow neuronal processes extending from neurons plated in each chamber to crossover and make synapses with neurons in the adjacent chamber. Using unequal volumes, a pressure gradient is established in the microfluidic culture plate such that molecules and virus can only flow gradually across the microgroove in one direction, such that virus added to the downstream chamber will not enter the upstream chamber. Chambers are coated with 0.5 mg/ml poly-L-lysine (PLL, MW 70,000-150,000, Sigma) overnight, washed three times with DI water, and air-dried. Devices are coated with polyornithine/laminin before plating. For these experiments, rAAV expressing GFP is added to the downstream chamber after 3 div, before neuronal processes cross microchamber, for 24 hours, infecting only neurons in this chamber. The following day, tau fibrils purified from AD brains ((141), courtesy Chen lab) will be added to the downstream well at 100 nM concentration and replenished every other day for total of 7 days. Cells will be fixed at 1-week and 1-month post infection. Control experiments using only AAV or only tau seeds (fibrils) will also be performed. ICC in the upstream “receptor” chamber will be performed quantifying the spread of total tau, phosphorylated tau, and tau aggregates (MC1, aggregated conformation-specific tau, courtesy of Dr. Peter Davies). In addition, GFP expression in neuronal cell bodies in the upstream well will be used to rule out virus infected neurons in this well, which would complicate interpretation of the effect of virus on tau spread.

Determine if rAAV infection induces production of pathological tau species in HC neurons from AD patients and controls. To obtain mature neurons, the inventors will utilize a protocol that is enriched (32%, Yu et al) for Prox1+ DGCs. As previously described (124-126), hpNPCs from AD and aged matched control patients in previous experiments will be plated on a monolayer of hippocampal astrocytes in the presence of ascorbic acid (Sigma, 200 nM), cAMP (Fisher Scientific, 500 mg/ml), BDNF (20 ng/ml), laminin (1 μg/ml), Wnt3a (R&D Systems, 20 ng/ml), and 1% fetal bovine serum. Wnt3a will be removed after 3 weeks and neurons will mature for at least 3 months before testing.

Assays for quantification of total tau, production of pathological tau species, and changes in synaptic density in response to AAV infection will be performed in a similar fashion as described above. In addition, for IPS-derived neurons the inventors will take advantage of a number of human specific antibodies including those against alternative phosphorylation sites on tau (PHF1 (S396/404), S262) and other forms of pathological tau including acetylated tau (K174, K274 courtesy of Chen lab, (131, 132)). The remaining antibodies described above will also be effective for human tau and its variants.

Determine if rAAV infection induces production and spread of pathological tau species in the HC of 5×FAD and wildtype mice in vivo. Experiments will be performed on 6-month 5×FAD mice (MMRRC, 34848-JAX) and wildtype littermate controls. Mice will be injected with 3E12 AAV-CAG-eGFP or saline unilaterally into the DG at 6 months of age and sacrificed at 2 days, 1 week, and 1 month post infection similar to experiments in FIG. 5. IHC and for total tau (tau-5), p-Tau (AT8), and synaptic density (PSD95, synaptophysin) will be performed and quantified in the ipsilateral and contralateral dentate gyri. Similar viral injections and time points will be performed for western analysis in bilateral HC using similar extraction protocols as for cells described above (131, 132).

Protocols developed in the Chen lab will be use to investigate the effects of AAV fibril-induced tau spreading (131, 132), mice receive injections of saline, 3E12 AAV, AD tau seeds, or both AAV plus seed unilaterally into the DG as above. Mice will be sacrificed at 1 month and 3 months post infection. Again, IHC will be performed for and for total tau (tau-5), p-Tau (AT8), tau aggregates (MC1), and synaptic density (PSD95, synaptophysin) with quantification in each hippocampal subfield (DG, CA3, CA1) and the EC bilaterally. Western analysis quantifying these proteins in HC and EC bilaterally will be performed as described above.

Anticipated results, potential pitfalls, and alternative approaches: The inventors anticipate that rAAV infection in vitro and in vivo will lead to increased deposition of tau and its pathological variants, including hyper-phosphorylated tau. The inventors expect this increase to be greater in IPS-derived neurons from AD patients and in the neurons and HC of 5×FAD mice than respective controls. The inventors also predict that rAAV infection will enhance any synaptic toxicity observed in these AD models, manifested by fewer number of postsynaptic density protein 95 (PSD95)+ and synaptophysin+ puncta and less total synaptic proteins. While tau seeds have been shown to induce the formation or spread of tangles, even in models lacking tau mutations (141), it is possible that the inventors will only observe this in the setting of rAAV toxicity, particularly in the AD mouse models. However, this process may require repetitive seeding or long incubation periods, which may not be experimentally tractable. Regardless, the inventors do expect that experiments delivering both rAAV and tau seeds are likely to produce increased tau deposition in infected neurons and their uninfected synaptic partners, compared to rAAV or seeds alone.

There are also a number of other pathological tau species that can be tracked in future studies including cleaved tau products (142). Further expanding on the alternatives studies described earlier, it would be interesting and practical to explore the possibility that AAV infection induces increased secretion of Aβ in the various experimental contexts, including earlier deposition of amyloid in the 5×FAD mouse model of AD. Finally, the inventors chose IPS-derived hippocampal neurons (124, 126) to model the role of AAV and tau in human models of sporadic AD. The Gage lab has demonstrated that induced neurons (IN), made from direct conversion of patient fibroblasts, retain many of the epigenetic signatures of aging in patient derived lines (143), and in principle may be preferable for modeling the combined effect of aging, viral infection, and other risk factors for AD. Unfortunately, the conversion is performed through use of a lentiviral vector, which also attenuates adult neurogenesis in the mouse (unpublished, data not shown) and may select for cells that have some level of resistance to viral toxicity. However, this may be a viable option in future studies.

Finally, it is possible the rAAV infection is not sufficient to cause increased production and spread of pathological protein species and that expression of the Rep protein by wildtype AAV, which also has toxic effects on the cell (144), is required. If infection with rAAV fails to induce increased production of pathological tau species or other pathological phenotypes, the inventors will encode the rep gene with rAAV to model the effects of latent infection by wildtype AAV. Full wildtype AAV can also be produced with the addition of the cap gene if necessary.

Scientific Rigor

1) based on preliminary ViroFind studies, a power analysis (β=0.2, α=0.05), yielded 35 AD samples and 17 controls (2:1 allocation) to detect a 30% difference incidence of AAV between AD and control. This sample size is similar to that in previous reports examining the loss neurogenesis in AD patients (4). AD will be designated as the primary outcome. The primary exposure will be the presence of virus and density of neurogenesis markers. Potential confounders consisting of key risk factors for AD, such as ApoE genotype, will be included in the data set. Univariable analysis will be conducted using χ2 tests for categorical variables and two-sample t-tests for continuous variables. Continuous variables will be converted to categorical variables using relevant cutoffs. Multinomial categorized dependent logistic regression modeling will be used as a dependent variable has >1 category (Braak stages). It will include all variables that differ between AD and controls at a significance level of 0.1 to estimate the independent contribution of each risk factor. Statistical analyses of regression models, performed using SPSS (IBM), will be considered significant at the level of 0.05.

For in vivo immunoFISH experiments, the inventors estimate sample size based on preliminary data for experiments using histological markers measured at 12 h and 18 h prior to the elimination of dividing cells (FIG. 8A-G). The inventors estimate n=7/group per time point (β=0.2, α=0.05), based on a coefficient of variation of ˜40% and the ability to detect a 2.5-fold increase in marker-genome colocalization. The above 12 h/18 h experiments are generally the most variable and are used conservatively to estimate sample size for rAAV mouse experiments. However, these calculations have considerable uncertainty and the inventors have increased the estimate for all experiments to 10 mice per group for each gender, condition, and time point. 2) One-way and 2-way ANOVAs adjusted for multiple comparisons will be used to compare key dependent variables such as the density of neurogenesis markers in the different experimental conditions and time points. All analyses will be performed with SPSS. 3) The inventors will use appropriate controls including scrambled ITR DNA and wild-type littermates whenever possible. 4) Data analysis will be performed by blinded investigators. 5) Human studies will be sex-matched.

Male and female mice will both be used and first be analyzed separately, then combined if results are similar.

### Example 5

Parkinson's disease (PD) is a neurodegenerative brain disease affecting 1 million people in the US. Available treatments are symptomatic but cannot stop or modify the course of the disease (1). The clinical and pathological characteristics of PD are well known (2). The scientific question the inventors want to answer is what triggers PD.

Numerous studies have implicated viruses, as causal factors or potential triggers for PD, since a Parkinsonian epidemic ensued in survivors of the 1918 encephalitis lethargica (3-16). Indeed, viruses may remain latent in the CNS, and reactivate during normal aging. Recurrent neuronal damage caused directly by viral infection and indirectly by virus-induced inflammation may lead ultimately to PD. In fact, the induction of α-synuclein may be secondary, or possibly even a defense mechanism against viral infection (4, 6, 10, 13).

In addition, genetic factors have been implicated in the pathogenesis of PD. Rare mutations in several genes cause familial PD, accounting for <10% of all PD cases (17). In addition, 90 independent common variants significantly increase PD risk, especially in combination (18). The role of variants that modify PD pathogenesis in mutation carriers is beginning to emerge (19-21). However, genetic factors only accounts for ˜30% of the heritability (18) indicating that novel genetic associations remain to be discovered, especially interactions of germline variation with viruses.

The rationale for the collaborative approach is to combine the expertise of Neuro-Virologists and Neuro-Geneticists to decipher for the first time the interplay of viruses and genetic factors in PD pathogenesis. This has never happened before, since virologists and PD experts do not usually interact and because of lack of dedicated funding for such project. The Koralnik and Lubbe labs are poised to join forces and break down those old silos.

Pilot Projects Goals

During the pilot period, the inventors will: Characterize the entire virome in PD patients and control subjects in the US and Zambia using ViroFind, and analyze their genetic markers. The inventors have developed a novel deep sequencing-based platform for detection of all viruses know to infect humans, the entire “virome” in clinical samples. This assay, named “ViroFind” can detect 561 viral species, and potentially, novel viruses (22). An example ViroFind workflow is shown in FIG. 1. The inventors will identify viruses in a total of 60 subjects: fresh frozen post-mortem brain samples at 3 locations classically affected in PD brains in 10 PD patients, 10 degenerative controls with multiple system atrophy (MSA) and 10 with progressive supranuclear palsy (PSP), and 10 age-matched subjects without degenerative brain diseases. Samples will be collected from the brain banks from the Rush Alzheimer's Disease Center and from the Mayo Clinic, Jacksonville, Fla. The inventors will also characterize the virome in the blood and/or CSF of 10 live Zambian patients with PD and 10 Zambian controls at the Global Neurology program in Lusaka, Zambia.

In addition, the inventors will define the contribution of the 90 known common variants associated with PD in these 60 samples by genotyping all European-ancestry samples (cases and controls) on the NeuroChip array and the Zambian samples on the H₃Africa array using GenomeStudio (Ilumina). Visual confirmation of a subset will be performed to assess the accuracy of the genotyping. Standardized individual and variant level quality controls will be performed. The inventors will then extend the correlation of host genetic variants with the observed viruses and/or virus variants to search for novel interactions that influence PD risk.

Search the Parkinson's Progression Markers Initiative (PPMI) genomic database for viruses and genetic variants. The inventors will search the PPMI database containing DNA/RNA from 100 PD patients and 100 controls for viral sequences using ViroFind as well as a genomic variants pipeline. Following standardized data processing and quality control (GATK Best Practices, http://www.broadinstitute.org/), the inventors will first characterize the contribution of 90 known PD risk variants in the current samples, and examine whether or not these variants are correlated with any virus or variant observed in a virus identified in modulating PD risk.

The inventors will then expand this to look at all variants interrogated to examine novel genetic interactions between host genetics and viruses and viral variation.

This pilot study will lay the foundation for the longer-term project, where the inventors will expand studies in larger populations of US and Zambian patients. The inventors will also study the location of the viruses in the brain by immunohistochemistry in contralateral fixed brain samples from brain bank cases, study the immune response to those viruses and devise therapeutic or preventive interventions in PD patients.

Tools and Resources

The inventors will bring the ViroFind and the Genomic variant pipelines to this pilot project. Resources include Northwestern University computational cluster—a dedicated 800-node high-performance computing system named Quest, and its 102-node Genomics Computer Cluster. The inventors hope to automatize these pipelines and transform them into a clinically actionable tools that can be used for the management of PD patients in real time. The inventors could also devise kits for molecular identification of relevant viruses that could be used in resource-limited setting as well. If novel viruses are discovered that are associated with PD, the inventors will further define their life cycle in vitro and in animal studies.

The inventors hope to collaborate and share these tools and resources with others in the Challenge Network, to examine the interplay of viral and genetic factors in other degenerative diseases such as Alzheimer's or Amyotrophic Lateral Sclerosis.

The inventors hope to benefit from the expertise from the Challenge Network and CZI in the development of the new field of “Viromics”. This will facilitate a systems biology approach, integrating viral strains, genetic variants, cellular targets, transcriptional activity, metabolic patterns and immunological responses in a holistic manner, and to create a publicly available Viromics database. The ViromicsDB will integrates virological data together with genomics, transcriptomics, metabolomics, immunomics and pathobiology in the human host, with the goal of defining druggable targets. The inventors could also benefit from Facebook to connect with PD patients worldwide and gather population and epidemiological data on PD patients, exposure to viruses and effects of treatment.

### REFERENCES

1. Olanow C W, Kieburtz K. 2010. Defining disease-modifying therapies for PD—a road map for moving forward. Mov Disord 25: 1774-9.
2. Braak H, Rub U, Gai W P, Del Tredici K. 2003. Idiopathic Parkinson's disease: possible routes by which vulnerable neuronal types may be subject to neuroinvasion by an unknown pathogen. J Neural Transm (Vienna) 110: 517-36.
3. Vilensky J A, Goetz C G, Gilman S. 2006. Movement disorders associated with encephalitis lethargica: a video compilation. Mov Disord 21: 1-8.
4. Jang H, Boltz D A, Webster R G, Smeyne R J. 2009. Viral parkinsonism. Biochim Biophys Acta 1792: 714-21.
5. Caggiu E, Paulus K, Galleri G, Arm G, Manetti R, Sechi G P, Sechi L A. 2017. Homologous HSV1 and alpha-synuclein peptides stimulate a T cell response in Parkinson's disease. J Neuroimmunol 310: 26-31.
6. Mori I. 2017. Viremic attack explains the dual-hit theory of Parkinson's disease. Med Hypotheses 101: 33-6.
7. Massey A R, Beckham J D. 2016. Alpha-Synuclein, a Novel Viral Restriction Factor Hiding in Plain Sight. DNA Cell Biol 35: 643-5.
8. Lam M M, Mapletoft J P, Miller M S. 2016. Abnormal regulation of the antiviral response in neurological/neurodegenerative diseases. Cytokine 88: 251-8.
9. Lai S W, Lin C H, Lin H F, Lin C L, Lin C C, Liao K F. 2017. Herpes zoster correlates with increased risk of Parkinson's disease in older people: A population-based cohort study in Taiwan. Medicine (Baltimore) 96: e6075.
10. Beatman E L, Massey A, Shives K D, Burrack K S, Chamanian M, Morrison T E, Beckham J D. 2015. Alpha-Synuclein Expression Restricts RNA Viral Infections in the Brain. J Virol 90: 2767-82.
11. Limongi D, Baldelli S. 2016. Redox Imbalance and Viral Infections in Neurodegenerative Diseases. Oxid Med Cell Longev 2016: 6547248.
12. Chen H H, Liu P F, Tsai H H, Yen R F, Liou H H. 2016. Re: Wangensteen et al. of a letter on ‘Hepatitis C virus infection: a risk factor for Parkinson's disease.’. J Viral Hepat 23: 560.
13. Goldeck D, Maetzler W, Berg D, Oettinger L, Pawelec G. 2016. Altered dendritic cell subset distribution in patients with Parkinson's disease: Impact of CMV serostatus. J Neuroimmunol 290: 60-5.
14. Lutters B, Foley P, Koehler P J. 2018. The centennial lesson of encephalitis lethargica. Neurology 90: 563-7.
15. Brunetti V, Testani E, Iorio R, Frisullo G, Luigetti M, Di Giuda D, Marca G D. 2016. Post-Encephalitic Parkinsonism and Sleep Disorder Responsive to Immunological Treatment: A Case Report. Clin EEG Neurosci 47: 324-9.
16. Dourmashkin R R, Dunn G, Castano V, McCall S A. 2012. Evidence for an enterovirus as the cause of encephalitis lethargica. BMC Infect Dis 12: 136.
17. Lubbe S, Morris H R. 2014. Recent advances in Parkinson's disease genetics. J Neurol 261: 259-66.
18. Lubbe S J, Escott-Price V, Brice A, Gasser T, Hardy J, Heutink P, Sharma M, Wood N W, Nalls M, Singleton A B, Williams N M, Morris H R, International Parkinson's Disease Genomics C. 2016. Is the MC1R variant p.R160W associated with Parkinson's? Ann Neurol 79: 159-61.
19. Lubbe S J, Escott-Price V, Gibbs J R, Nalls M A, Bras J, Price T R, Nicolas A, Jansen I E, Mok K Y, Pittman A M, Tomkins J E, Lewis P A, Noyce A J, Lesage S, Sharma M, Schiff E R, Levine A P, Brice A, Gasser T, Hardy J, Heutink P, Wood N W, Singleton A B, Williams N M, Morris H R, for International Parkinson's Disease Genomics C. 2016. Additional rare variant analysis in Parkinson's disease cases with and without known pathogenic mutations: evidence for oligogenic inheritance. Hum Mol Genet 25: 5483-9.
20. Escott-Price V, International Parkinson's Disease Genomics C, Nalls M A, Morris H R, Lubbe S, Brice A, Gasser T, Heutink P, Wood N W, Hardy J, Singleton A B, Williams N M, members Ic. 2015. Polygenic risk of Parkinson disease is correlated with disease age at onset. Ann Neurol 77: 582-91.
21. Jansen I E, Gibbs J R, Nalls M A, Price T R, Lubbe S, van Rooij J, Uitterlinden A G, Kraaij R, Williams N M, Brice A, Hardy J, Wood N W, Morris H R, Gasser T, Singleton A B, Heutink P, Sharma M, International Parkinson's Disease Genomics C. 2017. Establishing the role of rare coding variants in known Parkinson's disease risk loci. Neurobiol Aging 59: 220 e11-e18.
22. Chalkias S, Gorham J M, Mazaika E, Parfenov M, Dang X, DePalma S, McKean D, Seidman C E, Seidman J G, Koralnik I J. 2018. ViroFind: A novel target-enrichment deep-sequencing platform reveals a complex JC virus population in the brain of PML patients. PLoS One 13: e0186945.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. Any cited references are incorporated by reference herein in their entireties. In the event that there is an inconsistency between a definition of a term in the specification as compared to a definition of the term in a cited reference, the term should be interpreted based on the definition in the specification.

TABLE 1
Probe coverage coordinates by NCBI genome ID.

| Probe Code | Start Position | End Position | Genome ID |
|---|---|---|---|
| chr2 | 0 | 1841 | NC_002208.1 |
| chr3 | 0 | 110160 | NC_001348.1 |
| chr3 | 110280 | 124884 | NC_001348.1 |
| chr4 | 0 | 7614 | NC_001354.1 |
| chr5 | 0 | 7902 | NC_001355.1 |
| chr6 | 0 | 5176 | NC_001358.1 |
| chr7 | 0 | 13246 | NC_001364.1 |
| chr8 | 0 | 35937 | NC_001405.1 |
| chr9 | 0 | 4200 | NC_001430.1 |
| chr9 | 4320 | 6480 | NC_001430.1 |
| chr9 | 6600 | 7080 | NC_001430.1 |
| chr9 | 7200 | 7390 | NC_001430.1 |
| chr10 | 0 | 7176 | NC_001434.1 |
| chr11 | 0 | 8507 | NC_001436.1 |
| chr12 | 0 | 10976 | NC_001437.1 |
| chr13 | 0 | 4697 | NC_001442.1 |
| chr14 | 0 | 11444 | NC_001449.1 |
| chr15 | 0 | 34214 | NC_001454.1 |
| chr16 | 0 | 7353 | NC_001457.1 |
| chr17 | 0 | 7348 | NC_001458.1 |
| chr18 | 0 | 34125 | NC_001460.1 |
| chr19 | 0 | 9189 | NC_001463.1 |
| chr20 | 0 | 7389 | NC_001472.1 |
| chr21 | 0 | 10735 | NC_001477.1 |
| chr22 | 0 | 7835 | NC_001479.1 |
| chr23 | 0 | 8952 | NC_001488.1 |
| chr24 | 0 | 7478 | NC_001489.1 |
| chr25 | 0 | 7212 | NC_001490.1 |
| chr26 | 0 | 15894 | NC_001498.1 |
| chr27 | 0 | 11835 | NC_001512.1 |
| chr28 | 0 | 7746 | NC_001531.1 |
| chr29 | 0 | 5153 | NC_001538.1 |
| chr30 | 0 | 11932 | NC_001542.1 |
| chr31 | 0 | 9623 | NC_001549.1 |
| chr32 | 0 | 8280 | NC_001550.1 |
| chr32 | 8400 | 8557 | NC_001550.1 |
| chr33 | 0 | 15384 | NC_001552.1 |
| chr34 | 0 | 11161 | NC_001560.1 |
| chr35 | 0 | 10695 | NC_001564.1 |
| chr36 | 0 | 10862 | NC_002031.1 |
| chr37 | 0 | 7919 | NC_001576.1 |
| chr38 | 0 | 7855 | NC_001583.1 |
| chr39 | 0 | 7961 | NC_001586.1 |
| chr40 | 0 | 6960 | NC_001587.1 |
| chr40 | 7080 | 7723 | NC_001587.1 |
| chr41 | 0 | 7560 | NC_001591.1 |
| chr42 | 0 | 7856 | NC_001593.1 |
| chr43 | 0 | 8027 | NC_001595.1 |
| chr44 | 0 | 7434 | NC_001596.1 |
| chr45 | 0 | 8910 | NC_001607.1 |
| chr46 | 0 | 21720 | NC_001611.1 |
| chr46 | 21840 | 160800 | NC_001611.1 |
| chr46 | 160920 | 185578 | NC_001611.1 |
| chr47 | 0 | 7413 | NC_001612.1 |
| chr48 | 0 | 7152 | NC_001617.1 |
| chr49 | 0 | 5243 | NC_001669.1 |
| chr50 | 0 | 11141 | NC_001672.1 |
| chr51 | 0 | 7100 | NC_001690.1 |
| chr52 | 0 | 7184 | NC_001691.1 |
| chr53 | 0 | 7313 | NC_001693.1 |
| chr54 | 0 | 5130 | NC_001699.1 |
| chr55 | 0 | 9360 | NC_001710.1 |
| chr56 | 0 | 10359 | NC_001722.1 |
| chr57 | 0 | 4726 | NC_001729.1 |
| chr58 | 0 | 15225 | NC_001781.1 |
| chr59 | 0 | 1800 | NC_001786.1 |
| chr59 | 1920 | 8040 | NC_001786.1 |
| chr59 | 8160 | 11488 | NC_001786.1 |
| chr60 | 0 | 4680 | NC_001798.1 |
| chr60 | 4800 | 9000 | NC_001798.1 |
| chr60 | 9120 | 72120 | NC_001798.1 |
| chr60 | 72240 | 118080 | NC_001798.1 |
| chr60 | 118200 | 122520 | NC_001798.1 |
| chr60 | 122640 | 123600 | NC_001798.1 |
| chr60 | 123720 | 148200 | NC_001798.1 |
| chr60 | 148320 | 154746 | NC_001798.1 |
| chr61 | 0 | 9181 | NC_001802.1 |
| chr62 | 0 | 15191 | NC_001803.1 |
| chr63 | 0 | 120 | NC_001806.1 |
| chr63 | 360 | 960 | NC_001806.1 |
| chr63 | 1080 | 9000 | NC_001806.1 |
| chr63 | 9240 | 71640 | NC_001806.1 |
| chr63 | 71880 | 117120 | NC_001806.1 |
| chr63 | 117360 | 125280 | NC_001806.1 |
| chr63 | 125400 | 126120 | NC_001806.1 |
| chr63 | 126240 | 143760 | NC_001806.1 |
| chr63 | 143880 | 151920 | NC_001806.1 |
| chr63 | 152141 | 152261 | NC_001806.1 |
| chr64 | 0 | 10871 | NC_001809.1 |
| chr65 | 0 | 8040 | NC_001815.1 |
| chr65 | 8160 | 8855 | NC_001815.1 |
| chr66 | 0 | 4767 | NC_001829.1 |
| chr67 | 0 | 7348 | NC_001897.1 |
| chr68 | 0 | 8251 | NC_001918.1 |
| chr69 | 0 | 15690 | NC_001921.1 |
| chr71 | 0 | 6813 | NC_001943.1 |
| chr72 | 0 | 15140 | NC_001989.1 |
| chr73 | 0 | 4718 | NC_002077.1 |
| chr74 | 0 | 9028 | NC_000858.1 |
| chr75 | 360 | 8280 | NC_000898.1 |
| chr75 | 8760 | 129480 | NC_000898.1 |
| chr75 | 129720 | 133920 | NC_000898.1 |
| chr75 | 134160 | 153360 | NC_000898.1 |
| chr75 | 153720 | 161520 | NC_000898.1 |
| chr75 | 161994 | 162114 | NC_000898.1 |
| chr76 | 0 | 7320 | NC_000940.1 |
| chr77 | 0 | 11014 | NC_000943.1 |
| chr78 | 0 | 15456 | NC_002161.1 |
| chr79 | 0 | 2760 | NC_002195.1 |
| chr80 | 0 | 17904 | NC_002199.1 |
| chr81 | 0 | 3720 | NC_002470.1 |
| chr81 | 3840 | 4560 | NC_002470.1 |
| chr81 | 4680 | 6000 | NC_002470.1 |
| chr81 | 6120 | 6600 | NC_002470.1 |
| chr81 | 6720 | 6960 | NC_002470.1 |
| chr82 | 0 | 15384 | NC_002200.1 |
| chr83 | 120 | 480 | NC_001544.1 |
| chr83 | 600 | 720 | NC_001544.1 |
| chr83 | 840 | 2760 | NC_001544.1 |
| chr83 | 2880 | 3720 | NC_001544.1 |
| chr83 | 3840 | 7800 | NC_001544.1 |
| chr83 | 7920 | 8160 | NC_001544.1 |
| chr83 | 8280 | 8520 | NC_001544.1 |
| chr83 | 8640 | 8880 | NC_001544.1 |
| chr83 | 9000 | 9240 | NC_001544.1 |
| chr83 | 9480 | 9720 | NC_001544.1 |
| chr83 | 10200 | 10560 | NC_001544.1 |
| chr83 | 10680 | 10920 | NC_001544.1 |
| chr83 | 11160 | 11657 | NC_001544.1 |
| chr84 | 0 | 18959 | NC_002549.1 |
| chr85 | 0 | 8284 | NC_002551.1 |
| chr86 | 0 | 15462 | NC_001796.2 |
| chr87 | 0 | 10962 | NC_001563.2 |
| chr88 | 0 | 15186 | NC_002617.1 |
| chr89 | 0 | 10649 | NC_002640.1 |
| chr90 | 0 | 27317 | NC_002645.1 |
| chr91 | 0 | 7440 | NC_002058.3 |
| chr92 | 0 | 12301 | NC_002657.1 |
| chr93 | 0 | 1682 | NC_001653.2 |
| chr94 | 0 | 18246 | NC_002728.1 |
| chr95 | 0 | 16236 | NC_003043.1 |
| chr96 | 0 | 11822 | NC_003243.1 |
| chr97 | 0 | 4680 | NC_003310.1 |
| chr97 | 4920 | 152400 | NC_003310.1 |
| chr97 | 152640 | 169440 | NC_003310.1 |
| chr97 | 169560 | 178800 | NC_003310.1 |
| chr97 | 178920 | 192000 | NC_003310.1 |
| chr97 | 192120 | 196858 | NC_003310.1 |
| chr98 | 0 | 6240 | NC_003323.1 |
| chr98 | 6480 | 8918 | NC_003323.1 |
| chr99 | 0 | 146454 | NC_003389.1 |
| chr101 | 0 | 11411 | NC_003417.1 |
| chr102 | 0 | 15646 | NC_003443.1 |
| chr103 | 0 | 15600 | NC_003461.1 |
| chr104 | 0 | 10600 | NC_003635.1 |
| chr105 | 0 | 10116 | NC_003676.1 |
| chr106 | 0 | 10140 | NC_003675.1 |
| chr107 | 0 | 10839 | NC_003687.1 |
| chr108 | 0 | 10943 | NC_003690.1 |
| chr110 | 0 | 6927 | NC_003790.1 |
| chr111 | 0 | 11675 | NC_003899.1 |
| chr112 | 0 | 11484 | NC_003908.1 |
| chr113 | 0 | 3215 | NC_003977.1 |
| chr114 | 0 | 7055 | NC_003990.1 |
| chr115 | 0 | 10053 | NC_003996.1 |
| chr116 | 0 | 15522 | NC_004074.1 |
| chr117 | 0 | 9646 | NC_004102.1 |
| chr118 | 0 | 8033 | NC_004104.1 |
| chr120 | 0 | 10690 | NC_004119.1 |
| chr121 | 0 | 4888 | NC_004158.1 |
| chr122 | 0 | 18891 | NC_004161.1 |
| chr126 | 0 | 3376 | NC_004294.1 |
| chr127 | 0 | 3432 | NC_004293.1 |
| chr129 | 0 | 5028 | NC_004295.1 |
| chr131 | 0 | 10685 | NC_004355.1 |
| chr132 | 0 | 8126 | NC_004451.1 |
| chr133 | 0 | 9518 | NC_004455.1 |
| chr134 | 0 | 7461 | NC_004500.1 |
| chr135 | 0 | 11826 | NC_004162.2 |
| chr136 | 0 | 4754 | NC_001505.2 |
| chr137 | 0 | 3852 | NC_002076.2 |
| chr138 | 0 | 5135 | NC_004713.1 |
| chr139 | 0 | 29751 | NC_004718.3 |
| chr140 | 0 | 5256 | NC_004800.1 |
| chr142 | 0 | 15192 | NC_005036.1 |
| chr143 | 0 | 10857 | NC_005039.1 |
| chr144 | 0 | 10787 | NC_005062.1 |
| chr145 | 0 | 11375 | NC_005064.1 |
| chr146 | 0 | 3411 | NC_005081.1 |
| chr147 | 0 | 3439 | NC_005078.1 |
| chr148 | 0 | 3343 | NC_005077.1 |
| chr149 | 0 | 30738 | NC_005147.1 |
| chr151 | 0 | 3616 | NC_005219.1 |
| chr155 | 0 | 3635 | NC_005234.1 |
| chr156 | 0 | 3651 | NC_005237.1 |
| chr157 | 0 | 15702 | NC_005283.1 |
| chr159 | 0 | 16650 | NC_005339.1 |
| chr160 | 0 | 15378 | NC_005084.2 |
| chr162 | 0 | 240 | Y528864.1 |
| chr162 | 480 | 129480 | Y528864.1 |
| chr162 | 129600 | 130680 | Y528864.1 |
| chr162 | 131040 | 131217 | Y528864.1 |
| chr163 | 0 | 13335 | NC_004148.2 |
| chr164 | 0 | 7458 | NC_010624.1 |
| chr166 | 0 | 27553 | NC_005831.2 |
| chr167 | 0 | 7438 | NC_005134.2 |
| chr168 | 0 | 34246 | NC_006144.1 |
| chr169 | 0 | 4642 | NC_006152.1 |
| chr170 | 4080 | 9120 | NC_001716.2 |
| chr170 | 9960 | 143040 | NC_001716.2 |
| chr170 | 147120 | 152160 | NC_001716.2 |
| chr170 | 152520 | 152640 | NC_001716.2 |
| chr171 | 0 | 4721 | NC_006260.1 |
| chr172 | 0 | 4393 | NC_006261.1 |
| chr173 | 0 | 7429 | NC_006269.1 |
| chr174 | 0 | 4215 | NC_006320.1 |
| chr176 | 0 | 2265 | NC_006308.1 |
| chr177 | 0 | 3419 | NC_006447.1 |
| chr179 | 0 | 11940 | NC_006429.1 |
| chr180 | 0 | 18875 | NC_006432.1 |
| chr181 | 0 | 15450 | NC_006428.1 |
| chr182 | 0 | 15246 | NC_006430.1 |
| chr184 | 0 | 15882 | NC_006296.2 |
| chr185 | 0 | 11597 | NC_006558.1 |
| chr186 | 0 | 11066 | NC_006551.1 |
| chr187 | 0 | 1200 | NC_006554.1 |
| chr187 | 1320 | 7476 | NC_006554.1 |
| chr188 | 0 | 3402 | NC_006573.1 |
| chr189 | 0 | 3427 | NC_006575.1 |
| chr190 | 0 | 14885 | NC_006579.1 |
| chr191 | 0 | 34450 | NC_006879.1 |
| chr192 | 0 | 10653 | NC_006947.1 |
| chr193 | 0 | 194711 | NC_006998.1 |
| chr195 | 0 | 2249 | NC_007013.1 |
| chr196 | 0 | 2635 | NC_007014.1 |
| chr197 | 0 | 5268 | NC_007018.1 |
| chr198 | 0 | 15948 | NC_006383.2 |
| chr199 | 0 | 1565 | NC_007360.1 |
| chr202 | 0 | 18954 | NC_007454.1 |
| chr203 | 0 | 5299 | NC_007455.1 |
| chr206 | 0 | 10080 | NC_007605.1 |
| chr206 | 10200 | 36360 | NC_007605.1 |
| chr206 | 36480 | 37200 | NC_007605.1 |
| chr206 | 37320 | 95880 | NC_007605.1 |
| chr206 | 96600 | 171823 | NC_007605.1 |
| chr207 | 0 | 5230 | NC_007611.1 |
| chr208 | 0 | 15516 | NC_007620.1 |
| chr209 | 0 | 14071 | NC_007652.1 |
| chr210 | 0 | 29926 | NC_006577.2 |
| chr211 | 0 | 30480 | NC_007732.1 |
| chr213 | 0 | 19212 | NC_007803.1 |
| chr216 | 0 | 5079 | NC_007922.1 |
| chr217 | 0 | 5278 | NC_007923.1 |
| chr218 | 0 | 7654 | NC_001959.2 |
| chr219 | 0 | 7263 | NC_008188.1 |
| chr220 | 0 | 7259 | NC_008189.1 |
| chr221 | 0 | 4679 | NC_001401.2 |
| chr222 | 0 | 30307 | NC_008315.1 |
| chr223 | 0 | 10793 | NC_008719.1 |
| chr224 | 0 | 10510 | NC_008718.1 |
| chr225 | 0 | 84000 | NC_008724.1 |
| chr225 | 84120 | 115800 | NC_008724.1 |
| chr225 | 115920 | 252240 | NC_008724.1 |
| chr225 | 252360 | 254400 | NC_008724.1 |
| chr225 | 254520 | 288000 | NC_008724.1 |
| chr226 | 0 | 8520 | NC_007580.2 |
| chr226 | 8640 | 10940 | NC_007580.2 |
| chr228 | 0 | 14520 | NC_009020.1 |
| chr228 | 14640 | 30482 | NC_009020.1 |
| chr229 | 0 | 3245 | NC_009225.1 |
| chr230 | 0 | 5040 | NC_009238.1 |
| chr232 | 0 | 29880 | NC_009333.1 |
| chr232 | 30000 | 125280 | NC_009333.1 |
| chr232 | 125520 | 125640 | NC_009333.1 |
| chr232 | 125760 | 126240 | NC_009333.1 |
| chr232 | 126360 | 137969 | NC_009333.1 |
| chr233 | 0 | 7590 | NC_003976.2 |
| chr234 | 0 | 15486 | NC_009489.1 |
| chr235 | 0 | 11966 | NC_009527.1 |
| chr236 | 0 | 11930 | NC_009528.1 |
| chr237 | 0 | 5229 | NC_009539.1 |
| chr238 | 0 | 15180 | NC_009640.1 |
| chr239 | 0 | 28203 | NC_009657.1 |
| chr241 | 0 | 3360 | NC_009825.1 |
| chr241 | 3480 | 9355 | NC_009825.1 |
| chr242 | 0 | 9343 | NC_009826.1 |
| chr243 | 0 | 9711 | NC_009823.1 |
| chr244 | 0 | 9628 | NC_009827.1 |
| chr245 | 0 | 9456 | NC_009824.1 |
| chr247 | 0 | 19111 | NC_001608.3 |
| chr248 | 0 | 10723 | NC_001474.2 |
| chr249 | 0 | 5075 | NC_009951.1 |
| chr250 | 0 | 27165 | NC_009988.1 |
| chr251 | 0 | 10707 | NC_001475.2 |
| chr252 | 0 | 3535 | NC_010248.1 |
| chr256 | 0 | 3341 | NC_010247.1 |
| chr257 | 0 | 10837 | NC_008604.2 |
| chr258 | 0 | 7326 | NC_010329.1 |
| chr259 | 0 | 28476 | NC_010436.1 |
| chr260 | 0 | 28326 | NC_010437.1 |
| chr261 | 0 | 28773 | NC_010438.1 |
| chr263 | 0 | 8115 | NC_009448.2 |
| chr265 | 0 | 7141 | NC_010702.1 |
| chr268 | 0 | 3410 | NC_010757.1 |
| chr269 | 0 | 3474 | NC_010758.1 |
| chr270 | 0 | 2269 | NC_010746.1 |
| chr271 | 0 | 7961 | NC_010810.1 |
| chr272 | 0 | 13111 | NC_010820.1 |
| chr273 | 0 | 12972 | NC_010819.1 |
| chr274 | 0 | 35083 | NC_010956.1 |
| chr275 | 0 | 35343 | NC_011203.1 |
| chr276 | 0 | 5081 | NC_011310.1 |
| chr277 | 0 | 6171 | NC_011400.1 |
| chr278 | 0 | 8913 | NC_011546.1 |
| chr279 | 0 | 8210 | NC_011829.1 |
| chr280 | 0 | 8791 | NC_011800.1 |
| chr281 | 120 | 5196 | NC_012042.1 |
| chr282 | 120 | 7680 | NC_001664.2 |
| chr282 | 8040 | 131040 | NC_001664.2 |
| chr282 | 132120 | 151320 | NC_001664.2 |
| chr282 | 151440 | 158880 | NC_001664.2 |
| chr283 | 0 | 2140 | NC_012126.1 |
| chr284 | 0 | 7149 | NC_012213.1 |
| chr285 | 0 | 7722 | NC_012437.1 |
| chr286 | 0 | 7346 | NC_012485.1 |
| chr287 | 0 | 7227 | NC_012486.1 |
| chr288 | 0 | 10794 | NC_012532.1 |
| chr289 | 0 | 10723 | NC_012533.1 |
| chr290 | 0 | 10941 | NC_012534.1 |
| chr291 | 0 | 5242 | NC_012564.1 |
| chr292 | 0 | 10865 | NC_012671.1 |
| chr293 | 0 | 10814 | NC_012735.1 |
| chr294 | 0 | 3189 | NC_012776.1 |
| chr295 | 0 | 6580 | NC_012798.1 |
| chr296 | 0 | 5640 | NC_012801.1 |
| chr296 | 5760 | 7205 | NC_012801.1 |
| chr297 | 0 | 7215 | NC_012802.1 |
| chr298 | 0 | 600 | NC_012950.1 |
| chr298 | 720 | 2160 | NC_012950.1 |
| chr298 | 2520 | 2760 | NC_012950.1 |
| chr298 | 2880 | 4800 | NC_012950.1 |
| chr298 | 4920 | 5040 | NC_012950.1 |
| chr298 | 5160 | 5400 | NC_012950.1 |
| chr298 | 5760 | 5880 | NC_012950.1 |
| chr298 | 6000 | 6120 | NC_012950.1 |
| chr298 | 6960 | 7080 | NC_012950.1 |
| chr298 | 7200 | 9000 | NC_012950.1 |
| chr298 | 9240 | 19320 | NC_012950.1 |
| chr298 | 19440 | 19920 | NC_012950.1 |
| chr298 | 20040 | 20640 | NC_012950.1 |
| chr298 | 21000 | 21600 | NC_012950.1 |
| chr298 | 21720 | 21840 | NC_012950.1 |
| chr298 | 21960 | 22080 | NC_012950.1 |
| chr298 | 22440 | 22680 | NC_012950.1 |
| chr298 | 23160 | 27600 | NC_012950.1 |
| chr298 | 27720 | 27840 | NC_012950.1 |
| chr298 | 28080 | 28200 | NC_012950.1 |
| chr298 | 28320 | 28680 | NC_012950.1 |
| chr298 | 28920 | 29040 | NC_012950.1 |
| chr298 | 29160 | 29880 | NC_012950.1 |
| chr298 | 30000 | 30240 | NC_012950.1 |
| chr298 | 30480 | 30953 | NC_012950.1 |
| chr299 | 0 | 34920 | NC_012959.1 |
| chr300 | 0 | 11064 | NC_012932.1 |
| chr301 | 0 | 7184 | NC_013035.1 |
| chr302 | 0 | 3383 | NC_013057.1 |
| chr304 | 0 | 10815 | NC_009026.2 |
| chr306 | 0 | 5168 | NC_013439.1 |
| chr307 | 0 | 5112 | NC_013796.1 |
| chr308 | 0 | 5104 | NC_012729.2 |
| chr309 | 0 | 10874 | NC_009029.2 |
| chr310 | 0 | 10755 | NC_009028.2 |
| chr311 | 0 | 2064 | NC_014072.1 |
| chr312 | 0 | 3629 | NC_014073.1 |
| chr313 | 0 | 2910 | NC_014068.1 |
| chr314 | 0 | 3729 | NC_014074.1 |
| chr315 | 0 | 3759 | NC_014075.1 |
| chr316 | 0 | 3770 | NC_014076.1 |
| chr317 | 0 | 3690 | NC_014069.1 |
| chr318 | 0 | 2878 | NC_014070.1 |
| chr319 | 0 | 2797 | NC_014071.1 |
| chr320 | 0 | 3899 | NC_014077.1 |
| chr321 | 0 | 1080 | NC_014078.1 |
| chr321 | 1200 | 1440 | NC_014078.1 |
| chr321 | 1800 | 3000 | NC_014078.1 |
| chr321 | 3120 | 3808 | NC_014078.1 |
| chr322 | 0 | 3798 | NC_014079.1 |
| chr323 | 0 | 3736 | NC_014080.1 |
| chr324 | 0 | 3748 | NC_014081.1 |
| chr325 | 120 | 2880 | NC_014082.1 |
| chr326 | 0 | 3763 | NC_014083.1 |
| chr327 | 0 | 3790 | NC_014084.1 |
| chr328 | 0 | 3371 | NC_014085.1 |
| chr329 | 0 | 2640 | NC_014086.1 |
| chr330 | 0 | 3718 | NC_014087.1 |
| chr331 | 0 | 2760 | NC_014088.1 |
| chr332 | 0 | 2908 | NC_014089.1 |
| chr333 | 0 | 2785 | NC_014090.1 |
| chr334 | 0 | 3818 | NC_014091.1 |
| chr335 | 0 | 3253 | NC_014093.1 |
| chr336 | 0 | 3705 | NC_014094.1 |
| chr337 | 0 | 2897 | NC_014095.1 |
| chr338 | 0 | 3787 | NC_014096.1 |
| chr339 | 0 | 2856 | NC_014097.1 |
| chr340 | 0 | 7342 | NC_014185.1 |
| chr341 | 0 | 18935 | NC_014372.1 |
| chr342 | 0 | 5232 | NC_014361.1 |
| chr343 | 0 | 18940 | NC_014373.1 |
| chr344 | 0 | 3360 | NC_009424.4 |
| chr344 | 4440 | 5159 | NC_009424.4 |
| chr346 | 0 | 4952 | NC_014407.1 |
| chr347 | 0 | 4926 | NC_014406.1 |
| chr348 | 0 | 4286 | NC_014468.1 |
| chr349 | 0 | 7181 | NC_014469.1 |
| chr350 | 0 | 29276 | NC_014470.1 |
| chr351 | 0 | 8126 | NC_014474.1 |
| chr352 | 0 | 7905 | NC_001526.2 |
| chr354 | 0 | 3322 | NC_014480.2 |
| chr355 | 0 | 2520 | NC_014092.2 |
| chr356 | 0 | 5086 | NC_014743.1 |
| chr357 | 0 | 4981 | NC_004764.2 |
| chr358 | 0 | 1703 | NC_014929.1 |
| chr359 | 0 | 7309 | NC_014956.1 |
| chr360 | 0 | 7125 | NC_014955.1 |
| chr361 | 0 | 7182 | NC_014954.1 |
| chr362 | 0 | 7219 | NC_014953.1 |
| chr363 | 0 | 7259 | NC_014952.1 |
| chr364 | 0 | 2164 | NC_015212.1 |
| chr365 | 0 | 5026 | NC_015150.1 |
| chr366 | 0 | 35499 | NC_015225.1 |
| chr370 | 0 | 7310 | NC_015521.1 |
| chr371 | 0 | 9762 | NC_001545.2 |
| chr372 | 0 | 7632 | NC_015691.1 |
| chr373 | 0 | 7686 | NC_015692.1 |
| chr374 | 0 | 3480 | NC_015783.1 |
| chr374 | 3600 | 3725 | NC_015783.1 |
| chr375 | 0 | 7749 | NC_015934.1 |
| chr376 | 0 | 7753 | NC_015940.1 |
| chr377 | 0 | 7693 | NC_015941.1 |
| chr378 | 0 | 31616 | NC_015932.1 |
| chr379 | 0 | 6543 | NC_015935.1 |
| chr381 | 0 | 18927 | NC_016144.1 |
| chr382 | 0 | 5596 | NC_000883.2 |
| chr384 | 0 | 218041 | NC_016154.1 |
| chr385 | 0 | 7326 | NC_016157.1 |
| chr386 | 0 | 3960 | NC_016744.1 |
| chr386 | 4080 | 5065 | NC_016744.1 |
| chr387 | 0 | 31681 | NC_016895.1 |
| chr388 | 0 | 6707 | NC_016896.1 |
| chr389 | 0 | 6119 | NC_016155.1 |
| chr390 | 0 | 10791 | NC_016997.1 |
| chr391 | 0 | 10990 | NC_015843.2 |
| chr392 | 0 | 7696 | NC_017936.1 |
| chr393 | 0 | 15276 | NC_017937.1 |
| chr394 | 0 | 4987 | NC_017982.1 |
| chr395 | 0 | 7293 | NC_017993.1 |
| chr396 | 0 | 7319 | NC_017994.1 |
| chr397 | 0 | 7236 | NC_017995.1 |
| chr398 | 0 | 7341 | NC_017996.1 |
| chr399 | 0 | 7271 | NC_017997.1 |
| chr401 | 0 | 10733 | NC_017086.1 |
| chr402 | 0 | 5421 | NC_017085.1 |
| chr403 | 0 | 4939 | X262162.1 |
| chr405 | 0 | 6796 | NC_018382.1 |
| chr406 | 0 | 834 | gment S, ″gi |
| chr407 | 0 | 843 | 1829611ref |
| chr408 | 0 | 6863 | 50″, segmen |
| chr409 | 0 | 860 | ″, segment |
| chr410 | 0 | 3368 | NC_018481.1 |
| chr411 | 0 | 6922 | NC_018482.1 |
| chr412 | 0 | 11902 | NC_018629.1 |
| chr413 | 0 | 4939 | X259273.1 |
| chr414 | 0 | 6531 | NC_018669.1 |
| chr415 | 0 | 6838 | NC_018702.1 |
| chr417 | 0 | 28494 | NC_018871.1 |
| chr418 | 0 | 7212 | NC_019023.1 |
| chr419 | 0 | 6581 | NC_019026.1 |
| chr420 | 0 | 6518 | NC_019027.1 |
| chr421 | 0 | 6124 | NC_019028.1 |
| chr422 | 0 | 6460 | NC_019494.1 |
| chr423 | 0 | 15048 | NC_019531.1 |
| chr424 | 0 | 5157 | NC_019844.1 |
| chr425 | 0 | 5140 | NC_019850.1 |
| chr426 | 0 | 5087 | NC_019851.1 |
| chr427 | 0 | 5273 | NC_019853.1 |
| chr428 | 0 | 5333 | NC_019855.1 |
| chr429 | 0 | 5349 | NC_019856.1 |
| chr430 | 0 | 4994 | NC_019857.1 |
| chr431 | 0 | 4970 | NC_019858.1 |
| chr432 | 0 | 4899 | NC_020065.1 |
| chr433 | 0 | 4914 | NC_020066.1 |
| chr434 | 0 | 5372 | NC_020067.1 |
| chr435 | 0 | 5294 | NC_020068.1 |
| chr436 | 0 | 5213 | NC_020069.1 |
| chr437 | 0 | 5136 | NC_020070.1 |
| chr438 | 0 | 5176 | NC_020071.1 |
| chr439 | 0 | 4775 | JX463184.1 |
| chr440 | 0 | 4776 | NC_020106.1 |
| chr441 | 0 | 2315 | NC_015630.1 |
| chr442 | 0 | 34302 | NC_020485.1 |
| chr443 | 0 | 2912 | NC_020498.1 |
| chr444 | 0 | 11120 | NC_020805.1 |
| chr445 | 0 | 12016 | NC_020807.1 |
| chr446 | 0 | 11918 | NC_020808.1 |
| chr447 | 0 | 11980 | NC_020809.1 |
| chr448 | 0 | 11160 | NC_020810.1 |
| chr448 | 11280 | 11976 | NC_020810.1 |
| chr449 | 0 | 3230 | NC_020881.1 |
| chr450 | 0 | 5033 | NC_020890.1 |
| chr451 | 0 | 7320 | NC_021069.1 |
| chr451 | 7440 | 7800 | NC_021069.1 |
| chr451 | 7920 | 9480 | NC_021069.1 |
| chr451 | 9600 | 9720 | NC_021069.1 |
| chr451 | 9840 | 10865 | NC_021069.1 |
| chr452 | 0 | 8879 | NC_021153.1 |
| chr453 | 0 | 34391 | NC_021168.1 |
| chr454 | 0 | 36838 | NC_020487.1 |
| chr455 | 0 | 1798 | NC_021206.1 |
| chr456 | 0 | 7286 | NC_021483.1 |
| chr458 | 0 | 1832 | NC_021568.1 |
| chr461 | 0 | 17052 | NC_021928.1 |
| chr462 | 0 | 18234 | NC_001906.3 |
| chr464 | 0 | 7228 | NC_022095.1 |
| chr465 | 0 | 5270 | NC_004763.2 |
| chr466 | 0 | 28035 | NC_022103.1 |
| chr467 | 0 | 6795 | NC_022249.1 |
| chr468 | 0 | 31967 | NC_022266.1 |
| chr469 | 0 | 5013 | NC_019854.2 |
| chr471 | 0 | 5722 | NC_022519.1 |
| chr472 | 0 | 3303 | NC_022631.1 |
| chr473 | 0 | 1560 | NC_022755.1 |
| chr473 | 1800 | 3960 | NC_022755.1 |
| chr473 | 4080 | 4320 | NC_022755.1 |
| chr473 | 4440 | 10692 | NC_022755.1 |
| chr474 | 0 | 7228 | NC_022892.1 |
| chr475 | 0 | 10943 | NC_018705.3 |
| chr476 | 0 | 5084 | NC_023008.1 |
| chr477 | 0 | 6253 | NC_023629.1 |
| chr478 | 0 | 6301 | NC_023630.1 |
| chr479 | 0 | 6300 | NC_023631.1 |
| chr480 | 0 | 6317 | NC_023632.1 |
| chr481 | 0 | 6376 | NC_023635.1 |
| chr482 | 0 | 6500 | NC_023636.1 |
| chr483 | 0 | 6318 | NC_023674.1 |
| chr484 | 0 | 6120 | NC_023675.1 |
| chr484 | 6240 | 6600 | NC_023675.1 |
| chr485 | 0 | 1790 | NC_023874.1 |
| chr486 | 0 | 7314 | NC_023891.1 |
| chr487 | 0 | 7802 | NC_023984.1 |
| chr488 | 0 | 5108 | F954417.1 |
| chr489 | 0 | 983 | NC_024075.1 |
| chr490 | 0 | 8914 | NC_024296.1 |
| chr491 | 0 | 6233 | NC_024297.1 |
| chr492 | 0 | 111000 | NC_024306.1 |
| chr492 | 111120 | 149459 | NC_024306.1 |
| chr493 | 0 | 9525 | NC_024377.1 |
| chr494 | 0 | 3368 | NC_024443.1 |
| chr495 | 0 | 3377 | NC_024444.1 |
| chr496 | 0 | 3149 | NC_024445.1 |
| chr497 | 0 | 1855 | NC_021707.2 |
| chr498 | 0 | 6577 | NC_024472.1 |
| chr499 | 0 | 1772 | NC_024496.1 |
| chr500 | 0 | 6530 | NC_024498.1 |
| chr501 | 0 | 1766 | LK931491.1 |
| chr502 | 0 | 1766 | LK931492.1 |
| chr503 | 0 | 1440 | NC_024689.1 |
| chr503 | 1560 | 2152 | NC_024689.1 |
| chr504 | 0 | 2121 | NC_024690.1 |
| chr505 | 0 | 2259 | NC_024691.1 |
| chr506 | 0 | 2836 | NC_024694.1 |
| chr507 | 0 | 6598 | NC_024701.1 |
| chr508 | 0 | 8884 | NC_024778.1 |
| chr509 | 0 | 2169 | NC_024890.1 |
| chr510 | 0 | 2149 | NC_024891.1 |
| chr511 | 0 | 2367 | NC_024908.1 |
| chr512 | 0 | 31491 | NC_025217.1 |
| chr513 | 0 | 11900 | NC_025251.1 |
| chr514 | 0 | 18530 | NC_025256.1 |
| chr515 | 0 | 11139 | NC_025341.1 |
| chr516 | 0 | 7353 | NC_025346.1 |
| chr517 | 0 | 12045 | NC_025365.1 |
| chr518 | 0 | 5309 | NC_025370.1 |
| chr519 | 0 | 12278 | NC_025377.1 |
| chr520 | 0 | 15624 | NC_025403.1 |
| chr521 | 0 | 15504 | NC_025404.1 |
| chr522 | 0 | 6584 | NC_025409.1 |
| chr523 | 0 | 170101 | NC_001659.2 |
| chr524 | 0 | 12240 | NC_003092.2 |
| chr524 | 12360 | 13920 | NC_003092.2 |
| chr524 | 14040 | 14160 | NC_003092.2 |
| chr524 | 14280 | 14760 | NC_003092.2 |
| chr524 | 15000 | 15120 | NC_003092.2 |
| chr524 | 15240 | 15717 | NC_003092.2 |
| chr525 | 0 | 26640 | NC_025678.1 |
| chr525 | 26760 | 32661 | NC_025678.1 |
| chr526 | 0 | 5307 | NC_001515.2 |
| chr527 | 0 | 5372 | NC_001663.2 |
| chr528 | 0 | 5387 | NC_010277.2 |
| chr529 | 0 | 140880 | GQ466044.1 |
| chr529 | 141000 | 235703 | GQ466044.1 |
| chr531 | 0 | 236219 | GQ221973.1 |
| chr533 | 0 | 235154 | GQ221974.1 |
| chr535 | 0 | 236428 | KP745728.1 |
| chr538 | 0 | 10807 | KU681082.3 |
| chrA1 | 0 | 1750 | A06324_1 |
| chrA10 | 0 | 4200 | NC_004109_1 |
| chrA10 | 4320 | 4527 | NC_004109_1 |
| chrA100 | 0 | 526 | KX545358_1 |
| chrA101 | 0 | 507 | KX545361_1 |
| chrA102 | 0 | 513 | KX545364_1 |
| chrA103 | 0 | 558 | KX545366_1 |
| chrA104 | 120 | 1587 | KY565593_1 |
| chrA105 | 120 | 1569 | KY565603_1 |
| chrA106 | 120 | 1567 | KY565665_1 |
| chrA107 | 0 | 1217 | KX645743_1 |
| chrA108 | 0 | 1605 | KX645765_1 |
| chrA109 | 0 | 1752 | MF588762_1 |
| chrA11 | 0 | 1884 | NC_004180_1 |
| chrA110 | 0 | 1863 | MF588765_1 |
| chrA111 | 0 | 1670 | MF588778_1 |
| chrA112 | 0 | 2056 | MF588781_1 |
| chrA113 | 0 | 1533 | MG921179_1 |
| chrA12 | 0 | 3048 | NC_004217_1 |
| chrA13 | 0 | 3035 | NC_004212_1 |
| chrA14 | 0 | 3402 | NC_004296_1 |
| chrA15 | 0 | 3671 | NC_003467_2 |
| chrA16 | 0 | 890 | NC_004906_1 |
| chrA17 | 0 | 1714 | NC_004908_1 |
| chrA18 | 0 | 1418 | NC_004909_1 |
| chrA19 | 0 | 3000 | NC_005220_1 |
| chrA19 | 3109 | 3229 | NC_005220_1 |
| chrA2 | 0 | 1527 | A09292_1 |
| chrA20 | 0 | 3480 | NC_005223_1 |
| chrA21 | 0 | 3696 | NC_005215_1 |
| chrA22 | 0 | 3694 | NC_005228_1 |
| chrA23 | 0 | 4320 | NC_005775_1 |
| chrA24 | 0 | 3393 | NC_005894_1 |
| chrA25 | 0 | 3366 | NC_006317_1 |
| chrA26 | 0 | 3616 | NC_006437_1 |
| chrA27 | 0 | 1560 | NC_006506_1 |
| chrA28 | 0 | 2525 | NC_007026_1 |
| chrA29 | 0 | 624 | DQ091857_1 |
| chrA3 | 960 | 2040 | L36108_1 |
| chrA30 | 0 | 865 | NC_007364_1 |
| chrA31 | 0 | 2341 | NC_007357_1 |
| chrA32 | 0 | 2341 | NC_007358_1 |
| chrA33 | 0 | 2233 | NC_007359_1 |
| chrA34 | 0 | 1760 | NC_007362_1 |
| chrA35 | 0 | 1027 | NC_007363_1 |
| chrA36 | 0 | 1773 | NC_007374_1 |
| chrA37 | 0 | 1762 | NC_007366_1 |
| chrA38 | 0 | 1458 | NC_007361_1 |
| chrA39 | 0 | 5366 | NC_005300_2 |
| chrA4 | 0 | 1313 | X67160_1 |
| chrA40 | 0 | 1287 | NC_007553_1 |
| chrA41 | 0 | 360 | NC_007543_1 |
| chrA41 | 600 | 1350 | NC_007543_1 |
| chrA42 | 0 | 3055 | NC_007737_1 |
| chrA43 | 0 | 3380 | NC_007903_1 |
| chrA44 | 0 | 3366 | NC_007905_1 |
| chrA45 | 0 | 4309 | NC_009895_1 |
| chrA46 | 0 | 1533 | DJ042838_1 |
| chrA47 | 0 | 3339 | NC_010256_1 |
| chrA48 | 0 | 3332 | NC_010254_1 |
| chrA49 | 0 | 3343 | NC_010253_1 |
| chrA5 | 0 | 1680 | X67161_1 |
| chrA50 | 0 | 3357 | NC_010562_1 |
| chrA51 | 0 | 3316 | NC_010700_1 |
| chrA52 | 0 | 3480 | NC_010708_1 |
| chrA53 | 0 | 3533 | NC_010701_1 |
| chrA54 | 0 | 1503 | FM876121_1 |
| chrA55 | 0 | 480 | GQ228066_1 |
| chrA55 | 840 | 1080 | GQ228066_1 |
| chrA55 | 1320 | 1498 | GQ228066_1 |
| chrA56 | 0 | 1500 | GQ479012_1 |
| chrA57 | 0 | 1509 | GQ479020_1 |
| chrA58 | 0 | 1515 | GQ487711_1 |
| chrA59 | 0 | 1599 | GQ288789_1 |
| chrA6 | 0 | 2160 | U40822_1 |
| chrA6 | 2520 | 3891 | U40822_1 |
| chrA60 | 0 | 3885 | NC_014396_1 |
| chrA61 | 0 | 1666 | NC_014527_1 |
| chrA62 | 0 | 713 | HQ724330_1 |
| chrA63 | 0 | 1440 | NC_015373_1 |
| chrA63 | 1560 | 4326 | NC_015373_1 |
| chrA64 | 0 | 4080 | NC_015411_1 |
| chrA64 | 4200 | 4403 | NC_015411_1 |
| chrA65 | 0 | 4185 | NC_015450_1 |
| chrA66 | 0 | 1518 | JN104068_1 |
| chrA67 | 0 | 1527 | JN104073_1 |
| chrA68 | 0 | 1356 | NC_011509_2 |
| chrA69 | 0 | 3377 | NC_016152_1 |
| chrA7 | 0 | 1778 | NC_002017_1 |
| chrA70 | 0 | 1590 | JN874416_1 |
| chrA71 | 0 | 1518 | HE793049_1 |
| chrA72 | 0 | 1521 | JQ963486_1 |
| chrA73 | 0 | 1509 | HE820129_1 |
| chrA74 | 0 | 3378 | NC_018138_1 |
| chrA75 | 0 | 1512 | HE962401_1 |
| chrA76 | 0 | 1503 | HE963119_1 |
| chrA77 | 0 | 1506 | HE963156_1 |
| chrA78 | 0 | 1050 | JQ976760_1 |
| chrA79 | 0 | 4335 | NC_018459_1 |
| chrA8 | 0 | 4458 | NC_001926_1 |
| chrA80 | 0 | 4200 | NC_018466_1 |
| chrA81 | 0 | 4314 | NC_018467_1 |
| chrA82 | 0 | 4417 | NC_018478_1 |
| chrA83 | 0 | 3419 | NC_018710_1 |
| chrA84 | 0 | 1269 | NC_021544_1 |
| chrA85 | 0 | 1267 | NC_021588_1 |
| chrA86 | 0 | 1314 | NC_021635_1 |
| chrA87 | 0 | 1440 | NC_022037_1 |
| chrA87 | 1552 | 1672 | NC_022037_1 |
| chrA88 | 0 | 3720 | NC_023633_1 |
| chrA88 | 3960 | 4088 | NC_023633_1 |
| chrA89 | 0 | 1707 | KU721789_1 |
| chrA9 | 0 | 4349 | NC_003696_1 |
| chrA90 | 0 | 1272 | KU721801_1 |
| chrA91 | 0 | 1575 | KU550602_1 |
| chrA92 | 0 | 1551 | KU163569_1 |
| chrA93 | 0 | 785 | KU951267_1 |
| chrA94 | 0 | 1524 | KY348861_1 |
| chrA95 | 0 | 550 | KX545349_1 |
| chrA96 | 0 | 525 | KX545350_1 |
| chrA97 | 0 | 527 | KX545353_1 |
| chrA98 | 0 | 531 | KX545356_1 |
| chrA99 | 0 | 515 | KX545357_1 |
| chrB1 | 0 | 29903 | NC_045512.2 |

TABLE 2
Percent of genome length covered by NCBI genome ID and Viral Common name.

| | | Genome | | Percentage |
|---|---|---|---|---|
| | | length | | of viral |
| | | covered by | Genome | genome |
| Genome ID | Virus | probes | length | covered |
| NC_002208.1 | Influenza B virus RNA 5, complete sequence″ | 1841 | 14452 | 12.74% |
| NC_001348.1 | Human herpesvirus 3, complete genome″ | 124764 | 124884 | 99.90% |
| NC_001354.1 | Human papillomavirus type 41, complete genome″ | 7614 | 7614 | 100.00% |
| NC_001355.1 | Human papillomavirus type 6b, complete genome″ | 7902 | 7902 | 100.00% |
| NC_001358.1 | Parvovirus H1, complete genome″ | 5176 | 5176 | 100.00% |
| NC_001364.1 | Simian foamy virus, complete genome″ | 13246 | 13246 | 100.00% |
| NC_001405.1 | Human adenovirus C, complete genome″ | 35937 | 35937 | 100.00% |
| NC_001430.1 | Human enterovirus D, complete genome″ | 7030 | 7390 | 95.13% |
| NC_001434.1 | Hepatitis E virus, complete genome″ | 7176 | 7176 | 100.00% |
| NC_001436.1 | Human T-lymphotropic virus 1, complete genome″ | 8507 | 8507 | 100.00% |
| NC_001437.1 | Japanese encephalitis virus, genome″ | 10976 | 10976 | 100.00% |
| NC_001442.1 | Bovine polyomavirus, complete genome″ | 4697 | 4697 | 100.00% |
| NC_001449.1 | Venezuelan equine encephalitis virus, complete | 11444 | 11444 | 100.00% |
| | genome″ | | | |
| NC_001454.1 | Human adenovirus F, complete genome″ | 34214 | 34214 | 100.00% |
| NC_001457.1 | Human papillomavirus type 4, complete genome″ | 7353 | 7353 | 100.00% |
| NC_001458.1 | Human papillomavirus type 63, complete genome″ | 7348 | 7348 | 100.00% |
| NC_001460.1 | Human adenovirus A, complete genome″ | 34125 | 34125 | 100.00% |
| NC_001463.1 | Caprine arthritis-encephalitis virus, complete genome″ | 9189 | 9189 | 100.00% |
| NC_001472.1 | Human enterovirus B, complete genome″ | 7389 | 7389 | 100.00% |
| NC_001477.1 | Dengue virus 1, complete genome″ | 10735 | 10735 | 100.00% |
| NC_001479.1 | Encephalomyocarditis virus, complete genome″ | 7835 | 7835 | 100.00% |
| NC_001488.1 | Human T-lymphotropic virus 2, complete genome″ | 8952 | 8952 | 100.00% |
| NC_001489.1 | Hepatitis A virus, complete genome″ | 7478 | 7478 | 100.00% |
| NC_001490.1 | Human rhinovirus 14, complete genome″ | 7212 | 7212 | 100.00% |
| NC_001498.1 | Measles virus, complete genome″ | 15894 | 15894 | 100.00% |
| NC_001512.1 | O'nyong-nyong virus, complete genome″ | 11835 | 11835 | 100.00% |
| NC_001531.1 | Human papillomavirus - 5, complete genome″ | 7746 | 7746 | 100.00% |
| NC_001538.1 | BK polyomavirus, complete genome″ | 5153 | 5153 | 100.00% |
| NC_001542.1 | Rabies virus, complete genome″ | 11932 | 11932 | 100.00% |
| NC_001549.1 | Simian immunodeficiency virus, complete genome″ | 9623 | 9623 | 100.00% |
| NC_001550.1 | Mason-Pfizer monkey virus, complete genome″ | 8437 | 8557 | 98.60% |
| NC_001552.1 | Sendai virus, complete genome″ | 15384 | 15384 | 100.00% |
| NC_001560.1 | Vesicular stomatitis Indiana virus, complete genome″ | 11161 | 11161 | 100.00% |
| NC_001564.1 | Cell fusing agent virus, complete genome″ | 10695 | 10695 | 100.00% |
| NC_002031.1 | Yellow fever virus, complete genome″ | 10862 | 10862 | 100.00% |
| NC_001576.1 | Human papillomavirus type 10, complete genome″ | 7919 | 7919 | 100.00% |
| NC_001583.1 | Human papillomavirus type 26, complete genome″ | 7855 | 7855 | 100.00% |
| NC_001586.1 | Human papillomavirus type 32, complete genome″ | 7961 | 7961 | 100.00% |
| NC_001587.1 | Human papillomavirus type 34, complete genome″ | 7603 | 7723 | 98.45% |
| NC_001591.1 | Human papillomavirus type 49, complete genome″ | 7560 | 7560 | 100.00% |
| NC_001593.1 | Human papillomavirus type 53, complete genome″ | 7856 | 7856 | 100.00% |
| NC_001595.1 | Human papillomavirus type 7, complete genome″ | 8027 | 8027 | 100.00% |
| NC_001596.1 | Human papillomavirus type 9, complete genome″ | 7434 | 7434 | 100.00% |
| NC_001607.1 | Borna disease virus, complete genome″ | 8910 | 8910 | 100.00% |
| NC_001611.1 | Variola virus, complete genome″ | 185338 | 185578 | 99.87% |
| NC_001612.1 | Human enterovirus A, complete genome″ | 7413 | 7413 | 100.00% |
| NC_001617.1 | Human rhinovirus 89, complete genome″ | 7152 | 7152 | 100.00% |
| NC_001669.1 | Simian virus 40, complete genome″ | 5243 | 5243 | 100.00% |
| NC_001672.1 | Tick-borne encephalitis virus, complete genome″ | 11141 | 11141 | 100.00% |
| NC_001690.1 | Human papillomavirus type 48, complete genome″ | 7100 | 7100 | 100.00% |
| NC_001691.1 | Human papillomavirus type 50, complete genome″ | 7184 | 7184 | 100.00% |
| NC_001693.1 | Human papillomavirus type 60, complete genome″ | 7313 | 7313 | 100.00% |
| NC_001699.1 | JC polyomavirus, complete genome″ | 5130 | 5130 | 100.00% |
| NC_001710.1 | GB virus C/Hepatitis G virus, complete genome″ | 9360 | 9360 | 100.00% |
| NC_001722.1 | Human immunodeficiency virus 2, complete genome″ | 10359 | 10359 | 100.00% |
| NC_001729.1 | Adeno-associated virus - 3, complete genome″ | 4726 | 4726 | 100.00% |
| NC_001781.1 | Human respiratory syncytial virus, complete genome″ | 15225 | 15225 | 100.00% |
| NC_001786.1 | Barmah Forest virus, complete genome″ | 11248 | 11488 | 97.91% |
| NC_001798.1 | Human herpesvirus 2, complete genome″ | 153906 | 154746 | 99.46% |
| NC_001802.1 | Human immunodeficiency virus 1, complete genome″ | 9181 | 9181 | 100.00% |
| NC_001803.1 | Respiratory syncytial virus, complete genome″ | 15191 | 15191 | 100.00% |
| NC_001806.1 | Human herpesvirus 1, complete genome″ | 150600 | 152261 | 98.91% |
| NC_001809.1 | Louping ill virus, complete genome″ | 10871 | 10871 | 100.00% |
| NC_001815.1 | Simian T-lymphotropic virus 2, complete genome″ | 8735 | 8855 | 98.64% |
| NC_001829.1 | Adeno-associated virus - 4, complete genome″ | 4767 | 4767 | 100.00% |
| NC_001897.1 | Human parechovirus, genome″ | 7348 | 7348 | 100.00% |
| NC_001918.1 | Aichi virus, complete genome″ | 8251 | 8251 | 100.00% |
| NC_001921.1 | Canine distemper virus, complete genome″ | 15690 | 15690 | 100.00% |
| NC_001943.1 | Human astrovirus, complete genome″ | 6813 | 6813 | 100.00% |
| NC_001989.1 | Bovine respiratory syncytial virus, complete genome″ | 15140 | 15140 | 100.00% |
| NC_002077.1 | Adeno-associated virus - 1, complete genome″ | 4718 | 4718 | 100.00% |
| NC_000858.1 | Simian T-lymphotropic virus 1, complete genome″ | 9028 | 9028 | 100.00% |
| NC_000898.1 | Human herpesvirus 6B, complete genome″ | 159960 | 162114 | 98.67% |
| NC_000940.1 | Porcine enteric sapovirus, complete genome″ | 7320 | 7320 | 100.00% |
| NC_000943.1 | Murray Valley encephalitis virus, complete genome″ | 11014 | 11014 | 100.00% |
| NC_002161.1 | Bovine parainfluenza virus 3, complete genome″ | 15456 | 15456 | 100.00% |
| NC_002195.1 | Torque teno mini virus 9, complete genome″ | 2760 | 2760 | 100.00% |
| NC_002199.1 | Tupaia paramyxovirus, complete genome″ | 17904 | 17904 | 100.00% |
| NC_002470.1 | Turkey astrovirus, complete genome″ | 6480 | 6960 | 93.10% |
| NC_002200.1 | Mumps virus, complete genome″ | 15384 | 15384 | 100.00% |
| NC_001544.1 | Ross River virus, complete genome″ | 9497 | 11657 | 81.47% |
| NC_002549.1 | Zaire ebolavirus isolate Ebola | 18959 | 18959 | 100.00% |
| | virus/H. sapiens-tc/COD/1976/Yambuku-May | | | |
| NC_002551.1 | Vesicular exanthema of swine virus, complete | 8284 | 8284 | 100.00% |
| | genome″ | | | |
| NC_001796.2 | Human parainfluenza virus 3, complete genome″ | 15462 | 15462 | 100.00% |
| NC_001563.2 | West Nile virus, complete genome″ | 10962 | 10962 | 100.00% |
| NC_002617.1 | Newcastle disease virus B1, complete genome″ | 15186 | 15186 | 100.00% |
| NC_002640.1 | Dengue virus 4, complete genome″ | 10649 | 10649 | 100.00% |
| NC_002645.1 | Human coronavirus 229E, complete genome″ | 27317 | 27317 | 100.00% |
| NC_002058.3 | Poliovirus, complete genome″ | 7440 | 7440 | 100.00% |
| NC_002657.1 | Classical swine fever virus, complete genome″ | 12301 | 12301 | 100.00% |
| NC_001653.2 | Hepatitis delta virus, complete genome″ | 1682 | 1682 | 100.00% |
| NC_002728.1 | Nipah virus, complete genome″ | 18246 | 18246 | 100.00% |
| NC_003043.1 | Avian paramyxovirus 6, complete genome″ | 16236 | 16236 | 100.00% |
| NC_003243.1 | Australian bat lyssavirus, complete genome″ | 11822 | 11822 | 100.00% |
| NC_003310.1 | Monkeypox virus Zaire-96-I-16, complete genome″ | 196018 | 196858 | 99.57% |
| NC_003323.1 | Simian T-lymphotropic virus 3, complete genome″ | 8678 | 8918 | 97.31% |
| NC_003389.1 | Swinepox virus, complete genome″ | 146454 | 146454 | 100.00% |
| NC_003417.1 | Mayaro virus, complete genome″ | 11411 | 11411 | 100.00% |
| NC_003443.1 | Human parainfluenza virus 2, complete genome″ | 15646 | 15646 | 100.00% |
| NC_003461.1 | Human parainfluenza virus 1, complete genome″ | 15600 | 15600 | 100.00% |
| NC_003635.1 | Modoc virus, complete genome″ | 10600 | 10600 | 100.00% |
| NC_003676.1 | Apoi virus, genome″ | 10116 | 10116 | 100.00% |
| NC_003675.1 | Rio Bravo virus, genome″ | 10140 | 10140 | 100.00% |
| NC_003687.1 | Powassan virus, complete genome″ | 10839 | 10839 | 100.00% |
| NC_003690.1 | Langat virus, complete genome″ | 10943 | 10943 | 100.00% |
| NC_003790.1 | Chicken astrovirus, complete genome″ | 6927 | 6927 | 100.00% |
| NC_003899.1 | Eastern equine encephalitis virus, complete genome″ | 11675 | 11675 | 100.00% |
| NC_003908.1 | Western equine encephalomyelitis virus, complete | 11484 | 11484 | 100.00% |
| | genome″ | | | |
| NC_003977.1 | Hepatitis B virus, complete genome″ | 3215 | 3215 | 100.00% |
| NC_003990.1 | Avian encephalomyelitis virus, complete genome″ | 7055 | 7055 | 100.00% |
| NC_003996.1 | Tamana bat virus, genome″ | 10053 | 10053 | 100.00% |
| NC_004074.1 | Tioman virus, complete genome″ | 15522 | 15522 | 100.00% |
| NC_004102.1 | Hepatitis C virus genotype 1, complete genome″ | 9646 | 9646 | 100.00% |
| NC_004104.1 | Human papillomavirus type 90, complete genome″ | 8033 | 8033 | 100.00% |
| NC_004119.1 | Montana myotis leukoencephalitis virus, complete | 10690 | 10690 | 100.00% |
| | genome″ | | | |
| NC_004158.1 | Dugbe virus segment M, complete sequence″ | 4888 | 18859 | 25.92% |
| NC_004161.1 | Reston ebolavirus isolate Reston | 18891 | 18891 | 100.00% |
| | virus/M. fascicularis-tc/USA/1989/Philipp | | | |
| NC_004294.1 | Lymphocytic choriomeningitis virus segment | 3376 | 10056 | 33.57% |
| | S, complete sequence″ | | | |
| NC_004293.1 | Tacaribe virus segment S, complete sequence″ | 3432 | 10534 | 32.58% |
| NC_004295.1 | Human erythrovirus V9, complete genome″ | 5028 | 5028 | 100.00% |
| NC_004355.1 | Alkhurma virus, complete genome″ | 10685 | 10685 | 100.00% |
| NC_004451.1 | Simian sapelovirus 1, complete genome″ | 8126 | 8126 | 100.00% |
| NC_004455.1 | Simian immunodeficiency virus SIV-mnd 2, complete | 9518 | 9518 | 100.00% |
| | genome″ | | | |
| NC_004500.1 | Human papillomavirus type 92, complete genome″ | 7461 | 7461 | 100.00% |
| NC_004162.2 | Chikungunya virus, complete genome″ | 11826 | 11826 | 100.00% |
| NC_001505.2 | Murine pneumotropic virus, complete genome″ | 4754 | 4754 | 100.00% |
| NC_002076.2 | Torque teno virus 1, complete genome″ | 3852 | 3852 | 100.00% |
| NC_004713.1 | LuIII virus, complete genome″ | 5135 | 5135 | 100.00% |
| NC_004718.3 | SARS coronavirus, complete genome″ | 29751 | 29751 | 100.00% |
| NC_004800.1 | Goose hemorrhagic polyomavirus, complete genome″ | 5256 | 5256 | 100.00% |
| NC_005036.1 | Goose paramyxovirus SF02, complete genome″ | 15192 | 15192 | 100.00% |
| NC_005039.1 | Yokose virus, complete genome″ | 10857 | 10857 | 100.00% |
| NC_005062.1 | Omsk hemorrhagic fever virus, complete genome″ | 10787 | 10787 | 100.00% |
| NC_005064.1 | Kamiti River virus, complete genome″ | 11375 | 11375 | 100.00% |
| NC_005081.1 | Junin virus segment S, complete genome″ | 3411 | 10525 | 32.41% |
| NC_005078.1 | Machupo virus segment S, complete genome″ | 3439 | 10635 | 32.34% |
| NC_005077.1 | Guanarito virus segment S, complete genome″ | 3343 | 10424 | 32.07% |
| NC_005147.1 | Human coronavirus OC43, complete genome″ | 30738 | 30738 | 100.00% |
| NC_005219.1 | Hantaan virus, complete genome″ | 3616 | 11845 | 30.53% |
| NC_005234.1 | Dobrava virus segment M, complete sequence″ | 3635 | 11840 | 30.70% |
| NC_005237.1 | Seoul virus segment M, complete sequence″ | 3651 | 11950 | 30.55% |
| NC_005283.1 | Dolphin morbillivirus, complete genome″ | 15702 | 15702 | 100.00% |
| NC_005339.1 | Mossman virus, complete genome″ | 16650 | 16650 | 100.00% |
| NC_005084.2 | Fer-de-lance virus, complete genome″ | 15378 | 15378 | 100.00% |
| AY528864.1 | Macaca fuscata rhadinovirus, complete genome″ | 130497 | 131217 | 99.45% |
| NC_004148.2 | Human metapneumovirus, complete genome″ | 13335 | 13335 | 100.00% |
| NC_010624.1 | Sapovirus Mc10, complete genome″ | 7458 | 7458 | 100.00% |
| NC_005831.2 | Human coronavirus NL63, complete genome″ | 27553 | 27553 | 100.00% |
| NC_005134.2 | Human papillomavirus type 96, complete genome″ | 7438 | 7438 | 100.00% |
| NC_006144.1 | Simian adenovirus 3, complete genome″ | 34246 | 34246 | 100.00% |
| NC_006152.1 | Adeno-associated virus 5, complete genome″ | 4642 | 4642 | 100.00% |
| NC_001716.2 | Human herpesvirus 7, complete genome″ | 143280 | 152640 | 93.87% |
| NC_006260.1 | Adeno-associated virus - 7, complete genome″ | 4721 | 4721 | 100.00% |
| NC_006261.1 | Adeno-associated virus - 8, complete genome″ | 4393 | 4393 | 100.00% |
| NC_006269.1 | Sapovirus Hu/Dresden/pJG-Sap01/DE, complete | 7429 | 7429 | 100.00% |
| | genome″ | | | |
| NC_006320.1 | Toscana virus segment M, complete sequence″ | 4215 | 12488 | 33.75% |
| NC_006308.1 | Influenza C virus (C/Ann Arbor/1/50) segment | 2265 | 12555 | 18.04% |
| | 2, complete sequence″ | | | |
| NC_006447.1 | Pichinde virus, complete genome″ | 3419 | 10416 | 32.82% |
| NC_006429.1 | Mokola virus, complete genome″ | 11940 | 11940 | 100.00% |
| NC_006432.1 | Sudan ebolavirus isolate Sudan | 18875 | 18875 | 100.00% |
| | virus/H. sapiens-tc/UGA/2000/Gulu-80889 | | | |
| NC_006428.1 | Simian virus 41, complete genome″ | 15450 | 15450 | 100.00% |
| NC_006430.1 | Parainfluenza virus 5, complete genome″ | 15246 | 15246 | 100.00% |
| NC_006296.2 | Rinderpest virus (strain Kabete O), complete genome″ | 15882 | 15882 | 100.00% |
| NC_006558.1 | Getah virus, complete genome″ | 11597 | 11597 | 100.00% |
| NC_006551.1 | Usutu virus, complete genome″ | 11066 | 11066 | 100.00% |
| NC_006554.1 | Sapovirus C12 strain C12 | 7356 | 7476 | 98.39% |
| NC_006573.1 | Mopeia Lassa reassortant 29 segment S, complete | 3402 | 10673 | 31.87% |
| | genome″ | | | |
| NC_006575.1 | Mopeia virus AN20410 segment S, complete genome″ | 3427 | 10698 | 32.03% |
| NC_006579.1 | Pneumonia virus of mice J3666, complete genome″ | 14885 | 14885 | 100.00% |
| NC_006879.1 | Simian adenovirus 1, complete genome″ | 34450 | 34450 | 100.00% |
| NC_006947.1 | Karshi virus, complete genome″ | 10653 | 10653 | 100.00% |
| NC_006998.1 | Vaccinia virus, complete genome″ | 194711 | 194711 | 100.00% |
| NC_007013.1 | Small anellovirus 1, complete genome″ | 2249 | 2249 | 100.00% |
| NC_007014.1 | Small anellovirus 2, complete genome″ | 2635 | 2635 | 100.00% |
| NC_007018.1 | Human parvovirus 4 G1, complete genome″ | 5268 | 5268 | 100.00% |
| NC_006383.2 | Peste-des-petits-ruminants virus, complete genome″ | 15948 | 15948 | 100.00% |
| NC_007360.1 | Influenza A virus (A/Goose/Guangdong/1/96(H5N1)) | 1565 | 13590 | 11.52% |
| | segment 5, complete | | | |
| NC_007454.1 | J-virus, complete genome″ | 18954 | 18954 | 100.00% |
| NC_007455.1 | Human bocavirus, complete genome″ | 5299 | 5299 | 100.00% |
| NC_007605.1 | Human herpesvirus 4 complete wild type genome | 170743 | 171823 | 99.37% |
| NC_007611.1 | Simian virus 12, complete genome″ | 5230 | 5230 | 100.00% |
| NC_007620.1 | Menangle virus, complete genome″ | 15516 | 15516 | 100.00% |
| NC_007652.1 | Avian metapneumovirus | 14071 | 14071 | 100.00% |
| NC_006577.2 | Human coronavirus HKU1, complete genome″ | 29926 | 29926 | 100.00% |
| NC_007732.1 | Porcine hemagglutinating encephalomyelitis | 30480 | 30480 | 100.00% |
| | virus, complete genome″ | | | |
| NC_007803.1 | Beilong virus, complete genome″ | 19212 | 19212 | 100.00% |
| NC_007922.1 | Crow polyomavirus, complete genome″ | 5079 | 5079 | 100.00% |
| NC_007923.1 | Finch polyomavirus, complete genome″ | 5278 | 5278 | 100.00% |
| NC_001959.2 | Norwalk virus, complete genome″ | 7654 | 7654 | 100.00% |
| NC_008188.1 | Human papillomavirus type 103, complete genome″ | 7263 | 7263 | 100.00% |
| NC_008189.1 | Human papillomavirus type 101, complete genome″ | 7259 | 7259 | 100.00% |
| NC_001401.2 | Adeno-associated virus - 2, complete genome″ | 4679 | 4679 | 100.00% |
| NC_008315.1 | Bat coronavirus (BtCoV/133/2005), complete | 30307 | 30307 | 100.00% |
| | genome″ | | | |
| NC_008719.1 | Sepik virus, complete genome″ | 10793 | 10793 | 100.00% |
| NC_008718.1 | Entebbe bat virus, complete genome″ | 10510 | 10510 | 100.00% |
| NC_008724.1 | Acanthocystis turfacea Chlorella virus 1, complete | 287520 | 288000 | 99.83% |
| | genome″ | | | |
| NC_007580.2 | St. Louis encephalitis virus, complete genome″ | 10820 | 10940 | 98.90% |
| NC_009020.1 | Bat coronavirus HKU5-1, complete genome″ | 30362 | 30482 | 99.61% |
| NC_009225.1 | Torque teno midi virus 1, complete genome″ | 3245 | 3245 | 100.00% |
| NC_009238.1 | KI polyomavirus Stockholm 60, complete genome″ | 5040 | 5040 | 100.00% |
| NC_009333.1 | Human herpesvirus 8, complete genome″ | 137369 | 137969 | 99.57% |
| NC_003976.2 | Ljungan virus, complete genome″ | 7590 | 7590 | 100.00% |
| NC_009489.1 | Mapuera virus, complete genome″ | 15486 | 15486 | 100.00% |
| NC_009527.1 | European bat lyssavirus 1, complete genome″ | 11966 | 11966 | 100.00% |
| NC_009528.1 | European bat lyssavirus 2, complete genome″ | 11930 | 11930 | 100.00% |
| NC_009539.1 | WU Polyomavirus, complete genome″ | 5229 | 5229 | 100.00% |
| NC_009640.1 | Porcine rubulavirus, complete genome″ | 15180 | 15180 | 100.00% |
| NC_009657.1 | Scotophilus bat coronavirus 512, complete genome″ | 28203 | 28203 | 100.00% |
| NC_009825.1 | Hepatitis C virus genotype 4, genome″ | 9235 | 9355 | 98.72% |
| NC_009826.1 | Hepatitis C virus genotype 5, genome″ | 9343 | 9343 | 100.00% |
| NC_009823.1 | Hepatitis C virus genotype 2, complete genome″ | 9711 | 9711 | 100.00% |
| NC_009827.1 | Hepatitis C virus genotype 6, complete genome″ | 9628 | 9628 | 100.00% |
| NC_009824.1 | Hepatitis C virus genotype 3, genome″ | 9456 | 9456 | 100.00% |
| NC_001608.3 | Marburg marburgvirus isolate Marburg | 19111 | 19111 | 100.00% |
| | virus/H. sapiens-tc/KEN/1980/Mt. | | | |
| NC_001474.2 | Dengue virus 2, complete genome″ | 10723 | 10723 | 100.00% |
| NC_009951.1 | Squirrel monkey polyomavirus, complete genome″ | 5075 | 5075 | 100.00% |
| NC_009988.1 | Bat coronavirus HKU2, complete genome″ | 27165 | 27165 | 100.00% |
| NC_001475.2 | Dengue virus 3, complete genome″ | 10707 | 10707 | 100.00% |
| NC_010248.1 | Oliveros virus segment S, complete sequence″ | 3535 | 10719 | 32.98% |
| NC_010247.1 | Amapari virus segment S, complete sequence″ | 3341 | 10387 | 32.17% |
| NC_008604.2 | Culex flavivirus, complete genome″ | 10837 | 10837 | 100.00% |
| NC_010329.1 | Human papillomavirus type 88, complete genome″ | 7326 | 7326 | 100.00% |
| NC_010436.1 | Bat coronavirus 1B, complete genome″ | 28476 | 28476 | 100.00% |
| NC_010437.1 | Bat coronavirus 1A, complete genome″ | 28326 | 28326 | 100.00% |
| NC_010438.1 | Bat coronavirus HKU8, complete genome″ | 28773 | 28773 | 100.00% |
| NC_009448.2 | Saffold virus, complete genome″ | 8115 | 8115 | 100.00% |
| NC_010702.1 | Tamiami virus segment L, complete sequence″ | 7141 | 10674 | 66.90% |
| NC_010757.1 | Flexal virus segment S, complete sequence″ | 3410 | 10448 | 32.64% |
| NC_010758.1 | Latino virus segment S, complete sequence″ | 3474 | 10648 | 32.63% |
| NC_010746.1 | Mycoreovirus 1 segment 4, complete sequence″ | 2269 | 23433 | 9.68% |
| NC_010810.1 | Human TMEV-like cardiovirus, complete genome″ | 7961 | 7961 | 100.00% |
| NC_010820.1 | Simian foamy virus 3, complete genome″ | 13111 | 13111 | 100.00% |
| NC_010819.1 | Macaque simian foamy virus, complete genome″ | 12972 | 12972 | 100.00% |
| NC_010956.1 | Human adenovirus D, complete genome″ | 35083 | 35083 | 100.00% |
| NC_011203.1 | Human adenovirus B1, complete genome″ | 35343 | 35343 | 100.00% |
| NC_011310.1 | Myotis polyomavirus VM-2008, complete genome″ | 5081 | 5081 | 100.00% |
| NC_011400.1 | Astrovirus MLB1, complete genome″ | 6171 | 6171 | 100.00% |
| NC_011546.1 | Simian T-cell lymphotropic virus 6, complete | 8913 | 8913 | 100.00% |
| | genome″ | | | |
| NC_011829.1 | Porcine kobuvirus | 8210 | 8210 | 100.00% |
| | swine/S-1-HUN/2007/Hungary, complete genome″ | | | |
| NC_011800.1 | Human T-lymphotropic virus 4, complete genome″ | 8791 | 8791 | 100.00% |
| NC_012042.1 | Humanbocavirus 2c PK isolate PK-5510, complete | 5076 | 5196 | 97.69% |
| | genome″ | | | |
| NC_001664.2 | Human herpesvirus 6A, complete genome″ | 157200 | 158880 | 98.94% |
| NC_012126.1 | California sea lion anellovirus, complete genome″ | 2140 | 2140 | 100.00% |
| NC_012213.1 | Human papillomavirus type 108, complete genome″ | 7149 | 7149 | 100.00% |
| NC_012437.1 | Duck astrovirus C-NGB, complete genome″ | 7722 | 7722 | 100.00% |
| NC_012485.1 | Human papillomavirus type 109, complete genome″ | 7346 | 7346 | 100.00% |
| NC_012486.1 | Human papillomavirus type 112, complete genome″ | 7227 | 7227 | 100.00% |
| NC_012532.1 | Zika virus, complete genome″ | 10794 | 10794 | 100.00% |
| NC_012533.1 | Kedougou virus, complete genome″ | 10723 | 10723 | 100.00% |
| NC_012534.1 | Bagaza virus, complete genome″ | 10941 | 10941 | 100.00% |
| NC_012564.1 | Human bocavirus 3, complete genome″ | 5242 | 5242 | 100.00% |
| NC_012671.1 | Quang Binh virus, complete genome″ | 10865 | 10865 | 100.00% |
| NC_012735.1 | Wesselsbron virus, complete genome″ | 10814 | 10814 | 100.00% |
| NC_012776.1 | Lujo virus segment S, complete genome″ | 3189 | 10352 | 30.81% |
| NC_012798.1 | Human cosavirus E1, complete genome″ | 6580 | 6580 | 100.00% |
| NC_012801.1 | Human cosavirus B1, complete genome″ | 7085 | 7205 | 98.33% |
| NC_012802.1 | Human cosavirus D1, complete genome″ | 7215 | 7215 | 100.00% |
| NC_012950.1 | Human enteric coronavirus strain 4408, complete | 25553 | 30953 | 82.55% |
| | genome″ | | | |
| NC_012959.1 | Human adenovirus 54, complete genome″ | 34920 | 34920 | 100.00% |
| NC_012932.1 | Aedes flavivirus genomic RNA, complete genome | 11064 | 11064 | 100.00% |
| NC_013035.1 | Human papillomavirus 116, complete genome″ | 7184 | 7184 | 100.00% |
| NC_013057.1 | Morogoro virus segment S, complete genome″ | 3383 | 10590 | 31.95% |
| NC_009026.2 | Bussuquara virus, complete genome″ | 10815 | 10815 | 100.00% |
| NC_013439.1 | Orangutan polyomavirus, complete genome″ | 5168 | 5168 | 100.00% |
| NC_013796.1 | California sea lion polyomavirus 1, complete genome″ | 5112 | 5112 | 100.00% |
| NC_012729.2 | Human bocavirus 4 NI strain | 5104 | 5104 | 100.00% |
| | HBoV4-NI-385, complete genome″ | | | |
| NC_009029.2 | Kokobera virus, complete genome″ | 10874 | 10874 | 100.00% |
| NC_009028.2 | Ilheus virus, complete genome″ | 10755 | 10755 | 100.00% |
| NC_014072.1 | Torque teno felis virus, complete genome″ | 2064 | 2064 | 100.00% |
| NC_014073.1 | Torque teno virus 28, complete genome″ | 3629 | 3629 | 100.00% |
| NC_014068.1 | Torque teno mini virus 8, complete genome″ | 2910 | 2910 | 100.00% |
| NC_014074.1 | Torque teno virus 27, complete genome″ | 3729 | 3729 | 100.00% |
| NC_014075.1 | Torque teno virus 12, complete genome″ | 3759 | 3759 | 100.00% |
| NC_014076.1 | Torque teno virus 10, complete genome″ | 3770 | 3770 | 100.00% |
| NC_014069.1 | Torque teno virus 4, complete genome″ | 3690 | 3690 | 100.00% |
| NC_014070.1 | Torque teno sus virus 1, complete genome″ | 2878 | 2878 | 100.00% |
| NC_014071.1 | Torque teno canis virus, complete genome″ | 2797 | 2797 | 100.00% |
| NC_014077.1 | Torque teno virus 14, complete genome″ | 3899 | 3899 | 100.00% |
| NC_014078.1 | Torque teno virus 19, complete genome″ | 3208 | 3808 | 84.24% |
| NC_014079.1 | Torque teno virus 26, complete genome″ | 3798 | 3798 | 100.00% |
| NC_014080.1 | Torque teno virus 7, complete genome″ | 3736 | 3736 | 100.00% |
| NC_014081.1 | Torque teno virus 3, complete genome″ | 3748 | 3748 | 100.00% |
| NC_014082.1 | Torque teno mini virus 7, complete genome″ | 2760 | 2880 | 95.83% |
| NC_014083.1 | Torque teno virus 25, complete genome″ | 3763 | 3763 | 100.00% |
| NC_014084.1 | Torque teno virus 8, complete genome″ | 3790 | 3790 | 100.00% |
| NC_014085.1 | Torque teno tamarin virus, complete genome″ | 3371 | 3371 | 100.00% |
| NC_014086.1 | Torque teno mini virus 2, complete genome″ | 2640 | 2640 | 100.00% |
| NC_014087.1 | Torque teno douroucouli virus, complete genome″ | 3718 | 3718 | 100.00% |
| NC_014088.1 | Torque teno mini virus 3, complete genome″ | 2760 | 2760 | 100.00% |
| NC_014089.1 | Torque teno mini virus 5, complete genome″ | 2908 | 2908 | 100.00% |
| NC_014090.1 | Torque teno mini virus 4, complete genome″ | 2785 | 2785 | 100.00% |
| NC_014091.1 | Torque teno virus 16, complete genome″ | 3818 | 3818 | 100.00% |
| NC_014093.1 | Torque teno midi virus 2, complete genome″ | 3253 | 3253 | 100.00% |
| NC_014094.1 | Torque teno virus 6, complete genome″ | 3705 | 3705 | 100.00% |
| NC_014095.1 | Torque teno mini virus 6, complete genome″ | 2897 | 2897 | 100.00% |
| NC_014096.1 | Torque teno virus 15, complete genome″ | 3787 | 3787 | 100.00% |
| NC_014097.1 | Torque teno mini virus 1, complete genome″ | 2856 | 2856 | 100.00% |
| NC_014185.1 | Human papillomavirus 121, complete genome″ | 7342 | 7342 | 100.00% |
| NC_014372.1 | Tai Forest ebolavirus isolate Tai Forest | 18935 | 18935 | 100.00% |
| | virus/H. sapiens-tc/CIV/1994/Paule | | | |
| NC_014361.1 | Trichodysplasia spinulosa-associated | 5232 | 5232 | 100.00% |
| | polyomavirus, complete genome″ | | | |
| NC_014373.1 | Bundibugyo ebolavirus isolate Bundibugyo | 18940 | 18940 | 100.00% |
| | virus/H. sapiens-tc/UGA/2007/ | | | |
| NC_009424.4 | Woolly monkey sarcoma virus, complete genome″ | 4079 | 5159 | 79.07% |
| NC_014407.1 | Human polyomavirus 7, complete genome″ | 4952 | 4952 | 100.00% |
| NC_014406.1 | Human polyomavirus 6, complete genome″ | 4926 | 4926 | 100.00% |
| NC_014468.1 | Bat adeno-associated virus YNM, complete genome″ | 4286 | 4286 | 100.00% |
| NC_014469.1 | Gammapapillomavirus HPV127, complete genome″ | 7181 | 7181 | 100.00% |
| NC_014470.1 | Bat coronavirus BM48-31/BGR/2008, complete | 29276 | 29276 | 100.00% |
| | genome″ | | | |
| NC_014474.1 | Simian retrovirus 4, complete genome″ | 8126 | 8126 | 100.00% |
| NC_001526.2 | Human papillomavirus type 16, complete genome″ | 7905 | 7905 | 100.00% |
| NC_014480.2 | Torque teno virus 2, complete genome″ | 3322 | 3322 | 100.00% |
| NC_014092.2 | Torque teno sus virus k2 isolate 2p, complete | 2520 | 2520 | 100.00% |
| | genome″ | | | |
| NC_014743.1 | Chimpanzee polyomavirus, complete genome″ | 5086 | 5086 | 100.00% |
| NC_004764.2 | Budgerigar fledgling disease virus - 1, complete | 4981 | 4981 | 100.00% |
| | genome″ | | | |
| NC_014929.1 | Cyclovirus bat/USA/2009, complete genome″ | 1703 | 1703 | 100.00% |
| NC_014956.1 | Human papillomavirus type 134, complete genome″ | 7309 | 7309 | 100.00% |
| NC_014955.1 | Human papillomavirus type 132, complete genome″ | 7125 | 7125 | 100.00% |
| NC_014954.1 | Human papillomavirus type 131, complete genome″ | 7182 | 7182 | 100.00% |
| NC_014953.1 | Human papillomavirus type 129, complete genome″ | 7219 | 7219 | 100.00% |
| NC_014952.1 | Human papillomavirus type 128, complete genome″ | 7259 | 7259 | 100.00% |
| NC_015212.1 | Seal anellovirus TFFN/USA/2006, complete genome″ | 2164 | 2164 | 100.00% |
| NC_015150.1 | Human polyomavirus 9, complete genome″ | 5026 | 5026 | 100.00% |
| NC_015225.1 | Simian adenovirus 49, complete genome″ | 35499 | 35499 | 100.00% |
| NC_015521.1 | Cutthroat trout virus, complete genome″ | 7310 | 7310 | 100.00% |
| NC_001545.2 | Rubella virus, complete genome″ | 9762 | 9762 | 100.00% |
| NC_015691.1 | Macaca fascicularis papillomavirus type 2, complete | 7632 | 7632 | 100.00% |
| | genome″ | | | |
| NC_015692.1 | Colobus guereza papillomavirus type 2, complete | 7686 | 7686 | 100.00% |
| | genome″ | | | |
| NC_015783.1 | Torque teno virus, complete genome″ | 3605 | 3725 | 96.78% |
| NC_015934.1 | Bat picomavirus 3, complete genome″ | 7749 | 7749 | 100.00% |
| NC_015940.1 | Bat picomavirus 1, complete genome″ | 7753 | 7753 | 100.00% |
| NC_015941.1 | Bat picomavirus 2, complete genome″ | 7693 | 7693 | 100.00% |
| NC_015932.1 | Bat adenovirus 2, complete genome″ | 31616 | 31616 | 100.00% |
| NC_015935.1 | Mouse astrovirus M-52/USA/2008, complete | 6543 | 6543 | 100.00% |
| | genome″ | | | |
| NC_016144.1 | Lloviu cuevavirus isolate Lloviu | 18927 | 18927 | 100.00% |
| | virus/M. schreibersii-wt/ESP/2003/Asturias | | | |
| NC_000883.2 | Human parvovirus B19, complete genome″ | 5596 | 5596 | 100.00% |
| NC_016154.1 | Cynomolgus macaque cytomegalovirus strain | 218041 | 218041 | 100.00% |
| | Ottawa, complete genome″ | | | |
| NC_016157.1 | Human papillomavirus type 126, complete genome″ | 7326 | 7326 | 100.00% |
| NC_016744.1 | Eidolon helvum parvovirus 1, complete genome″ | 4945 | 5065 | 97.63% |
| NC_016895.1 | Bat adenovirus TJM, complete genome″ | 31681 | 31681 | 100.00% |
| NC_016896.1 | Astrovirus wild | 6707 | 6707 | 100.00% |
| | boar/WBAstV-1/2011/HUN, complete genome″ | | | |
| NC_016155.1 | Astrovirus MLB2, complete genome″ | 6119 | 6119 | 100.00% |
| NC_016997.1 | Donggang virus, complete genome″ | 10791 | 10791 | 100.00% |
| NC_015843.2 | Tembusu virus strain JS804, complete genome″ | 10990 | 10990 | 100.00% |
| NC_017936.1 | Bat sapovirus TLC58/HK, complete genome″ | 7696 | 7696 | 100.00% |
| NC_017937.1 | Nariva virus, complete genome″ | 15276 | 15276 | 100.00% |
| NC_017982.1 | Equine polyomavirus, complete genome″ | 4987 | 4987 | 100.00% |
| NC_017993.1 | Human papillomavirus type 135, complete genome″ | 7293 | 7293 | 100.00% |
| NC_017994.1 | Human papillomavirus type 136, complete genome″ | 7319 | 7319 | 100.00% |
| NC_017995.1 | Human papillomavirus type 137, complete genome″ | 7236 | 7236 | 100.00% |
| NC_017996.1 | Human papillomavirus type 140, complete genome″ | 7341 | 7341 | 100.00% |
| NC_017997.1 | Human papillomavirus type 144, complete genome″ | 7271 | 7271 | 100.00% |
| NC_017086.1 | Chaoyang virus, complete genome″ | 10733 | 10733 | 100.00% |
| NC_017085.1 | Canary polyomavirus, complete genome″ | 5421 | 5421 | 100.00% |
| JX262162.1 | Human polyomavirus 10 isolate 10 ww, complete | 4939 | 4939 | 100.00% |
| | genome″ | | | |
| NC_018382.1 | Bat hepevirus, complete genome″ | 6796 | 6796 | 100.00% |
| NC_018481.1 | CAS virus segment S, complete genome″ | 3368 | 10180 | 33.08% |
| NC_018482.1 | Golden Gate virus segment L, complete genome″ | 6922 | 10404 | 66.53% |
| NC_018629.1 | Ikoma lyssavirus, complete genome″ | 11902 | 11902 | 100.00% |
| NC_018669.1 | Astrovirus VA2, complete genome″ | 6531 | 6531 | 100.00% |
| NC_018702.1 | Murine astrovirus, complete genome″ | 6838 | 6838 | 100.00% |
| NC_018871.1 | Rousettus bat coronavirus HKU10, complete genome″ | 28494 | 28494 | 100.00% |
| NC_019023.1 | Human papillomavirus type 166 isolate KC9, complete | 7212 | 7212 | 100.00% |
| | genome″ | | | |
| NC_019026.1 | Astrovirus VA3 isolate | 6581 | 6581 | 100.00% |
| | VA3/human/Vellore/28054/2005, complete genome | | | |
| NC_019027.1 | Astrovirus VA4 isolate | 6518 | 6518 | 100.00% |
| | VA4/human/Nepal/s5363, complete genome″ | | | |
| NC_019028.1 | Astrovirus MLB3 isolate | 6124 | 6124 | 100.00% |
| | MLB3/human/Vellore/26564/2004, complete geno | | | |
| NC_019494.1 | Porcine astrovirus 3 isolate US-MO123, complete | 6460 | 6460 | 100.00% |
| | genome″ | | | |
| NC_019531.1 | Avian paramyxovirus 4 strain | 15048 | 15048 | 100.00% |
| | APMV-4/duck/Delaware/549227/2010, comp | | | |
| NC_019844.1 | Vervet monkey polyomavirus 1 DNA, complete | 5157 | 5157 | 100.00% |
| | genome | | | |
| NC_019850.1 | Piliocolobus rufomitratus polyomavirus 1, complete | 5140 | 5140 | 100.00% |
| | genome″ | | | |
| NC_019851.1 | Macaca fascicularis polyomavirus 1, complete | 5087 | 5087 | 100.00% |
| | genome″ | | | |
| NC_019853.1 | Ateles paniscus polyomavirus 1, complete genome″ | 5273 | 5273 | 100.00% |
| NC_019855.1 | Pan troglodytes verus polyomavirus 3, complete | 5333 | 5333 | 100.00% |
| | genome″ | | | |
| NC_019856.1 | Pan troglodytes verus polyomavirus 4, complete | 5349 | 5349 | 100.00% |
| | genome″ | | | |
| NC_019857.1 | Pan troglodytes verus polyomavirus 5, complete | 4994 | 4994 | 100.00% |
| | genome″ | | | |
| NC_019858.1 | Pan troglodytes schweinfurthii polyomavirus | 4970 | 4970 | 100.00% |
| | 2, complete genome″ | | | |
| NC_020065.1 | Chaerephon polyomavirus 1 isolate KY397, complete | 4899 | 4899 | 100.00% |
| | genome″ | | | |
| NC_020066.1 | Otomops polyomavirus 2 isolate KY156, complete | 4914 | 4914 | 100.00% |
| | genome″ | | | |
| NC_020067.1 | Cardioderma polyomavirus isolate KY336, complete | 5372 | 5372 | 100.00% |
| | genome″ | | | |
| NC_020068.1 | Eidolon polyomavirus 1 isolate KY270, complete | 5294 | 5294 | 100.00% |
| | genome″ | | | |
| NC_020069.1 | Miniopterus polyomavirus isolate KY369, complete | 5213 | 5213 | 100.00% |
| | genome″ | | | |
| NC_020070.1 | Pteronotus polyomavirus isolate GTM203, complete | 5136 | 5136 | 100.00% |
| | genome″ | | | |
| NC_020071.1 | Otomops polyomavirus 1 isolate KY157, complete | 5176 | 5176 | 100.00% |
| | genome″ | | | |
| JX463184.1 | STL polyomavirus strain WD972, complete genome″ | 4775 | 4775 | 100.00% |
| NC_020106.1 | STL polyomavirus strain MA138, complete genome″ | 4776 | 4776 | 100.00% |
| NC_015630.1 | Human gyrovirus type 1, complete genome″ | 2315 | 2315 | 100.00% |
| NC_020485.1 | Simian adenovirus 20 strain ATCC VR-541, complete | 34302 | 34302 | 100.00% |
| | genome″ | | | |
| NC_020498.1 | TTV-like mini virus isolate TTMV_LY1, complete | 2912 | 2912 | 100.00% |
| | genome″ | | | |
| NC_020805.1 | Chandipura virus isolate CIN 0451, complete genome″ | 11120 | 11120 | 100.00% |
| NC_020807.1 | Lagos bat virus isolate 0406SEN, complete genome″ | 12016 | 12016 | 100.00% |
| NC_020808.1 | Aravan virus, complete genome″ | 11918 | 11918 | 100.00% |
| NC_020809.1 | Irkut virus, complete genome″ | 11980 | 11980 | 100.00% |
| NC_020810.1 | Duvenhage virus isolate 86132SA, complete genome″ | 11856 | 11976 | 99.00% |
| NC_020881.1 | Bat hepatitis virus isolate 776, complete genome″ | 3230 | 3230 | 100.00% |
| NC_020890.1 | Human polyomavirus 12 strain hu1403, complete | 5033 | 5033 | 100.00% |
| | genome″ | | | |
| NC_021069.1 | Mosquito flavivirus isolate | 10385 | 10865 | 95.58% |
| | LSFlaviV-A20-09, complete genome″ | | | |
| NC_021153.1 | Rodent hepacivirus isolate RHV-339, complete | 8879 | 8879 | 100.00% |
| | genome″ | | | |
| NC_021168.1 | Simian adenovirus C isolate BaAdV-2, complete | 34391 | 34391 | 100.00% |
| | genome″ | | | |
| NC_020487.1 | Titi monkey adenovirus ECC-2011, complete genome″ | 36838 | 36838 | 100.00% |
| NC_021206.1 | Bat circovirus isolate XOR7, complete genome″ | 1798 | 1798 | 100.00% |
| NC_021483.1 | Human papillomavirus type 154 isolate | 7286 | 7286 | 100.00% |
| | PV77, complete genome″ | | | |
| NC_021568.1 | Human cyclovirus VS5700009, complete genome″ | 1832 | 1832 | 100.00% |
| NC_021928.1 | Human parainfluenza vims 4a viral cRNA, complete | 17052 | 17052 | 100.00% |
| | genome | | | |
| NC_001906.3 | Hendra virus, complete genome″ | 18234 | 18234 | 100.00% |
| NC_022095.1 | Human papillomavirus type 179 complete | 7228 | 7228 | 100.00% |
| | genome, isolate SIBX16″ | | | |
| NC_004763.2 | African green monkey polyomavirus, complete | 5270 | 5270 | 100.00% |
| | genome″ | | | |
| NC_022103.1 | Bat coronavirus CDPHE15/USA/2006, complete | 28035 | 28035 | 100.00% |
| | genome″ | | | |
| NC_022249.1 | Feline astrovirus 2 strain 1637F, complete genome″ | 6795 | 6795 | 100.00% |
| NC_022266.1 | Simian adenovirus 18, complete genome″ | 31967 | 31967 | 100.00% |
| NC_019854.2 | Cebus albifrons polyomavirus 1, complete genome″ | 5013 | 5013 | 100.00% |
| NC_022519.1 | African elephant polyomavirus 1, complete genome″ | 5722 | 5722 | 100.00% |
| NC_022631.1 | Razdan virus strain LEIV-Arm2741 segment | 3303 | 11470 | 28.80% |
| | M, complete sequence″ | | | |
| NC_022755.1 | American bat vesiculovirus TFFN-2013 isolate | 10212 | 10692 | 95.51% |
| | liver2008, complete genome | | | |
| NC_022892.1 | Human papillomavirus type 167 isolate | 7228 | 7228 | 100.00% |
| | KC10, complete genome″ | | | |
| NC_018705.3 | Ntaya virus isolate IPDIA, complete genome″ | 10943 | 10943 | 100.00% |
| NC_023008.1 | Butcherbird polyomavirus isolate | 5084 | 5084 | 100.00% |
| | AWH19840, complete genome″ | | | |
| NC_023629.1 | Bovine astrovirus B76/HK, complete genome″ | 6253 | 6253 | 100.00% |
| NC_023630.1 | Bovine astrovirus B76-2/HK, complete genome″ | 6301 | 6301 | 100.00% |
| NC_023631.1 | Bovine astrovirus B18/HK, complete genome″ | 6300 | 6300 | 100.00% |
| NC_023632.1 | Bovine astrovirus B170/HK, complete genome″ | 6317 | 6317 | 100.00% |
| NC_023635.1 | Arumowot virus segment L, complete sequence″ | 6376 | 12262 | 52% |
| NC_023636.1 | Porcine astrovirus 5 isolate | 6500 | 6500 | 100.00% |
| | AstV5-US-IA122, complete genome″ | | | |
| NC_023674.1 | Porcine astrovirus 2 strain 43/USA, complete genome″ | 6318 | 6318 | 100.00% |
| NC_023675.1 | Porcine astrovirus 4 strain 35/USA, complete genome″ | 6480 | 6600 | 98.18% |
| NC_023874.1 | Human cyclovirus strain 7078A, complete genome″ | 1790 | 1790 | 100.00% |
| NC_023891.1 | Human papillomavirus type 178, complete genome″ | 7314 | 7314 | 100.00% |
| NC_023984.1 | Human cosavirus isolate | 7802 | 7802 | 100.00% |
| | Cosavirus_Amsterdam_1994, complete genome″ | | | |
| KF954417.1 | New Jersey polyomavirus-2013 isolate | 5108 | 5108 | 100.00% |
| | NJ-PyV-2013, complete genome″ | | | |
| NC_024075.1 | Cat Que Virus strain VN04-2108 nucleoprotein | 983 | 12390 | 7.93% |
| | gene, complete cds″ | | | |
| NC_024296.1 | Avian bornavirus isolate VS-4424 nucleoprotein | 8914 | 8914 | 100.00% |
| | (N), X protein (X) | | | |
| NC_024297.1 | Bovine astrovirus strain | 6233 | 6233 | 100.00% |
| | BAstV-GX7/CHN/2014, complete genome″ | | | |
| NC_024306.1 | Fruit bat alphaherpesvirus 1 DNA, complete genome″ | 149339 | 149459 | 99.92% |
| NC_024377.1 | Simian pegivirus isolate SPgVkrc_RC08 polyprotein | 9525 | 9525 | 100.00% |
| | precursor, complete cd | | | |
| NC_024443.1 | Roundleaf bat hepatitis B virus isolate | 3368 | 3368 | 100.00% |
| | RBHBV/GB09-256/Hip_rub/GAB/20 | | | |
| NC_024444.1 | Horseshoe bat hepatitis B virus isolate | 3377 | 3377 | 100.00% |
| | HBHBV/GB09-403/Rhi_alc/GAB/20 | | | |
| NC_024445.1 | Tent-making bat hepatitis B virus isolate | 3149 | 3149 | 100.00% |
| | TBHBV/Pan372/Uro_bil/PAN/201 | | | |
| NC_021707.2 | Cyclovirus VN isolate hcf1, complete genome″ | 1855 | 1855 | 100.00% |
| NC_024472.1 | Human astrovirus BF34, complete genome″ | 6577 | 6577 | 100.00% |
| NC_024496.1 | Heartland virus isolate Patient1 segment S, complete | 1772 | 11567 | 15.32% |
| | sequence″ | | | |
| NC_024498.1 | Bovine astrovirus CH13, complete genome″ | 6530 | 6530 | 100.00% |
| LK931491.1 | Sphinx1.76-related DNA, replication competent | 1766 | 1766 | 100.00% |
| | episomal DNA MSBI1.176″ | | | |
| LK931492.1 | Sphinx1.76-related DNA, replication competent | 1766 | 1766 | 100.00% |
| | episomal DNA MSBI2.176″ | | | |
| NC_024689.1 | HCBI8.215 virus complete sequence | 2032 | 2152 | 94.42% |
| NC_024690.1 | HCBI9.212 virus complete sequence | 2121 | 2121 | 100.00% |
| NC_024691.1 | MSSI2.225 virus complete sequence | 2259 | 2259 | 100.00% |
| NC_024694.1 | Human circovirus VS6600022, complete genome″ | 2836 | 2836 | 100.00% |
| NC_024701.1 | Feline astrovirus D1 isolate FAstV-D1, complete | 6598 | 6598 | 100.00% |
| | genome″ | | | |
| NC_024778.1 | Reptile bornavirus 1 strain 251327, complete genome″ | 8884 | 8884 | 100.00% |
| NC_024890.1 | Seal anellovirus 3, complete genome″ | 2169 | 2169 | 100.00% |
| NC_024891.1 | Seal anellovirus 2, complete genome″ | 2149 | 2149 | 100.00% |
| NC_024908.1 | Torque teno Tadarida brasiliensis virus, complete | 2367 | 2367 | 100.00% |
| | genome″ | | | |
| NC_025217.1 | Bat Hp-betacoronavirus/Zhejiang2013, complete | 31491 | 31491 | 100.00% |
| | genome″ | | | |
| NC_025251.1 | Bokeloh bat lyssavirus isolate 21961, complete | 11900 | 11900 | 100.00% |
| | genome″ | | | |
| NC_025256.1 | Bat Paramyxovirus | 18530 | 18530 | 100.00% |
| | Eid_hel/GH-M74a/GHA/2009, complete genome″ | | | |
| NC_025341.1 | Fikirini bat rhabdovirus isolate KEN352, complete | 11139 | 11139 | 100.00% |
| | genome″ | | | |
| NC_025346.1 | Rabbit astrovirus TN/2208/2010, complete genome″ | 7353 | 7353 | 100.00% |
| NC_025365.1 | Shimoni bat virus, complete genome″ | 12045 | 12045 | 100.00% |
| NC_025370.1 | Pan troglodytes verus polyomavirus 2a isolate | 5309 | 5309 | 100.00% |
| | 6512, complete genome″ | | | |
| NC_025377.1 | West Caucasian bat virus, complete genome″ | 12278 | 12278 | 100.00% |
| NC_025403.1 | Achimota virus 1, complete genome″ | 15624 | 15624 | 100.00% |
| NC_025404.1 | Achimota virus 2, complete genome″ | 15504 | 15504 | 100.00% |
| NC_025409.1 | Astrovirus SG, complete genome″ | 6584 | 6584 | 100.00% |
| NC_001659.2 | African swine fever virus strain BA71V, complete | 170101 | 170101 | 100.00% |
| | genome″ | | | |
| NC_003092.2 | Simian hemorrhagic fever virus, complete genome″ | 14997 | 15717 | 95.42% |
| NC_025678.1 | Simian adenovirus DM-2014 isolate 23336, complete | 32541 | 32661 | 99.63% |
| | genome″ | | | |
| NC_001515.2 | Murine polyomavirus strain BG, complete genome″ | 5307 | 5307 | 100.00% |
| NC_001663.2 | Hamster polyomavirus isolate Berlin-Buch, complete | 5372 | 5372 | 100.00% |
| | genome″ | | | |
| NC_010277.2 | Merkel cell polyomavirus isolate R17b, complete | 5387 | 5387 | 100.00% |
| | genome″ | | | |
| GQ466044.1 | Human herpesvirus 5 strain 3301, complete genome″ | 235583 | 235703 | 99.95% |
| GQ221973.1 | Human herpesvirus 5 strain HAN13, complete | 236219 | 236219 | 100.00% |
| | genome″ | | | |
| GQ221974.1 | Human herpesvirus 5 strain 3157, complete genome″ | 235154 | 235154 | 100.00% |
| KP745728.1 | Human herpesvirus 5 strain BE/4/2010, complete | 236428 | 236428 | 100.00% |
| | genome″ | | | |
| KU681082.3 | Zika virus isolate Zika | 10807 | 10807 | 100.00% |
| | virus/H. sapiens-tc/PHL/2012/CPC-0740, complete ge | | | |
| A06324.1 | HPV type18 E6/E7 | 3769 | 7906 | 47.67% |
| A09292.1 | HPV type1a L1 | 1527 | 7478 | 20.42% |
| DJ042838.1 | HPV type45 L1 | 1533 | 8039 | 19.07% |
| DQ091857.1 | HPV type97 E6/E7 | 624 | 7843 | 7.96% |
| FM876121.1 | HPV type6 L1 | 1503 | 8051 | 18.67% |
| GQ228066.1 | HPV type52 E6/E7 | 898 | 7960 | 11.28% |
| GQ288789.1 | HPV type81 L1 | 1599 | 8067 | 19.82% |
| GQ479012.1 | HPV type33 L1 | 1500 | 7909 | 18.97% |
| GQ479020.1 | HPV type35 L1 | 1509 | 7908 | 19.08% |
| GQ487711.1 | HPV type51 L1 | 1515 | 7811 | 19.40% |
| HE793049.1 | HPV type40 L1 | 1518 | 7890 | 19.24% |
| HE820129.1 | HPV type42 L1 | 1509 | 7920 | 19.05% |
| HE962401.1 | HPV type43 L1 | 1512 | 7975 | 18.96% |
| HE963119.1 | HPV type44 L1 | 1503 | 7833 | 19.19% |
| HE963156.1 | HPV type55 L1 | 1506 | 7822 | 19.25% |
| HQ724330.1 | HPV type83 E6/E7 | 713 | 8104 | 8.80% |
| JN104068.1 | HPV type39 L1 | 1518 | 7885 | 19.25% |
| JN104073.1 | HPV type59 L1 | 1527 | 7898 | 19.33% |
| JN874416.1 | HPV type52 L1 | 1590 | 7960 | 19.97% |
| JQ963486.1 | HPV type120 L1 | 1521 | 7303 | 20.83% |
| JQ976760.1 | HPV type33 E6/E7 | 1050 | 7909 | 13.28% |
| KU163569.1 | HPV type31 L1 | 1551 | 7901 | 19.63% |
| KU550602.1 | HPV type58 L1 | 1575 | 7824 | 20.13% |
| KU721789.1 | HPV type18 L1 | 1707 | 7857 | 21.73% |
| KU721801.1 | HPV type53 L1 | 1272 | 7863 | 16.18% |
| KU951267.1 | HPV type53 E6/E7 | 785 | 7863 | 9.98% |
| KX545349.1 | HPV type31 E6/E7 | 550 | 7901 | 6.96% |
| KX545350.1 | HPV type42 E6/E7 | 525 | 7920 | 6.63% |
| KX545353.1 | HPV type81 E6/E7 | 527 | 8067 | 6.53% |
| KX545356.1 | HPV type6 E6/E7 | 531 | 8051 | 6.60% |
| KX545358.1 | HPV type74 E6/E7 | 526 | 7887 | 6.67% |
| KX545361.1 | HPV type86 E6/E7 | 507 | 7983 | 6.35% |
| KX545364.1 | HPV type43 E6/E7 | 513 | 7975 | 6.43% |
| KX545366.1 | HPV type66 E6/E7 | 558 | 7824 | 7.13% |
| KX645743.1 | HPV type56 E6/E7 | 1217 | 7822 | 15.56% |
| KX645765.1 | HPV type56 L1 | 1605 | 7822 | 20.52% |
| KY348861.1 | HPV type17 L1 | 1524 | 7426 | 20.52% |
| KY565593.1 | HPV type35 E6/E7 | 1467 | 7908 | 18.55% |
| KY565603.1 | HPV type45 E6/E7 | 1449 | 7858 | 18.44% |
| KY565665.1 | HPV type58 E6/E7 | 1447 | 7824 | 18.49% |
| L36108.1 | HPV type 11 L1/E6/E7 | 1080 | 7949 | 13.59% |
| MF588762.1 | HPV type8 L1 | 1752 | 7298 | 24.01% |
| MF588765.1 | HPV type11 L1 | 1863 | 7949 | 23.44% |
| MF588778.1 | HPV type19 L1 | 1670 | 7289 | 22.91% |
| MF588781.1 | HPV type19 E6/E7 | 2056 | 7289 | 28.21% |
| MG921179.1 | HPV type198 L1 | 1533 | 7900 | 19.41% |
| NC_001926.1 | Bunyamwera virus segment M | 4458 | 12300 | 36.24% |
| NC_002017.1 | Influenza A virus (A/Puerto Rico/8/1934(H1N1)) | 1778 | 13150 | 13.52% |
| | segment 4, complete sequ | | | |
| NC_003467.2 | Andes virus complete genome | 3671 | 12100 | 30.34% |
| NC_003696.1 | Eyach virus complete genome | 4349 | 29220 | 14.88% |
| NC_004109.1 | La Crosse virus complete genome | 4407 | 12490 | 35.28% |
| NC_004180.1 | Colorado tick fever virus complete sequence | 1884 | 29180 | 6.46% |
| NC_004212.1 | Kadipiro virus chromosome complete genome | 3035 | 20990 | 14.46% |
| NC_004217.1 | Banna virus complete sequence | 3048 | 20700 | 14.72% |
| NC_004296.1 | Lassa virus complete sequence | 3402 | 10689 | 31.83% |
| NC_004906.1 | Influenza A virus (A/Hong Kong/1073/99(H9N2)) | 890 | 13150 | 6.77% |
| | segment 8, complete | | | |
| NC_004908.1 | Influenza A virus ha gene for Hemagglutinin, | 1714 | 13150 | 13.03% |
| | genomic RNA, strain A/Hong | | | |
| NC_004909.1 | Influenza A virus na gene for neuraminidase, genomic | 1418 | 13150 | 10.78% |
| | RNA, strain A/Hong | | | |
| NC_005215.1 | Sin Nombre virus complete sequence | 3696 | 12317 | 30.01% |
| NC_005220.1 | Uukuniemi virus chromosome complete genome | 3120 | 11372 | 27.44% |
| NC_005223.1 | Puumala virus complete sequence | 3480 | 12062 | 28.85% |
| NC_005228.1 | Tula virus complete sequence | 3694 | 12066 | 30.61% |
| NC_005300.2 | Crimean-Congo hemorrhagic fever virus complete | 5366 | 19142 | 28.03% |
| | sequence | | | |
| NC_005775.1 | Oropouche virus complete genome | 4320 | 11985 | 36.05% |
| NC_005894.1 | Pirital virus complete sequence | 3393 | 10482 | 32.37% |
| NC_006317.1 | Sabia virus complete genome | 3366 | 10499 | 32.06% |
| NC_006437.1 | Hantavirus Z0 chromosome complete genome | 3616 | 11850 | 30.51% |
| NC_006506.1 | Thogoto virus | 1560 | 10461 | 14.91% |
| NC_007026.1 | Human picobimavirus RNA complete sequence | 2525 | 4270 | 59.13% |
| NC_007357.1 | Influenza A virus (A/Goose/Guangdong/1/96(H5N1)) | 2341 | 13150 | 17.80% |
| | polymerase (PB2) ge | | | |
| NC_007358.1 | Influenza A virus (A/Goose/Guangdong/1/96(H5N1)) | 2341 | 13150 | 17.80% |
| | polymerase (PB2) | | | |
| NC_007359.1 | Influenza A virus | 2233 | 13150 | 16.98% |
| | (A/goose/Guangdong/1/1996(H5N1)) polymerase | | | |
| | (PA) an | | | |
| NC_007361.1 | Influenza A virus (A/Goose/Guangdong/1/96(H5N1)) | 1458 | 13150 | 11.09% |
| | neuraminidase (NA) | | | |
| NC_007362.1 | Influenza A virus | 1760 | 13150 | 13.38% |
| | (A/goose/Guangdong/1/1996(H5N1)) hemagglutinin | | | |
| | (HA | | | |
| NC_007363.1 | Influenza A virus | 1027 | 13150 | 7.81% |
| | (A/goose/Guangdong/1/1996(H5N1)) segment 7, | | | |
| | comple | | | |
| NC_007364.1 | Influenza A virus | 865 | 13150 | 6.58% |
| | (A/goose/Guangdong/1/1996(H5N1)) segment 8, | | | |
| | comple | | | |
| NC_007366.1 | Influenza A virus (A/New York/392/2004(H3N2)) | 1762 | 13150 | 13.40% |
| | segment 4, complete seq | | | |
| NC_007374.1 | Influenza A virus (A/Korea/426/1968(H2N2)) | 1773 | 13150 | 13.48% |
| | segment 4, complete | | | |
| NC_007543.1 | Rotavirus C | 1110 | 17889 | 6.20% |
| NC_007553.1 | Adult Diarrheal rotavirus J19 | 1287 | 17961 | 7.17% |
| NC_007737.1 | Liao ning virus complete genome | 3055 | 20739 | 14.73% |
| NC_007903.1 | Mobala virus complete sequence | 3380 | 10705 | 31.57% |
| NC_007905.1 | Ippy virus complete sequence | 3366 | 10682 | 31.51% |
| NC_009895.1 | Akabane virus complete sequence | 4309 | 12035 | 35.80% |
| NC_010253.1 | Allpahuayo virus complete sequence | 3343 | 10396 | 32.16% |
| NC_010254.1 | Cupixi virus complete sequence | 3332 | 10438 | 31.92% |
| NC_010256.1 | Bear Canyon virus complete sequence | 3339 | 10441 | 31.98% |
| NC_010562.1 | Chapare virus complete sequence | 3357 | 10464 | 32.08% |
| NC_010700.1 | Whitewater Arroyo virus complete sequence | 3316 | 10448 | 31.74% |
| NC_010701.1 | Tamiami virus (small) complete genome | 3533 | 10647 | 33.18% |
| NC_010708.1 | Thottapalayam virus complete sequence | 3480 | 11732 | 29.66% |
| NC_011509.2 | Rotavirus A | 1356 | 18034 | 7.52% |
| NC_014396.1 | Rift Valley fever virus complete genome | 3885 | 11979 | 32.43% |
| NC_014527.1 | Great Island virus complete genome | 1666 | 17858 | 9.33% |
| NC_015373.1 | Candiru virus complete genome | 4206 | 12495 | 33.66% |
| NC_015411.1 | Sandfly Sicilian Turkey virus complete genome | 4283 | 12603 | 33.98% |
| NC_015450.1 | Aguacate virus complete genome | 4185 | 12408 | 33.73% |
| NC_016152.1 | Luna virus complete genome | 3377 | 10637 | 31.75% |
| NC_018138.1 | SFTS virus HB9 complete genome | 3378 | 11490 | 29.40% |
| NC_018459.1 | Aino virus Gn-Gc-NSm gene forpolyprotein genomic | 4335 | 12135 | 35.72% |
| | RNA isolate 8K | | | |
| NC_018466.1 | Sathuperi virus Gn-Gc-NSm gene forpolyprotein | 4200 | 12034 | 34.90% |
| | genomic RNA | | | |
| NC_018467.1 | Shamonda virus Gn-Gc-NSm gene forpolyprotein | 4314 | 12104 | 35.64% |
| | genomic RNA isolate Ib A | | | |
| NC_018478.1 | Simbu virus Gn-Gc-NSm gene forpolyprotein | 4417 | 12172 | 36.29% |
| | genomic RNA isolate SA Ar | | | |
| NC_018710.1 | Lunk virus NKS- complete genome | 3419 | 10614 | 32.21% |
| NC_021544.1 | Human Rotavirus B Bang373 | 1269 | 17934 | 7.08% |
| NC_021588.1 | Rotavirus G (chicken) | 1267 | 18186 | 6.97% |
| NC_021635.1 | Rotavirus F (chicken) | 1314 | 18341 | 7.16% |
| NC_022037.1 | Brazoran virus complete sequence | 1560 | 13242 | 11.78% |
| NC_023633.1 | Arumowot virus complete sequence | 3848 | 12262 | 31.38% |
| U40822.1 | HPV type 74L1/E6/E7 | 2160 | 7887 | 27.39% |
| X67160.1 | HPV type68 E6/E7 | 1313 | 7838 | 16.75% |
| X67161.1 | HPV type68 L1 | 1680 | 7838 | 21.43% |
| NC_045512.2 | SARS-CoV-2 | 29903 | 29903 | 100.00% |

We claim:

1. A method for detecting a plurality of RNA and DNA viruses in a human biological sample comprising RNA and DNA, the method comprising:
(i) performing reverse transcription of the RNA in the sample using a plurality of DNA primers to prepare double-stranded cDNA of the RNA;
(ii) fragmenting the cDNA and DNA in the sample to prepare DNA fragments;
(iii) treating the DNA fragments with enzymes that repair overhangs to obtain blunt-ended DNA fragments;
(iv) treating the blunt-ended DNA fragments with an enzyme that adds a 3′ adenine overhang to the blunt-ended DNA fragments to obtain 3′-adenine extended DNA fragments;
(v) ligating an adapter comprising an index sequence and a primer target sequence to the 3′-adenine extended DNA fragments to obtain adapter-ligated DNA fragments;
(vi) amplifying the adapter-ligated DNA fragments with a plurality of DNA primer pairs that hybridize to the primer target sequence to obtain an amplified DNA sample;
(vii) contacting the amplified DNA sample with a plurality of tagged RNA probes that hybridize to the amplified DNA sample to provide tagged RNA:DNA hybrid molecules;
(viii) capturing the tagged RNA:DNA hybrid molecules using a molecule that binds to the tag of the tagged RNA:DNA hybrid molecules;
(ix) amplifying the captured, tagged RNA:DNA hybrid molecules using a plurality of DNA primer pairs to obtain a further amplified DNA sample; and
(x) analyzing the further amplified DNA sample based on the index sequence to detect the plurality of RNA and DNA viruses in the human biological sample.

2. The method of claim 1, wherein the DNA is fragmented by sonication.

3. The method of claim 1, wherein the fragmented DNA is on average between 50 and 300 base pairs in length.

4. The method of claim 1, wherein the enzymes of step (iii) have 5′-3′ polymerase activity and 3′-5′ exonuclease activity.

5. The method of claim 1, wherein the enzyme of step (iv) is a polymerase.

6. The method of claim 5, wherein the enzyme is Taq polymerase.

7. The method of claim 1, wherein the adapter that is ligated comprises an index sequence that is 5 to 15 base pairs in length.

8. The method of claim 1, wherein the number of cycles of amplification of step (vi) is tuned based on the concentration of the adapter ligated fragments, such that the adapter ligated fragments are amplified to an appropriate concentration.

9. The method of claim 1, wherein the amplified DNA sample is concentrated to at least about 215 ng/μl.

10. The method of claim 1, wherein the tagged RNA probes of step (vii) are designed to bind to the genomic segment of multi-partite viral genomes which encode the viral capsid.

11. The method of claim 1, wherein the tagged RNA probes of step (vii) are designed to bind to the viral genome of SARS-CoV-2.

12. The method of claim 1, wherein the tagged RNA probes of step (vii) are designed to bind to the L1 gene sequence for every known human papilloma virus.

13. The method of claim 1, wherein the tagged RNA probes of step (vii) are tagged with biotin.

14. The method of claim 1, wherein the tagged RNA probes of step (vii) are tagged with digoxigenin (DIG).

15. The method of claim 1, wherein the hybridization of step (vii) occurs at a temperature of 60-70 degrees Celsius.

16. The method of claim 1, wherein the hybridization of step (vii) is incubated for at least about 18 hours.

17. The method of claim 1, wherein the RNA probes of step (viii) are tagged with biotin and streptavidin binds to the biotin-tagged RNA:DNA hybrid molecules.

18. The method of claim 1, wherein the RNA probes of step (viii) are tagged with digoxigenin and anti-digoxigenin binds to the digoxigenin-tagged RNA:DNA hybrid molecules.

19. The method of claim 1, wherein the molecule that binds to the tag of the tagged RNA:DNA hybrid molecules is linked to a bead.

20. The method of claim 19, wherein the beads are magnetic.

21. The method of claim 1, wherein step (x) comprises next-generation DNA sequencing.

22. The method of claim 21, wherein the DNA sequencing comprises paired-end sequencing.
